(19) United States
(12) Patent Application Publication
(10) Pub. No.: US 2005/0250836 A1
(43) Pub. Date: Nov. 10, 2005

(54) Inhibitors of checkpoint kinases (Wee1 and Chk1)

(75) Inventors: Richard John Booth, Ann Arbor, MI (US); Ho Huat Lee, Epsom (NZ); Alan Kraker, Ann Arbor, MI (US); Daniel Fred Ortwine, Saline, MI (US); Brian Desmond Palmer, Glendene (NZ); Derek J. Sheehan, Dexter, MI (US); Peter Laurence Toogood, Ann Arbor, MI (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/120,746

(22) Filed: May 2, 2005

Related U.S. Application Data
(60) Provisional application No. 60/567,726, filed on May 3, 2004.

(51) Int. Cl. A61K 31/407; C07D 487/02

(52) U.S. Cl. 514/411; 548/429

(57) ABSTRACT

This invention relates to substituted indacene molecules that specifically inhibit one or both of the checkpoint kinases Wee1 and Chk1 and are useful in the treatment of proliferative disorders.

This application claims the benefit of U.S. Provisional Application No. 60/567,726 filed May 3, 2004, the entire disclosure of which is incorporated herein be reference.

### FIELD OF THE INVENTION

This invention relates to small chemical molecules that specifically inhibit one or both of the checkpoint kinases Wee1 and Chk1 and are useful in the treatment or inhibition of cell proliferative diseases such as angiogenesis, atherosclerosis, restenosis, and cancer.

### BACKGROUND OF THE INVENTION

Small molecule inhibitors of Wee1 kinase have been reported (see WO 0119825 and Cancer Res. (2001), 61(22), 8211-8217). Small molecule inhibitors of Chk1 kinase have also been reported WO0016781, Cancer Res. (2000), 60(3), 566-572.

Pyrrolocarbazole derivatives are known to have inhibitory activity against Protein kinase C and anti tumor activity (U.S. Pat. No. 4,912,107) but compared to the compounds of the present invention, the compounds disclosed in U.S. Pat. No. 4,912,107 have very low checkpoint kinase abrogator activity. Pyrrolocarbazole derivatives are also known to stimulate platelet production (WO96/28447) and to promote thrombopoiesis (WO9809967). EP 0695755 discloses another pyrrolocarbazole derivative having Protein kinase c activity. U.S. Pat. No. 5,166,204 discloses antitumor isoindole derivatives having a linkage or lower alkylene group bonded to the 2 and 3 or 3 and 4 of a carbazole skeleton. U.S. Pat. No. 5,728,709 discloses pyrrolocarbazole derivatives that stimulate platelet production. WO 01/85686 also discloses pyrrolocarbazole derivatives.

### SUMMARY OF THE INVENTION

This invention comprises compounds of Formula I: 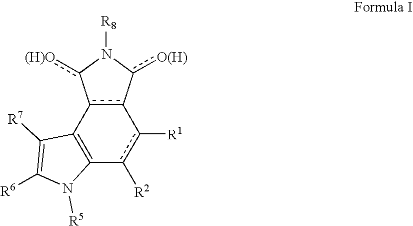
wherein:
each dashed line represents an optional bond;
R¹ is selected from hydrogen, halogen, alkyl; or
a) a phenyl or heteroaryl ring optionally substituted with up to five substituents selected from halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₘR⁴, NHCONR³R⁴, NR³CONHR⁴; or
b) a cycloalkyl or cycloalkenyl ring optionally substituted with up to five substituents selected from, halogen, alkyl, hydroxyl, nitro, cyano, C(O)R³, OR³, S(O)ₘR³, NR³R⁴, OC(O)R³, NR³(CO)OR⁴, CH₂NR³R⁴, CH₂OR³ COOR³, CONR³R⁴, NR³COR⁴, SO₂NR³R⁴, CONHSO₂R³, NR₃S(O)ₘR⁴, NHCONR³R⁴, NR³CONHR⁴; or
c) a heterocyclic ring optionally substituted with up to five substituents selected from, halogen, alkyl, hydroxyl, nitro, cyano, C(O)R³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR₃S(O)ₘR⁴ NHCONR³R⁴, NR³CONHR⁴;
R² is selected from hydrogen, halogen, alkyl; or a cycloalkyl or cycloalkenyl ring optionally substituted with up to five substituents selected from, halogen, alkyl, hydroxyl, nitro, cyano, C(O)R³, OR³, S(O)ₘR³, NR³R⁴, OC(O)R³, NR³(CO)OR⁴, CH₂NR³R⁴, CH₂OR³ COOR³, CONR³R⁴, NR³COR⁴, SO₂NR³R⁴, CONHSO₂R³, NR₃S(O)ₚR⁴, NHCONR³R⁴, NR³CONHR⁴; or
R³ and R⁴ are independently selected from hydrogen, alkyl, or a substituted or unsubstituted carbocyclic group selected from cycloalkyl, cycloalkenyl, heterocyclyl, aryl, and heteroaryl, wherein the said alkyl, or a substituted or unsubstituted carbocyclic group may be optionally substituted with up to 4 groups selected from halogen, hydroxyl, nitro, cyano, alkyl, alkoxy, carboxy, C(O)OH, CONH₂, NHC(O)CH₃, N(CH₃)₂, NHCH₃ thiomethyl, thioethyl, SOCH₃, SO₂CH₃, NHC(O)NR³R⁴, NR³C(O)NHR⁴; or
R³ and R⁴ together with the carbon atom or heteroatom to which they are attached may form a cycloalkyl or heterocyclyl group optionally substituted with up to four groups independently selected from halogen, hydroxyl, nitro, cyano, alkyl, alkyloxy, formyl, carboxy, acetyl, CH₂NH₂, CH₂OH, COOH, CONH₂, NHCOCH₃, N(CH₃)₂, thiomethyl, thioethyl, SOCH₃, SO₂CH₃, alkoxycarbonyl, alkylcarbonyl, alknylamino, aminoalkyl, aminoalkylcarbonyl, amino, mono- or dialkylamino; or
R³ and R⁴ together with the nitrogen to which they are attached may form a heterocyclic ring containing 3-8 members, up to four of which members are optionally carbonyl groups or heteroatoms independently selected from oxygen, sulfur, S(O), S(O)₂, and nitrogen, wherein the carbocyclic group is unsubstituted or substituted with up to four groups independently selected from halogen, hydroxy, hydroxyalkyl, alkyl, alkoxy, alkoxycarbonyl, alkylcarbonyl, alknylamino, aminoalkyl, aminoalkylcarbonyl, amino, mono- or dialkylamino,
R⁵ is selected from hydrogen, alkyl or ((CR⁹R¹⁰)ₙT)ₐ(CR¹¹R¹²)_{b})-Z;
T may be absent, or, when present, is in each instance independently selected from O, CONR³, CONHSO₂, S, S(O)ₘ, NR³, NR³—O, O—S(O)ₘ, S(O)ₘ—O, NR³—S(O)₂, or S(O)₂—NR³;
m=0, 1, 2;
n is in each instance independently 0-6, preferably 0-3;
a is in each instance independently 0-6, preferably 0-3;
b is in each instance independently 0-6, preferably 0-3;
p=1 or 2;
Z is selected from hydrogen, halogen, alkyl, cycloalkyl, cycloalkenyl, heterocyclyl, phenyl, heteroaryl, halogen, cyano, nitro, hydroxy, COR³, CONHSO₂R³, OR³, S(O)ₘR³OSO₂R³, NR³R⁴, CO₂R³, CONR³R⁴, NR³COR⁴, SO₂NR³R⁴, OPO(OR³)(OR⁴), CH═CR³R⁴, CCR³, wherein the alkyl, cycloalkyl, cycloalkenyl, heterocyclyl, phenyl or heteroaryl group may be optionally substituted with up to four groups independently selected from halogen, alkyl, hydroxyl, nitro, cyano, OR³, S(O)ₘR³, NR³R⁴, OC(O)R³, NR³(CO)OR⁴, C(O)R³, COOR³, CONR³R⁴, NR³COR⁴, SO₂NR³R⁴, CONHSO₂R³, NR₃S(O)ₚR⁴, CH₂NR³R⁴, CH₂OR³ NHCONR³R⁴, NR³CONHR⁴;
R⁶ is selected from hydrogen, halogen, CN, alkyl, CH₂OR³, CR⁹═CR¹¹R¹², C≡CR⁹, or ((CR⁹R¹⁰)ₙT)ₐ(CR¹¹R¹²)_{b})-Z; where T, Z, n, a and b are defined as above; or —NC(O)—R³;
a) a phenyl or heteroaryl ring optionally substituted with up to five substituents selected from halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₚR⁴ NHCONR³R⁴, NR³CONHR⁴; or
b) a cycloalkyl or cycloalkenyl ring optionally substituted with up to five substituents selected from, halogen, alkyl, hydroxyl, nitro, cyano, C(O)R³, OR³, S(O)ₘR³, NR³R⁴, OC(O)R³, NR³(CO)OR⁴, CH₂NR³R⁴, CH₂OR³ COOR³, CONR³R⁴, NR³COR⁴, SO₂NR³R⁴, CONHSO₂R³, NR₃S(O)ₚR⁴, NHCONR³R⁴, NR³CONHR⁴; or
c) a heterocyclic ring optionally substituted with up to five substituents selected from, halogen, alkyl, hydroxyl, nitro, cyano, C(O)R³, OR, S(O)ₙR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR₃S(O)ₚR⁴ NHCONR³R⁴, NR³CONHR⁴;
R⁷ is selected from hydrogen, halogen, CN, alkyl, CR⁹═CR¹¹R¹², CCR⁹, or ((CR⁹R¹⁰)ₙT)ₐ(CR¹¹R¹²)_{b})-Z; where T, Z, n, a and b are defined as above;
R⁸ is selected from hydrogen or C₁-C₃ alkyl;
R⁹, R¹⁰, R¹¹ and R¹² are in each instance independently selected from hydrogen, hydroxyl, alkyl, cycloalkyl, cycloalkenyl, phenyl, heteroaryl, heterocyclyl, halogen, cyano, nitro, CH₂NR³R⁴, CH₂OR³, COR³, OR³, S(O)ₘR³, NR³R⁴, COOR³, CONR³R⁴, SO₂NR³R⁴NHCONR³R⁴, NR³CONHR⁴;
wherein the alkyl, cycloalkyl, cycloalkenyl, heterocyclyl, phenyl or heteroaryl group may be optionally substituted with up to four groups independently selected from halogen, alkyl, hydroxyl, nitro, cyano, OR³, S(O)ₙR³, NR³R⁴, OC(O)R³, NR³(CO)OR⁴, C(O)R³, COOR³, CONR³R⁴, NR³COR⁴, SO₂NR³R⁴, CONHSO₂R³, NR₃S(O)ₙR⁴. NHCONR³R⁴, NR³CONHR⁴;
R⁵ and R⁶ or R¹¹ and R¹² together with the carbon atom to which they are attached may form a carbonyl group; or a cycloalkyl or heterocyclyl group, optionally substituted with up to four groups independently selected from halogen, hydroxyl, nitro, cyano, alkyl, halogen, alkyl, nitro, OR³, S(O)ₘR³, NR³R⁴, OC(O)R³, NR³(CO)OR⁴, C(O)R³, COOR³, CONR³R⁴, NR³COR⁴, SO₂NR³R⁴, CONHSO₂R³, NR₃S(O)ₙR⁴, NHCONR³R⁴, NR³CONHR⁴;
or a pharmaceutically acceptable salt, ester or amide form thereof.

It will be understood that the substituent groups depicted by [symbol] O(H) in positions 1 and 3 of Formula I, above, indicate an optional pairing of carbonyl and hydroxyl tautomeric forms.

Among the compounds of this invention are those of Formula I wherein:
R¹ is phenyl optionally substituted by up to three substituents selected from halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₘR⁴, NHCONR³R⁴, NR³CONHR⁴;
R³ and R⁴ are as defined above;
R⁵ is a C₁-C₆ alkyl group optionally substituted by from 1 to 3 groups selected from OH, halogen, —NR³R⁴, —O—(C₁-C₃ alkyl), —O—(C₁-C₃ alkyl)-O—(C₁-C₃ alkyl), with the C₁-C₃ alkyl portions of the last two groups being further optionally substituted by OH, halogen or —NR³R⁴;
R⁶ is H, halogen, C₁-C₆ alkyl, C₁-C₆ alkenyl, —C(O)OH, —C(O)—NR³R⁴, —C(O)—(C₁-C₆ alkyl), —(C₁-C₆ alkyl)-NH—(C₁-C₆ alkyl), —(C₁-C₆ alkyl)-O—(C₁-C₆ alkyl), —(C₁-C₆ alkyl)-O—(C₁-C₆ alkyl)-O—(C₁-C₆ alkyl),
the alkyl portions of each R⁶ group containing an alkyl or alkenyl portion being further optionally substituted by from 1 to 3 groups selected from CN, OH, halogen, —C(O)OH, C(O)O(C₁-C₆ alkyl), which may also include a single —NR³R⁴ or a phenyl, heterocyclyl or heteroaryl group optionally substituted by from 1 to 3 groups selected from halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₚR⁴ NHCONR³R⁴, or NR³CONHR⁴;
R⁷ is H, halogen, C₁-C₆ alkyl, C₁-C₆ alkenyl, C₁-C₆ alkynyl, the alkyl, alkenyl and alkynyl groups being optionally substituted by from 1 to 3 groups selected from oxo, OH, CN, or —NR³R⁴;
R⁸ is selected from hydrogen or C₁-C₃ alkyl;
or a pharmaceutically acceptable salt, ester or amide form thereof.

Another embodiment of this invention comprises compounds of Formula II: 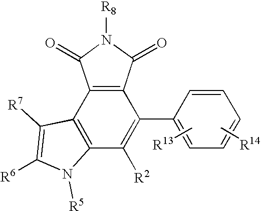
wherein:
R² is H or C₁-C₃ alkyl;
R⁵ is H or C₁-C₆ alkyl optionally substituted by OH, halogen or NR³R⁴;
R¹³ and R¹⁴ are independently selected from H, halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³′ NR³S(O)ₘR⁴, NHCONR³R⁴, NR³CONHR⁴; wherein R³ and R⁴ are as defined above;
R⁶ is H, halogen, C₁-C₆ alkyl, C₁-C₆ alkenyl, —C(O)OH, —C(O)—NR³R⁴, —C(O)—(C₁-C₆ alkyl), —(C₁-C₆ alkyl)-NH—(C₁-C₆ alkyl), —(C₁-C₆ alkyl)-O—(C₁-C₆ alkyl), —(C₁-C₆ alkyl)-O—(C₁-C₆ alkyl)-O—(C₁-C₆ alkyl); the alkyl portions of each R⁶ group containing an alkyl or alkenyl portion being further optionally substituted by from 1 to 3 groups selected from CN, OH, halogen, —C(O)OH, C(O)O(C₁-C₆ alkyl), which may also include a single —NR³R⁴ or a phenyl, heterocyclyl or heteroaryl group optionally substituted by from 1 to 3 groups selected from halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₚR⁴ NHCONR³R⁴, or NR³CONHR⁴;
R⁷ is H, halogen, C₁-C₆ alkyl, C₁-C₆ alkenyl, C₁-C₆ alkynyl, the alkyl, alkenyl and alkynyl groups being optionally substituted by from 1 to 3 groups selected from oxo, OH, CN, or —NR³R⁴; and
R⁸ is H or —CH₃;
or a pharmaceutically acceptable salt, ester or amide form thereof.

Within each of the groups of compounds described above there is a subset of compounds wherein R¹ is a phenyl group ortho-substituted by halogen, —O—C₁-C₃ alkyl, OH, NO₂, NH₂, NH(C₁-C₃ alkyl), N(C₁-C₃ alkyl)₂, or CN. Another subset of compounds within each group herein comprises compounds wherein R⁸ is hydrogen.

### DETAILED DESCRIPTION OF THE INVENTION

The following are definitions of terms used in this specification. The initial definition provided for a group or term herein applies to that group or term throughout the present specification, individually or as part of another group, unless otherwise indicated.

Where stereoisomers or enantiomers exist all possible combinations are claimed.

The terms “halogen” and “halo” refer to fluorine, chlorine, bromine and iodine.

The term “unsaturated ring” includes partially unsaturated and aromatic rings.

The term “alkyl” in the present invention means a straight or branched hydrocarbon radical having from 1 to 8 carbon atoms and includes, for example, methyl, ethyl, n-propyl, isopropyl, n-butyl, sec-butyl, isobutyl, tert-butyl, n-pentyl, iso-pentyl, n-hexyl, and the like. Cycloalkyl and cycloalkenyl refer, respectively, to monocyclic or bridged C₃-C₁₀ cycloalkyl or C₄-C₁₀ cycloalkenyl moieties. Examples of cycloalkyl groups include cyclopropyl, cyclobutyl, cyclopentyl, cycloheptyl, cyclohexyl, and cyclooctyl, cyclononanyl and cyclodecyl rings. Non-limiting examples of cycloalkenyl groups include 1-cyclopenenyl, 2-cyclopentenyl, 3-cyclopentenyl, 1-cyclohexenyl, 2-cyclohexenyl, 3-cyclohexenyl, 1-cycloheptenyl, 2-cycloheptenyl, 3-cycloheptenyl, 2,4-cycloheptadienyl, etc. Bridged moieties include adamantly and bridged, 7-membered norbornyl moieties, such as Bicyclo[2.2.1]heptane.

The term “aryl” means an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl).

The term “heteroaryl” means an aromatic “heterocycle,” “heterocyclic,” “heterocyclyl,” or “heterocyclo” group as defined below that comprises at least one heteroatom.

The aryl or heteroaryl ring may be optionally substituted with up to five substituents selected from NH(C₁-C₆ alkyl), N(C₁-C₆ alkyl)₂, thio C₁-C₆ alkyl, C₁-C₆ alkoxy, hydroxy, carboxy, C₁-C₆ alkoxycarbonyl, halo, nitrile, and cycloalkyl.

By “alkoxy” is meant straight or branched chain alkoxy groups having 1 to 10 carbon atoms, such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy.

“Alkynyl” means straight and branched hydrocarbon radicals having from 2 to 8 carbon atoms and one triple bond and includes ethynyl, 3-butyn-1-yl, propynyl, 2-butyn-1-yl, 3-pentyn-1-yl, and the like.

The terms “cycloalkyl” and “cycloalkenyl” refer to cyclic hydrocarbon groups of 3 to 8 carbon atoms and includes cyclopropyl, cyclobutyl, cyclopentyl, cyclohexyl, cycloheptyl, cyclooctyl and the like.

“Acyl” means an alkyl, aryl, cycloalkyl, heterocycle, heterocyclic, heterocyclyl, or heterocyclo group bonded through a carbonyl group, i.e., R—C(O)—. Typical acyl groups include acetyl, benzoyl, and the like having from 1-10 carbon atoms, preferably 1-6 carbon atoms.

The term “haloalkyl” means an akyl group substituted with 1 to 6 halogen atoms and include trifluoromethyl, trichloromethyl, tribromomethyl, trifluoroethyl, trifluoropropyl, trifluorobutyl, pentafluoroethyl and the like.

The alkyl, alkenyl, alkoxy, and alkynyl groups described above are optionally substituted, preferably by 1 to 3 groups selected from NH(C₁-C₆ alkyl), N(C₁-C₆ alkyl)₂, phenyl, substituted phenyl, thio C₁-C₆ alkyl, C₁-C₆ alkoxy, hydroxy, carboxy, C₁-C₆ alkoxycarbonyl, halo, nitrile, cycloalkyl, and a 5- or 6-membered carbocyclic ring or heterocyclic ring having 1 or 2 heteroatoms selected from nitrogen, substituted nitrogen, oxygen, and sulfur. “Substituted nitrogen” means nitrogen bearing C₁-C₆ alkyl or (CH₂)ₚPh where p is 1, 2, or 3. Perhalo and polyhalo substitution is also included.

The term “heteroatom” means an oxygen, nitrogen, sulfur, or phosphorous atom.

The terms “heterocycle,” “heterocyclic,” “heterocyclyl,” or “heterocyclo” refer to fully saturated or unsaturated, including aromatic (heteroaryl) or nonaromatic cyclic groups, for example, 4- to 7-membered monocyclic, 7- to 11-membered bicyclic, or 10- to 15-membered tricyclic ring systems, which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system.

Monocyclic heterocyclic groups include, but are not limited to, piperidine, 2,6-dimethylpiperazine, piperazine, n-methylpiperazine, pyrrolidinyl, pyrrolyl, pyrazolyl, oxetanyl, pyrazolinyl, imidazolyl, imidazolinyl, S(O)-imidazoles, S(O)₂-imidazoles oxazolyl, oxazolidinyl, isoxazolinyl, isoxazolyl, thiazolyl, thiadiazolyl, thiazolidinyl, isothiazolyl, isothiazolidinyl, morpholine and dimethylmorpoline, 2-thiophene, thiophene, 1-imidazole, 2-imidazole, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, 2-azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothianyl, tetrazole, SO-triazole, SO₂-triazole and the like.

Bicyclic heterocyclic groups include, but are not limited to, indolyl, benzothiazolyl, benzoxazolyl, benzodioxolyl, benzothienyl, quinuclidinyl, quinolinyl, tetra-hydroisoquinolinyl, isoquinolinyl, benzimidazolyl, benzopyranyl, indolizinyl, benzofaryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl or furo[2,3-b]pyridinyl), dihydroisoindolyl, dihydroquinazolinyl (such as 3,4-dihydro-4-oxo-quinazolinyl), tetrahydroquinolinyl and the like. tricyclic heterocyclic groups include, but are not limited to, carbazolyl, benzindolyl, phenanthrolinyl, acridinyl, phenanthridinyl, xanthenyl and the like.

The term “cancer” includes, but is not limited to, the following cancers: cancers of the breast, ovary, cervix, prostate, testis, esophagus, breast, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia.

The terms “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

The compounds of this invention are capable of further forming pharmaceutically acceptable formulations comprising salts, including but not limited to acid addition and/or base salts, solvates and N-oxides of a compound herein. This invention also provides pharmaceutical formulations comprising a compound of this invention together with a pharmaceutically acceptable carrier, diluent, or excipient therefor. All of these forms are within the present invention.

Pharmaceutically acceptable acid addition salts of the compounds of this invention include salts derived form inorganic acids such as hydrochloric, nitric, phosphoric, sulfuric, hydrobromic, hydriodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are the salts of amino acids such as arginate, gluconate, galacturonate, and the like; see, for example, Berge et al., “Pharmaceutical Salts,” J. of Pharmaceutical Science, 1977; 66:1-19.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metal hydroxides, or of organic amines. Examples of metals used as cations are sodium, potassium, magnesium, calcium, and the like. Examples of suitable amines are N,N□-dibenzylethylenediamine, chloroprocaine, choline, diethanolamine, ethylenediamine, N-methylglucamine, and procaine; see, for example, Berge et al., supra., 1977.

The base addition salts of acidic compounds are prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

Examples of pharmaceutically acceptable, non-toxic esters of the compounds of this invention include C₁-C₆ alkyl esters wherein the alkyl group is a straight or branched chain. Acceptable esters also include C₅-C₇ cycloalkyl esters as well as arylalkyl esters such as, but not limited to benzyl. C₁-C₄ alkyl esters are preferred. Esters of the compounds of the present invention may be prepared according to conventional methods “March's Advanced Organic Chemistry, 5^{th} Edition”. M. B. Smith & J. March, John Wiley & Sons, 2001.

Examples of pharmaceutically acceptable, non-toxic amides of the compounds of this invention include amides derived from ammonia, primary C₁-C₆ alkyl amines and secondary C₁-C₆ dialkyl amines wherein the alkyl groups are straight or branched chain. In the case of secondary amines the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods such as “March's Advanced Organic Chemistry, 5^{th} Edition”. M. B. Smith & J. March, John Wiley & Sons, 2001.

The term “prodrug” refers to compounds that are rapidly transformed in vivo to yield the parent compound of the above formulae, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

The present invention also includes isotopically labelled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

The compounds of Formula I and their pharmaceutically acceptable salts can be administered to mammals via either the oral, parenteral (such as subcutaneous, intraveneous, intramuscular, intrasternal and infusion techniques), rectal, intranasal or topical routes.

This invention also includes a method of treating, modulating or inhibiting the onset or progression of proliferative disorders in a mammal, the method comprising administering to a mammal in need thereof a pharmaceutically effective amount of a compound of this invention, or a pharmaceutically effective salt, ester or amide form thereof. Proliferative disorders which may be treated by the methods herein include angiogenesis, atherosclerosis, restenosis, and cancer.

This invention also comprises a method of inhibiting one or both of the checkpoint kinases Wee1 and Chk1 in a mammal, the method comprising administering to a mammal in need thereof a pharmaceutically effective amount of a compound of this invention, or a pharmaceutically effective salt, ester or amide form thereof.

A pharmaceutically or therapeutically effective amount of a compound described herein will be understood to be at least the minimal amount required to treat, prevent or inhibit the progress of undesirable cell proliferation, such as that seen in diseases such as angiogenesis, atherosclerosis, restenosis, and cancer. A pharmaceutically or therapeutically effective amount of a compound herein may also be characterized as at least the minimum amount necessary to provide local or systemic concentrations sufficient to specifically inhibit one or both of the checkpoint kinases Wee1 and Chk1 sufficiently to prevent, inhibit or limit the further proliferation of cells associated with cell proliferative disorders, such as those described herein.

In general, these compounds are most desirably administered in pharmaceutically effective doses ranging from about 10 to about 10,000 mg per day, in single or divided doses (i.e., from 1 to 4 doses per day), although variations will necessarily occur depending upon the species, weight and condition of the subject being treated and the particular route of administration chosen. However, a pharmaceutically effective dosage level that is in the range of about 0.15 mg to about 150 mg per kg of body weight per day is most desirably employed. Nevertheless, variations may occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects, provided that such higher dose levels are first divided into several small doses for administration throughout the day.

The compounds of the present invention may be administered alone or in combination with pharmaceutically acceptable carriers or diluents by either of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH from about 3 to about 8) if necessary and the liquid diluent first rendered isotonic. These aqueous solutions are suitable for intravenous injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

Additionally, it is also possible to administer the compounds of the present invention topically when treating proliferative cell conditions of the skin and this may be done by way of creams, jellies, gels, pastes, patches, ointments and the like, in accordance with standard pharmaceutical practice.

The activity of the compounds of the present invention is determined by their ability to act as check point abrogators. Checkpoint abrogators inhibit kinases involved in the regulation of the G₂/M checkpoint resulting in the reversal of the imposed checkpoint. Wee1 or Chk1 are examples of such kinases. Utilization in cells either having damaged DNA, for instance, but not limited to, DNA damaged by conventional DNA-directed chemotherapeutic agents or by radiation of cells with undamaged DNA presents an opportunity to utilize cellular regulatory pathways to inappropriately and prematurely cause cells to progress into M phase. Such cells may be less likely to survive and further divide since the commitment to M phase was made in the presence of potentially catastrophically damaged DNA. In the case of undamaged cells having no detectable DNA damage, treatment of these cells with the checkpoint abrogators of the present invention may be forced into M phase prematurely with similar cytotoxic effects. (Alan J. Kraker and Robert N. Booher, “New Cell Cycle Targets,”, Ann. Rep. Med. Chem., 1999; 34:247-256).

One can identify the checkpoint abrogators of the present invention by measuring the activity in the assays described in Examples 483 (Wee1) and 484 (Chk1) and 486 (PKC) and selecting those compounds that have at least 10-fold less activity in the PKC assay than they have in the Wee1 assay or at least 5-fold less activity in the PKC assay than they have in the Chk1 assay.

The present invention is illustrated by the following examples. It will be understood, however, that the invention is not limited to the specific details of these examples. Melting points are uncorrected. Proton nuclear magnetic resonance spectra (¹H NMR) and ¹³C nuclear magnetic resonance spectra were measured for solutions in deuterochloroform (CDCl₃) or in CD₃OD or CD₃SOCD₃ and peak positions are expressed in parts per million (ppm) downfield from tetramethylsilane (TMS). The peak shapes are denoted as follows: s, singlet; d, doublet; t, triplet; q, quartet; m, multiplet; b, broad.

The compounds of the present invention can exist in unsolvated forms as well as solvated forms, including hydrated forms. In general, the solvated forms, including hydrated forms, are equivalent to unsolvated forms and are intended to be encompassed within the scope of the present invention.

The term “animal” refers to mammals, including rodents, bovine, equine, canine, feline, and human.

The compounds of the present invention are useful for treating or inhibiting the growth or advancement of cancer (for example, leukemia and cancer of the lung, breast, prostate, and skin such as melanoma) and other proliferative diseases including but not limited to psoriasis, HSV, HIV, restenosis, and atherosclerosis in combination with other conventional therapies. To utilize a compound of the present invention to treat or inhibit cancer, a patient in need of such treatment, such as one having cancer or another cell proliferative diseases.

Furthermore, the compounds of the present invention are useful for treating or inhibiting cancers when combined as adjuvant therapy with other clinical treatment agents and modalities such as, but not limited to, X-irradiation, beam therapy, conventional chemotherapeutic agents such as gemcitabine, paclitaxel, docetaxel, cisplatin, carboplatin, etoposide, adriamycin, topotecan, CPT-11, capecitabine, or ionizing radiation alkylating agents, antimetabolites, antibodies, DNA intercalators, or other such anti-proliferative agents ultimately leading to DNA damage. As new antineoplastic agents or modalities are discovered, the use of the Wee1 and/or Chk1 inhibitors in combination with these other therapeutic agents or modalities is contemplated.

While the in vivo tests described herein teach administering the compounds according to Formula I simultaneously with or subsequent to the administration of the conventional agent, it is contemplated that the compound of the present invention may also be administered prior to the conventional chemotherapeutic agent or agents.

The following examples illustrate particular embodiments of the invention and are not intended to limit the specification, including the claims, in any way. Those skilled in the art will appreciate that numerous changes and modifications can be made to the preferred embodiments of the invention and that such changes and modifications can be made without departing from the spirit of the invention. It is, therefore, intended that the cover all such equivalent variations as fall within the true spirit and scope of the invention.

### Scheme 1: Assembly of the Core Molecule

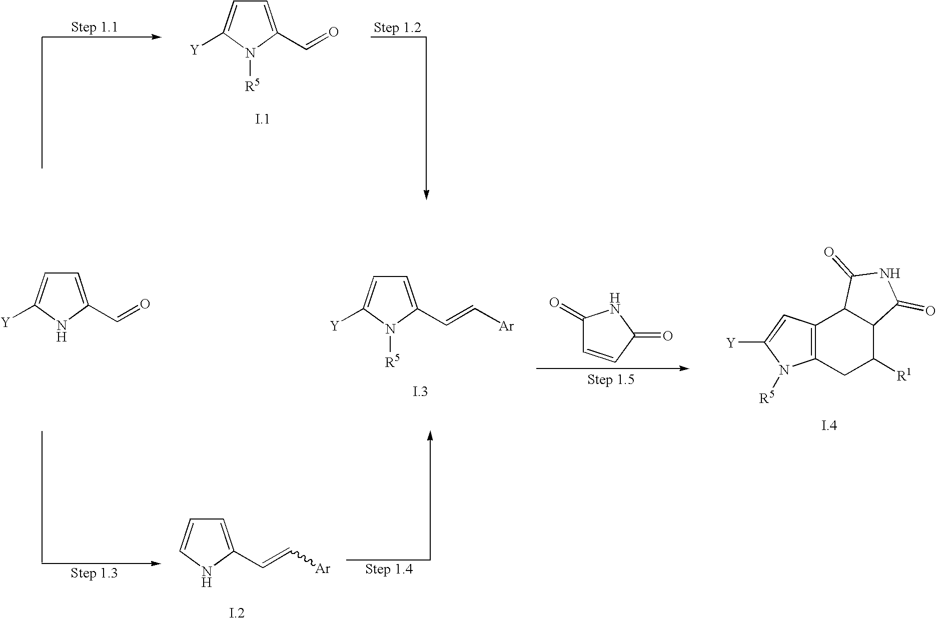

### Procedures for Scheme 1

### EXAMPLE 1

### (2-Chlorobenzyl)triphenylphosphonium Chloride

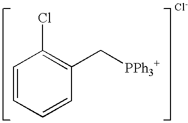

A solution of triphenylphosphine (322 g, 2 mol) and 2-chlorobenzyl chloride (525 g, 2 mol) in xylenes (3000 mL) was refluxed overnight, and then cooled to room temperature. The white solid was collected by filtration and dried in vacuum oven giving 665 g (78.5%) of the title compound as a white solid; LCMS (20 mm C18-MAX-RP column, gradient method 2-95% acetonitrile/water with a 0.1% formic acid buffer in 3.3 min with 2 min hold at 95% acetonitrile in water with (0.1% formic acid), MSD in ESI positive mode) 79.0% at 2.73 min (UV_{210 nm}), API-ES 387.0 (100), 388.2 (25), 389.1 (30)

### EXAMPLE 2

### 2-[2-(2-Chlorophenyl)vinyl]-1-methyl-1H-pyrrole

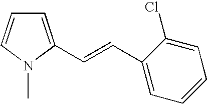

To a suspension of (2-chlorobenzyl)triphenylphosphonium chloride (485 g, 1.15 mol), prepared as described in Example 1, and 1-methyl-2-pyrrolecarboxaldehyde (107 g, 0.98 mol) in dry THF was slowly added sodium ethoxide (21 wt % solution in denatured ethyl alcohol, 520 mL, 1.39 mol). The reaction mixture was stirred at 55° C. overnight under a nitrogen atmosphere. The reaction mixture was cooled to room temperature and the solid material filtered and washed with heptane. The filtrate was concentrated and the residue triturated with heptane and filtered. The clear filtrate was passed through a plug of silica gel and concentrated to give 227 g of title compound as a mixture of geometrical isomers which was used without further purification; LCMS (20 mm C18-MAX-RP column, gradient method of 2-95% acetonitrile in water with 0.1% formic acid in 3.3 min with 2 min hold at 95% acetonitrile/water (0.1% formic acid), MSD in ESI positive mode) 48.8% at 2.71 min, 32.1% at 3.92 min (UV_{210 nm}); API-ES 218.1 (100), 219.0 (15), 219.9 (30), 304.8 (10).

### EXAMPLE 3

### 4-(2-Chlorophenyl)-6-methyl-4,5,6,8b-tetrahydro-3aH-2,6-diaza-as-indacene-1,3-dione

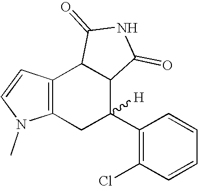

A solution of 2-[2-(2-chlorophenyl)vinyl]-1-methyl-1H-pyrrole (217.5 g, 1 mol), prepared as described in Example 2 and maleimide (135.8 g, 1.4 mol) in tetrahydronaphthalene (2500 mL) was refluxed overnight. The reaction mixture was cooled to 100° C. and heptane (4000 mL) was added. The solution was stirred and further cooled to room temperature. The yellow solid was filtered, washed with heptane and dried in high vacuum at 40° C. over giving 370 g of the title compound (mixture of isomers) as a yellow solid which was used without further purification; LCMS (20 mm C18-MAX-RP column, gradient method 2-95% acetonitrile in water and 0.1% formic acid) in 3.3 min with 2 min hold at 95% acetonitrile/water (0.1% formic acid), MSD in ESI positive mode) 15.1% at 3.18 min, 34.5% at 3.25 min (UV_{210 nm}); API-ES 315.0 (100), 315.9 (15), 317.1 (30), 336.9 (20), 338.0 (5)

### EXAMPLE 4

### 2-[2-(2-Methoxyphenyl)vinyl]-1-methyl-1H-pyrrole

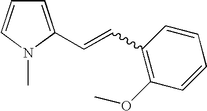

Lithium diisopropylamide (16.49 mL of a 2N solution, 0.033 mol) was added dropwise under nitrogen to a suspension of (2-methoxybenzyl)triphenylphosphonium bromide (14.01 g, 0.030 mol) in dry tetrahydrofuran (250 mL). After stirring at room temperature for 15 min a solution of 1-methylpyrrole-2-carboxaldehyde (3.00 g, 0.027 mol) in tetrahydrofuran (25 mL) was added and the solution was stirred for a further 16 h. Water was added and the mixture was extracted with ethyl acetate and the organic portion was worked up to give an oil which was chromatographed on silica. Elution with ethyl acetate/petroleum ether (1:4) gave 2-[2-(2-methoxyphenyl)vinyl]-1-methyl-1H-pyrrole as a mixture of E/Z isomers, as a yellow oil (4.69 g, 86%). ¹H NMR (400 MHz, CDCl₃) δ ppm 3.53 (s), 3.69 (s), 3.83 (s), 3.88 (s), 5.97-6.03 (m), 6.14-6.16 (m), 6.44 (d, J=12.2 Hz), 6.49-6.56 (m), 6.61-6.63 (m), 6.82-6.97 (m), 7.00 (d, J=16.3 Hz), 7.17-7.25 (m), 7.38 (dd, J=7.6, 1.5 Hz), 7.52 (dd, J=7.6, 1.6 Hz). API-CI Found: 214 (M+1).

### EXAMPLE 5

### 4-(2-Methoxyphenyl)-6-methyl-4,5,6,8b-tetrahydro-3aH-2,6-diaza-as-indacene-1,3-dione

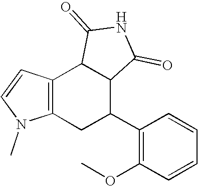

A mixture of the 2-[2-(2-methoxyphenyl)vinyl]-1-methyl-1H-pyrrole (7.00 g, 0.033 mol), prepared as described in Example 4 and maleimide (4.77 g, 0.049 mol) was warmed neat at 185° C. for 3.5 h. Diethyl ether was added to the cooled melt and the mixture was stirred overnight. The resulting tan precipitate was filtered off and washed well with ether, leaving 4-(2-methoxyphenyl)-6-methyl-4,5,6,8b-tetrahydro-3aH-2,6-diaza-as-indacene-1,3-dione as a tan powder (7.63 g, 75%), mp 208-212° C. API-CI Found: 311 (M+1). This material was used directly.

### EXAMPLE 6

### 2-[2-(2-Chloro-6-methoxyphenyl)vinyl]-1-methyl-1H-pyrrole

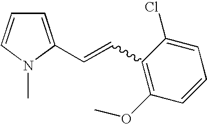

Reaction of 1-methylpyrrole-2-carboxaldehyde with (2-chloro-6-methoxybenzyl)triphenylphosphonium chloride as described for Example 4 gave 2-[2-(2-chloro-6-methoxyphenyl)vinyl]-1-methyl-1H-pyrrole (93%) as a yellow oil, as a mixture of E/Z isomers, which was used directly. ¹H NMR (400 MHz, DMSO-D6) δ ppm 3.64 (s,), 3.80 (s), 3.88 (s), 3.94 (s), 6.03-6.06 (m), 6.43-6.46 (m), 6.79-6.81 (m), 6.93-7.07 (m), 7.15-7.20 (m), 7.42 (d, J=16.3 Hz). API-CI Found: 248, 250 (M+1).

### EXAMPLE 7

### 4-(2-Chloro-6-methoxyphenyl)-6-methyl-4,5,6,8b-tetrahydro-3aH-2,6-diaza-as-indacene-1,3-dione

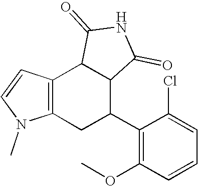

Reaction of the diene 2-[2-(2-chloro-6-methoxyphenyl)vinyl]-1-methyl-1H-pyrrole prepared as described in Example 6 with maleimide at 185° C. as described in EXAMPLE 5 gave 4-(2-chloro-6-methoxyphenyl)-6-methyl-4,5,6,8b-tetrahydro-3aH-2,6-diaza-as-indacene-1,3-dione as a tan powder (78%), which was used directly, mp 186-188° C. API-CI Found: 345, 347 (M+1).

### EXAMPLE 8

### 2-[2-(2-Chlorophenyl)vinyl]-1-[2-(2-ethoxyethoxy)ethyl]-1H-pyrrole

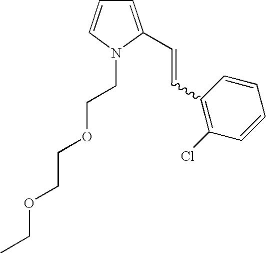

1-[2-(2-Ethoxyethoxy)ethyl]-1H-pyrrole-2-carbaldehyde prepared as described in Example 18 was reacted with (2-chlorobenzyl)triphenylphosphonium chloride and LDA as described in Example 4 to give 2-[2-(2-chlorophenyl)vinyl]-1-[2-(2-ethoxyethoxy)ethyl]-1H-pyrrole as a yellow oil, as a mixture of E/Z isomers (91%). ¹H NMR (400 MHz, DMSO-D6) δ ppm 1.03 (t, J=7.01 Hz), 1.08 (t, J=7.1 Hz), 3.35 (q, J=7.1 Hz), 3.39-3.43 (m), 3.44-3.51 (m), 3.62-3.66 (m), 4.09 (t, J=5.7 Hz), 4.22 (t, J=5.5 Hz), 5.60 (dd, J=3.7, 1.4 Hz), 5.83 (t, J=3.5 Hz), 6.07 (t, J=3.1 Hz), 6.35 (d, J=12.1 Hz), 6.51 (dd, J=3.7, 1.5 Hz), 6.67 (d, J=12.1 Hz), 6.77 (t, J=2.4 Hz), 6.88 (dd, J=2.4, 1.8 Hz), 7.07 (d, J=16.0 Hz), 7.20-7.36 (m), 7.43 (dd, J=7.9, 1.2 Hz), 7.86 (dd, J=7.9, 1.5 Hz). API-CI Found: 318, 320 (M−1).

### EXAMPLE 9

### 4-(2-Chlorophenyl)-6-[2-(2-ethoxyethoxy)ethyl]-4,5,6,8b-tetrahydro-3aH-2,6-diaza-as-indacene-1,3-dione

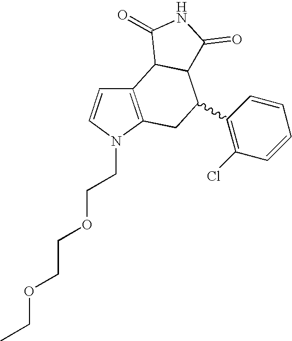

Reaction of 2-[2-(2-chlorophenyl)vinyl]-1-[2-(2-ethoxyethoxy)ethyl]-1H-pyrrole, prepared as described in Example 8 with maleimide as described in Example 5 gave 4-(2-chlorophenyl)-6-[2-(2-ethoxyethoxy)ethyl]-4,5,6,8b-tetrahydro-3aH-2,6-diaza-as-indacene-1,3-dione as a tan solid (74%), which was used directly, mp 158-162° C. API-CI Found: 417, 419 (M+1).

### EXAMPLE 10

### 1-(2-Methoxyethyl)-1H-pyrrole-2-carbaldehyde

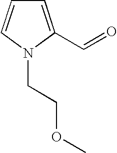

To a suspension of 60% NaH (1.9 g) in anhydrous DMF (50 mL), under an inert atmosphere of argon were added 1H-pyrrole-2-carbaldehyde (5 g) and DMF (10 mL). The reaction mixture was stirred at room temperature for 30 minutes and then 1-bromo-2-methoxy-ethane (6 mL) was added. The reaction mixture was stirred at room temperature overnight. Water (250 mL), ethyl acetate (250 mL) and salt were added, after the layers were separated the product was extracted with ethyl acetate (3×250 mL). The combined ethyl acetate layer was dried (MgSO₄) and concentrated to dryness. Purification by column chromatography on silica using a gradient of ethyl acetate 0% to 6% in DCM and drying under vacuum at 50° C. for 2 hours gave the title compound (3.9 g); ¹H NMR (400 MHz, DMSO-D6) δ ppm 3.19 (s, 3H), 3.55 (t, J=5.37 Hz, 2H), 4.42 (t, J=5.37 Hz, 2H), 6.21 (dd, J=4.03, 2.56 Hz, 1H), 7.02 (dd, J=3.91, 1.71 Hz, 1H), 7.26 (s, 1H), 9.49 (d, J=0.98 Hz, 1H). Mass Spectrum APCI+ 154.

### EXAMPLE 11

### 2-[2-(2-Chlorophenyl)vinyl]-1-(2-methoxyethyl)-1H-pyrrole

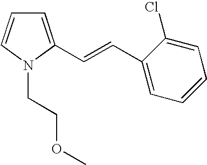

To a suspension of (2-chlorobenzyl)triphenylphosphonium chloride (8.304 g) in anhydrous THF (50 mL), under an inert atmosphere of argon, was added LDA (2.0 M solution in heptane/tetrahydrofuran/ethylbenzene) (9.8 mL). The reaction mixture was stirred at room temperature for 30 minutes, then 1-(2-Methoxyethyl)-1H-pyrrole-2-carbaldehyde (2.0 g), prepared as described in Example 10 was added in THF (3 mL). The reaction mixture stirred at room temperature overnight. In a separate flask, to a suspension of the phosphonium salt (4.15 g) in anhydrous THF (25 mL), under an inert atmosphere of argon, was added LDA (2.0 M solution) (4.9 mL). The reaction mixture was stirred at room temperature for 30 minutes and then added to the reaction mixture above. The reaction mixture was stirred for 46 hours, then a saturated aqueous solution of NH₄Cl (100 mL) was added and the organic components were extracted with ethyl acetate (3×200 mL), dried (MgSO₄) and concentrated to dryness. Purification by column chromatography on silica using dichloromethane as the eluent gave the title compound (1.8 g) as a mixture of isomers. Mass Spectrum ACPI+ MH+ 262, 264.

### EXAMPLE 12

### 4-(2-Chlorophenyl)-6-(2-methoxyethyl)-4,5,6,8b-tetrahydro-3aH-2,6-diaza-as-indacene-1,3-dione

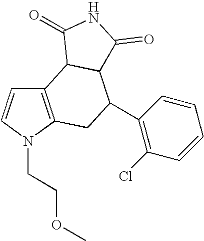

2-[2-(2-Chlorophenyl)vinyl]-1-(2-methoxyethyl)-1H-pyrrole (1.8 g), prepared as described in Example 11 and maleimide (0.8 g) were dissolved in THF (25 mL), the reaction mixture was concentrated to dryness, and then heated at 140° C.-170° C. for one hour. One additional portion of maleimide (0.2 g) was added and the reaction mixture was heated for one additional hour. The reaction mixture was cooled to room temperature. The product was purified by column chromatography using gradient eluent of 10% ethyl acetate to 100% ethyl acetate in hexanes. The title compound, was isolated as a mixture of isomers (1.5 g); NMR (Major isomer reported)¹H NMR (400 MHz, DMSO-D6) δ ppm 2.8 (dd, J=15.0, 4.3 Hz, 1H), 2.95 (d, J=13.7 Hz, 1H), 3.29 (s, 3H), 3.42 (m, 4H), 3.72 (dd, J=7.4, 3.8 Hz, 1H), 3.95 (m, 4H), 5.94 (d, J=2.9 Hz, 1H), 6.62 (t, J=3.1 Hz, 1H), 7.2 (td, J=7.6, 1.5 Hz, 1H), 7.31 (td, J=7.5, 1.3 Hz, 1H), 7.40 (dd, J=7.8, 1.2 Hz, 1H), 7.59 (dd, J=7.9, 1.3 Hz, 1H), 10.71 (s, 1H); Mass Spectrum APCI− (MH−) 359,357.

### EXAMPLE 13

### 1-(3-Methoxypropyl)-1H-pyrrole-2-carbaldehyde

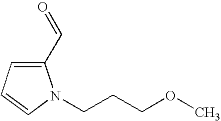

To a stirred mixture of 2-pyrrolecarboxaldehyde (2.12 g, 22.3 mol) and 1-bromo-3-methoxypropane (3.41 g, 22.3 mmol) in DMF (20 mL) at 20° C. (and under a nitrogen atmosphere) was added NaH (1.07 g, 22.3 mmol). The reaction mixture was further stirred at this temperature overnight (16 h). It was partitioned between ethyl acetate and water. The ethyl acetate layer was separated, washed again with water, dried (Na₂SO₄), and evaporated to give the title compound (3.7 g, 99%) as an oil (homogeneous on TLC: chloroform/methanol=50:1); API-CI Found: 168.0 (M+1,100); ¹H NMR (400 MHz, DMSO-D6) δ ppm 1.88 (m, 2H) 3.21 (s, 3H) 3.24 (t, J=6.2 Hz, 2H) 4.30 (t, J=7.1 Hz, 2H) 6.23 (dd, J=4.0 &2.4 Hz, 1H) 7.02 (dd, J=4.0 & 1.8 Hz, 1H) 7.25 (m, 1H) 9.50 (s, 1H); EIMS found M⁺: 167.0945. C₉H₁₃NO₂ requires 167.0946.

### EXAMPLE 14

### 2-[2-(2-Chlorophenyl)vinyl]-1H-pyrrole

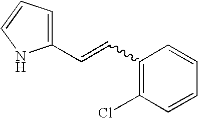

To a stirred heterogeneous mixture of 2-pyrrolecarboxaldehyde (11.6 g, 0.12 mol) and (2-chlorobenzyl)triphenylphosphonium chloride (57.0 g, 0.13 mol) in dichloromethane (500 mL) at 20° C. (under a nitrogen atmosphere) was added drop-wise (over 40 min) 17M NaOH (57.6 mL, 0.98 mol). The reaction mixture was further stirred for an hour. It was washed with water; dried (Na₂SO₄) and evaporated to give an oil. It was purified on a silica column (eluting with dichloromethane) to give the title compound (a mixture of cis- and trans-isomers, ratio ca. 1:1) as an oil (11.1 g, 42%). ¹H NMR (400 MHz, DMSO-D6) δ ppm 5.69 (m, 1H) 5.90 (m, 1H) 6.09 (m, 1H) 6.21 (d, J=2.1 Hz, 1H) 6.30 (m, 1H) 6.53 (d, J=2.1 Hz, 1H) 6.71 (m, 1H) 6.87 (m, 1H) 6.97 (d, J=16.4 Hz, 1H) 7.12 (d, J=16.4 Hz, 1H) 7.21 (m, 1H) 7.31 (m, 3H) 7.43 (dd, J=8.0 & 1.3 Hz, 1H) 7.51 (m, 2H) 7.73 (dd, J=8.0 & 1.5 HZ, 1H) 10.80 (s, exchangeable with D₂O, 1H) 11.32 (s, exchangeable with D₂O, 1H); EIMS found M⁺: 203.0501, 205.0474. C₁₂H₁₀ClN requires 203.0501, 205.0472.

### EXAMPLE 15

### 2-[2-(2-Chlorophenyl)vinyl]-1-(3-methoxypropyl)-1H-pyrrole

Prepared by Method A or Methods B and C 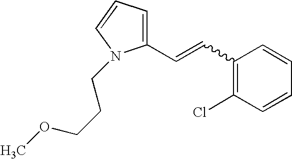
Method A:

To a stirred mixture of 2-[2-(2-chlorophenyl)vinyl]-1H-pyrrole (2.03 g, 10.0 mmol) prepared as described in Example 14 and 1-bromo-3-methoxypropane (1.68 g, 11.0 mmol) in DMF (20 mL) at 20° C. (and under a nitrogen atmosphere) was added NaH (0.29 g, 12.0 mmol). The reaction mixture was further stirred for 1 h. It was quenched with water and ethyl acetate was added. The ethyl acetate layer was separated, further washed with water, dried (Na₂SO₄), and evaporated to give the crude product as a brownish purple oil. It was purified by a silica column (ethyl acetate/petroleum=1:20) to give 2-[2-(2-chlorophenyl)vinyl]-1-(3-methoxypropyl)-1H-pyrrole (a mixture of cis- and trans-isomers, ratio ca. 1.2:1.0) (2.31 g, 84%) as an oil; API-CI Found: 276.0 (M+1, 100%), 278.0 (33); ¹H NMR (400 MHz, DMSO-D6) δ ppm 1.87 (m, 2H) 3.21&3.24 (s&s, 3H) 3.25 (m, 2H) 3.99 & 4.11 (t & t, J=6.9 Hz, 2H) 5.58-6.85 (m, 3H) 6.35 (d, J=12.1 Hz, 0.55H) 6.63 (d, J=12.1 Hz, 0.55H) 7.10 (d, J=16.1 Hz, 0.45H) 7.21 (d, J=16.1 Hz, 0.45H) 7.22-7.88 (m, 4H); EIMS found M⁺: 275.1074, 277.1049. C₁₆H₁₈ClNO requires 275.1076, 277.1047.

Method B:

To a stirred mixture of 1-(3-methoxypropyl)-1H-pyrrole-2-carbaldehyde (3.72 g, 22.3 mmol) prepared as described in Example 13 and (2-chlorobenzyl)triphenylphosphonium chloride (10.4 g, 24.5 mmol) in dichloromethane (100 mL) at 20° C. (under a nitrogen atmosphere) was added drop-wise (over 10 min) 17M NaOH (10.5 mL, 0.18 mol). The reaction mixture was further stirred overnight (20 h). It was worked up and purified as in Method A to give the title compound (a mixture of cis- and trans-isomers, ratio ca. 1:2.8) (0.86 g, 14%).

Method C:

To a stirred heterogeneous mixture of (2-chlorobenzyl)triphenylphosphonium chloride (7.91 g, 18.7 mmol) in THF (30 mL) at 20° C. (under a nitrogen atmosphere) was slowly added lithium diisopropylamide (2.0M solution in THF/n-heptane) (9.35 mL, 18.7 mmol). The reaction mixture was further stirred for an hour. To the resulting homogeneous orange solution was added a solution of 1-(3-methoxypropyl)-1H-pyrrole-2-carbaldehyde (2.84 g, 17.0 mmol) prepared as described in Example 13 in THF (10 mL). The reaction was stirred overnight (19 h). It was partitioned between ethyl acetate and water. The organic layer was separated and washed again with water; dried (Na₂SO₄), evaporated and purified by a silica column (eluting with dichloromethane/petroleum ether=1:1) to give 2-[2-(2-chlorophenyl)vinyl]-1-(3-methoxypropyl)-1H-pyrrole (4.42 g, 98%).

### EXAMPLE 16

### 4-(2-Chlorophenyl)-6-(3-methoxypropyl)-4,5,6,8b-tetrahydro-3aH-2,6-diaza-as-indacene-1,3-dione

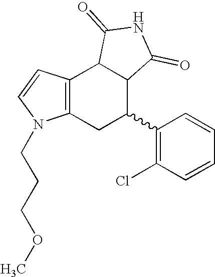

A mixture of 2-[2-(2-chlorophenyl)vinyl]-1-(3-methoxypropyl)-1H-pyrrole (5.27 g, 19.2 mmol) prepared as described in Example 15 and maleimide (2.80 g, 28.9 mmol) was heated at 180-210° C. (bath temperature) for 3 h. It was cooled to room temperature and the crude product dissolved in ethyl acetate. The ethyl acetate solution was washed with water several times, dried (Na₂SO₄) and evaporated to give 4-(2-chlorophenyl)-6-(3-methoxypropyl)-4,5,6,8b-tetrahydro-3aH-2,6-diaza-as-indacene-1,3-dione (a mixture of isomers of ratio ca. 0.5:1.0) (7.13 g, 100%) as a light orange solid; mp 75-90° C.; API-CI Found: 373.0 (M+1, 100), 375.0 (33); 371.0 (M−1,100), 373.0 (33); ¹H NMR (400 MHz, DMSO-D6) δ ppm 1.69 & 1.82 (m & m, ratio ca. 0.5:1.0, 2H) 2.71-4.30 (m, 9H) 3.10 & 3.17 (s & s, ratio ca. 0.5:1.0, 3H) 6.02 & 6.06 (d & d, J=2.8 & 2.8 Hz, ratio ca. 1.0:0.5, 1H) 6.62-6.67 (m, 1H) 7.12-7.76 (m, 4H) 10.75 & 11.02 (br s & br s, ratio ca. 1.0:0.5, 1H); EIMS found: M⁺: 372.1237, 374.1215. C₂₀H₂₁ClN₂O₃ requires 372.1240, 374.1211.

### EXAMPLE 17

### 1-[3-(2-Methoxyethoxy)propyl]-1H-pyrrole-2-carbaldehyde

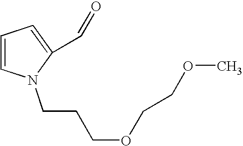

Using the method for preparation of Example 13, 1-[3-(2-methoxyethoxy)propyl]-1H-pyrrole-2-carbaldehyde was prepared from 2-pyrrolecarboxaldehyde (1.90 g, 20.0 mol), 1-bromo-3-(2-methoxyethoxy)propane (3.94 g, 20.0 mmol) and NaH (0.48 g, 20.0 mmol) in DMF (20 mL) at 20° C. (under a nitrogen atmosphere for 24 h). The yield, after chromatography (dichloromethane/ethyl acetate=20:1), was 0.79 g (19%) as a yellow oil; ¹H NMR (400 MHz, DMSO-D6) δ ppm 1.88 (m, 2H) 3.25 (s, 3H) 3.29 (t, J=6.2 Hz, 2H) 3.45 (m, 4H) 4.31 (t, J=6.9 Hz, 2H) 6.23 (dd, J=4.0 &2.4 Hz, 1H) 7.02 (dd, J=4.0 & 1.7 Hz, 1H) 7.24 (br s, 1H) 9.50 (s, 1H); EIMS found M⁺: 211.1206. C₁₁H₁₇NO₃ requires 211.1208.

### EXAMPLE 18

### 1-[2-(2-Ethoxyethoxy)ethyl]-1H-pyrrole-2-carbaldehyde

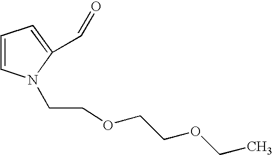

Using the method of Example 13, 1-[2-(2-ethoxyethoxy)ethyl]-1H-pyrrole-2-carbaldehyde was prepared from 2-pyrrolecarboxaldehyde (1.90 g, 20.0 mol), 1-bromo-2-(2-ethoxyethoxy)ethane (3.94 g, 20.0 mmol) and NaH (0.48 g, 20.0 mmol) in DMF (15 mL) at 20° C. (under a nitrogen atmosphere for 17 h). The yield, after chromatography (dichloromethane), was 3.82 g (91%) as an oil; API-CI Found: 212.0 (M+1, 100%); ¹H NMR (400 MHz, DMSO-D6) δ ppm 1.07 (t, J=7.0 Hz, 3H) 3.35-3.48 (m, 6H) 3.65 (t, J=5.5 Hz, 2H) 4.43 (t, J=5.5 Hz, 2H) 6.22 (dd, J=4.0 & 2.5 Hz, 1H) 7.03 (dd, J=4.0 & 1.7 Hz, 1H) 7.28 (br s, 1H) 9.50 (d, J=0.9 Hz, 1H).

### EXAMPLE 19

### 2-[2-(2-Chlorophenyl)vinyl]-1-[3-(2-methoxyethoxy)propyl]-1H-pyrrole

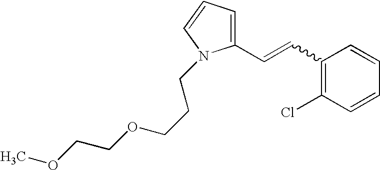

Using method C of Example 15, the title compound was prepared from 1-[3-(2-methoxyethoxy)propyl]-1H-pyrrole-2-carbaldehyde (0.68 g, 3.22 mmol) prepared as described in Example 17, (2-chlorobenzyl)triphenylphosphonium chloride (1.5 g, 3.54 mmol) and lithium diisopropylamide (2.0M solution in THF/n-heptane) (1.77 mL, 3.54 mmol), using a reaction time of 25 h. After purification by a silica column (eluting with dichloromethane/petroleum ether=4:1) 2-[2-(2-chlorophenyl)vinyl]-1-[3-(2-methoxyethoxy)propyl]-1H-pyrrole was obtained as an oil (0.69 g, 67%) (a mixture of cis- and trans-isomers, ratio ca. 1.0:2.2); API-CI Found: 320.0 (M+1, 100%), 322.0 (30); ); ¹H NMR (400 MHz, DMSO-D6) øppm 1.81-1.92 (m, 2H) 3.22&3.27 (s & s, 3H) 3.30 (m, 2H) 3.39-3.51 (m, 4H) 4.00 & 4.12 (t & t, J=6.9 Hz, 2H) 5.58-6.87 (m, 3H) 6.35 (d, J=12.1 Hz, 0.31H) 6.64 (d, J=12.1 Hz, 0.31H) 7.09 (d, J=16.1 Hz, 0.69H) 7.21 (d, J=16.1 Hz, 0.69H) 7.21-7.88 (m, 4H); EIMS found M⁺: 319.1338, 321.1309. C₁₈H₂₂ClNO₂ requires 319.1339, 321.1309.

### EXAMPLE 20

### 4-(2-Chlorophenyl)-6-[3-(2-methoxyethoxy)propyl]-4,5,6,8b-tetrahydro-3 aH-2,6-diaza-as-indacene-1,3-dione

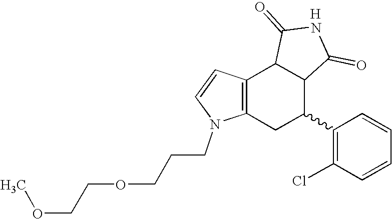

A mixture of 2-[2-(2-chlorophenyl)vinyl]-1-[3-(2-methoxyethoxy)propyl]-1H-pyrrole (0.63 g, 1.98 mmol) prepared as described in Example 19 and maleimide (0.29 g, 2.97 mmol) was heated at 180-210° C. (bath temperature) for 3 h. It was cooled to room temperature and the product dissolved in ethyl acetate. The ethyl acetate solution was washed with water several times, dried (Na₂SO₄), and evaporated to give 4-(2-chlorophenyl)-6-[3-(2-methoxyethoxy)propyl]-4,5,6,8b-tetrahydro-3aH-2,6-diaza-as-indacene-1,3-dione (a mixture of isomers of ratio ca. 1.0:4.0) (0.82 g, 100%) as an orange gum; API-CI Found: 417.0 (M+1, 100), 419.0 (30); 415.0 (M−1, 100), 417.0 (30); ¹H NMR (400 MHz, DMSO-D6) δ ppm 1.70 & 1.82 (2m, ratio ca. 1.0:4.0, 2H) 2.73-4.00 (m, 13H) 3.18 & 3.20 (s & s, ratio ca. 4.0:1.0, 3H) 6.02 & 6.07 (d & d, J=2.8 & 2.8 Hz, ratio ca. 4.0:1.0, 1H) 6.64-6.68 (m, 1H) 7.11-7.67 (m, 4H) 10.81 (br s, 1H); FABMS found [M+H]⁺: 417.1555, 419.1560. C₂₂H₂₆ClN₂O₄ requires 417.1581, 419.1551.

### EXAMPLE 21

### 5-Formyl-1-(3-methoxypropyl)-1H-pyrrole-2-carboxylic acid ethyl ester

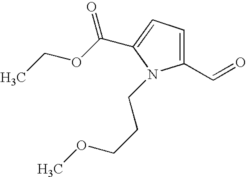

To a stirred solution of 5-formyl-1H-pyrrole-2-carboxylic acid ethyl ester (1.67 g, 10.0 mmol) and 1-bromo-3-methoxypropane (1.53 g, 10.0 mmol) in a mixed solvent of THF (30 mL) and DMF (20 mL) at 20° C. (under a nitrogen atmosphere) was added NaH (0.24 g, 10.0 mmol). After gas evolution had ceased, the reaction mixture was stirred at reflux for 10 h. It was partitioned between ethyl acetate and water and the ethyl acetate layer was separated, washed again with water, dried (Na₂SO₄), and evaporated to give an oil which was applied to a silica column (eluting with ethyl acetate/petroleum ether=1:8) to afford pure 5-formyl-1-(3-methoxypropyl)-1H-pyrrole-2-carboxylic acid ethyl ester (1.33 g, 56%) as an oil; API-CL Found: 240.0 (M+1,100); ¹H NMR (400 MHz, DMSO-D6) δ ppm 1.30 (t, J=7.1 Hz, 3H) 1.89 (m, 2H) 3.19 (s, 3H) 3.28 (t, J=6.2 Hz, 2H) 4.29 (q, J=7.1 Hz, 2H) 4.75 (t, J=7.2 Hz, 2H) 6.93 (d, J=4.2 Hz, 1H) 7.60 (d, J=4.2 Hz, 1H) 9.74 (s, 1H); EIMS found M⁺: 239.1157. C₁₂H₁₇NO₄ requires 239.1157.

### EXAMPLE 22

### 5-[2-(2-Chlorophenyl)vinyl]-1-(3-methoxypropyl)-1H-pyrrole-2-carboxylic acid ethyl ester

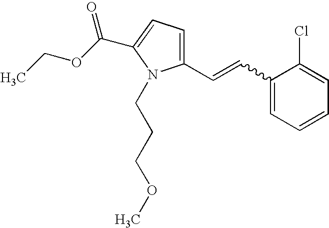

To a stirred heterogeneous mixture of (2-chlorobenzyl)triphenylphosphonium chloride (2.47 g, 5.84 mmol) in THF (10 mL) at 20° C. (under a nitrogen atmosphere) was slowly added lithium diisopropylamide (2.0M solution in THF/n-heptane) (2.92 mL, 5.84 mmol). The reaction mixture was further stirred for 30 min. To the resulting homogeneous orange solution was added a solution of 5-formyl-1-(3-methoxypropyl)-1H-pyrrole-2-carboxylic acid ethyl ester (1.27 g, 5.31 mmol) prepared as described in Example 21 in THF (2.5 mL). The reaction was stirred overnight (16 h). It was partitioned between ethyl acetate and water and the organic layer was separated and washed again with water, dried (Na₂SO₄), evaporated and purified by a silica column (eluting with ethyl acetate/petroleum ether=1:4) to give 5-[2-(2-chlorophenyl)vinyl]-1-(3-methoxypropyl)-1H-pyrrole-2-carboxylic acid ethyl ester (a mixture of cis- and trans-isomers, ratio ca. 1.0:1.8) (1.85 g, 100%) as an orange oil; API-CI Found: 348.0 (M+1, 100%), 350.0 (33); ¹H NMR (400 MHz, DM SO-D6) δ ppm 1.22-1.31 (m, 3H) 1.83-1.93 (m, 2H) 3.17&3.25 (s & s, ratio ca. 1.8:1.0, 3H) 3.25 (t, J=6.0 Hz, 2H) 4.16-4.26 (m, 2H) 4.43 & 4.54 (t & t, J=7.2 & 7.0 Hz, ratio ca. 1.0:1.8, 2H) 5.57 (d, J=4.2 Hz, 0.36H) 6.66 (d, J=4.2 Hz, 0.64H) 6.69 (d, J=4.2 Hz, 0.36H) 6.73 (br s, 0.72H) 6.94 (d, J=4.2 Hz, 0.64H) 7.30 (d, J=16.2 Hz, 0.64H) 7.36 (d, J=16.2 Hz, 0.64H) 7.22-7.93 (m, 4H); EIMS found M⁺: 347.1287, 349.1256. C₁₉H₂₂ClNO₃ requires 347.1288, 349.1258.

### EXAMPLE 23

### 4-(2-Chlorophenyl)-6-(3-methoxypropyl)-1,3-dioxo-1,2,3,3a,4,5,6,8b-octahydro-2,6-diaza-as-indacene-7-carboxylic acid ethyl ester

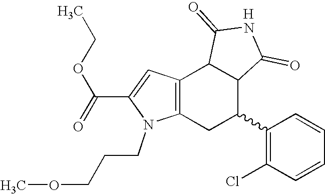

A mixture of 5-[2-(2-chlorophenyl)vinyl]-1-(3-methoxypropyl)-1H-pyrrole-2-carboxylic acid ethyl ester (1.85 g, 5.33 mmol) prepared as described in Example 22 and maleimide (0.78 g, 8.00 mmol) was heated at 180-210° C. (bath temperature) for 6 h. After cooling to room temperature the residue was dissolved in ethyl acetate. The ethyl acetate solution was washed with water several times, dried (Na₂SO₄) and evaporated to give 4-(2-chlorophenyl)-6-(3-methoxypropyl)-1,3-dioxo-1,2,3,3a,4,5,6,8b-octahydro-2,6-diaza-as-indacene-7-carboxylic acid ethyl ester (2.37 g, 100%) as a light orange oil; API-CI Found: 445.0 (M+1, 100), 447.0 (33); 443.0 (M−1,100), 445.0 (33). This material was used for the next step without further purification.

### EXAMPLE 24

### 2-[2-(2-Methoxyphenyl)vinyl]-1H-pyrrole

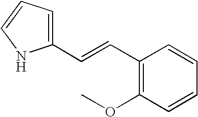

2-[2-(2-Methoxyphenyl)vinyl]-1H-pyrrole was prepared from pyrrole-2-carboxaldehyde (10.00 g, 60.0 mmol) and (2-chlorobenzyl)triphenylphosphonium chloride (25.06 g, 60.0 mmol), using the procedure described in Example 11 to give 10.0 g of the title compound as a white solid, melting point (uncorrected), 142.4-142.7° C.; ¹H NMR (400 MHz, DMSO-D6) 8 ppm 3.8 (s, 3H) 6.0 (m, 1H) 6.2 (t, J=3.5 Hz, 1H) 6.8 (m, 1H) 6.9 (t, J=7.4 Hz, 1H) 7.0 (m, 3H) 7.2 (m, 1H) 7.5 (dd, J=7.6, 1.5 Hz, 1H) 11.2 (s, 1H).

### EXAMPLE 25

### 1-[2-(tert-Butyldimethylsilanyloxy)-ethyl]-2-[2-(2-methoxyphenyl)-vinyl]-1H-pyrrole

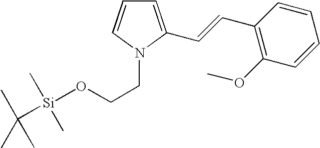

1-[2-(tert-Butyldimethylsilanyloxy)ethyl]-2-[2-(2-methoxyphenyl)vinyl]-1H-pyrrole was prepared from 2-[2-(2-methoxyphenyl)vinyl]-1H-pyrrole (5.00 g, 25.10 mmol), prepared as described in Example 24, and (2-bromoethoxy)-tert-butyldimethylsilane (6.60 g, 27.6 mmol), using the procedure described in Example 10 to give 8.17 g of the title compound as a yellow oil; ¹H NMR (400 MHz, DMSO-D6) 8 ppm −0.1 (m, 6H) 0.8 (m, 9H) 3.8 (t, J=5.2 Hz, 2H) 3.8 (s, 3H) 4.1 (t, J=5.4 Hz, 2H) 6.0 (m, 1H) 6.4 (dd, J=3.7, 1.5 Hz, 1H) 6.8 (m, 1H) 6.9 (t, J=7.4 Hz, 1H) 7.0 (d, J=8.3 Hz, 1H) 7.1 (t, J=16.5 Hz, 2H) 7.2 (m, 1H) 7.6 (dd, J=7.7, 1.6 Hz, 1H); LCMS (Phenomenex Develosil Combi RP3 50×4.6 mm column, gradient method 10-2% acetonitrile/water eluent (0.1% formic acid) in 2.0 min with 2 min hold, MSD in ESI positive mode) 99% at 2.29 min (UV_{254 nm}); API-ES 358.2 (100), 359.2 (30).

### EXAMPLE 26

### 6-[2-(tert-Butyldimethylsilanyloxy)ethyl]-4-(2-methoxyphenyl)-4,5,6,8b-tetrahydro-3aH-2,6-diaza-as-indacene-1,3-dione

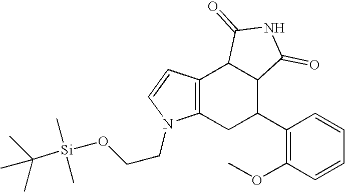

6-[2-(tert-Butyldimethylsilanyloxy)ethyl]-4-(2-methoxyphenyl)-4,5,6,8b-tetrahydro-3aH-2,6-diaza-as-indacene-1,3-dione was prepared from 1-[2-(tert-butyldimethylsilanyloxy)ethyl]-2-[2-(2-methoxyphenyl)vinyl]-1H-pyrrole (8.17 g, 22.8 mmol) and maleimide (3.33 g, 22.8 mmol), using the procedure described in Example 12 to give 8.32 g of the title compound as a yellow solid; ¹H NMR (400 MHz, CDCl₃) δ ppm −0.1 (m, 6H) 0.8 (m, 9H) 1.6 (s, 2H) 2.8 (dd, J=15.0, 4.3 Hz, 1H) 3.1 (m, 1H) 3.6 (ddd, J=12.8, 3.9, 3.8 Hz, 1H) 3.8 (m, 2H) 3.8 (s, 3H) 3.9 (t, J=5.9 Hz, 2H) 4.1 (d, J=7.8 Hz, 1H) 6.3 (d, J=2.7 Hz, 1H) 6.6 (d, J=2.9 Hz, 1H) 6.9 (m, 1H) 7.0 (td, J=7.6, 1.0 Hz, 1H) 7.3 (m, 3H) 7.5 (dd, J=7.6, 1.5 Hz, 1H); melting point (uncorrected), 153.3-155.4° C.

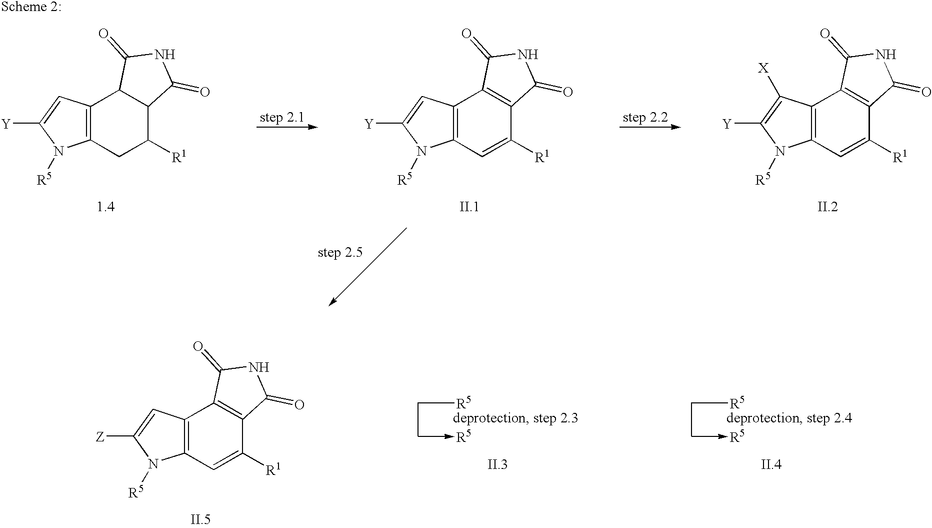

### Procedures for Scheme 2

### EXAMPLE 27

### 4-(2-Methoxyphenyl)-6-methyl-6H-2,6-diaza-as-indacene-1,3-dione

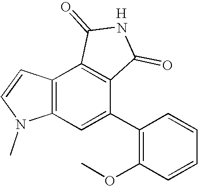

A solution of 4-(2-methoxyphenyl)-6-methyl-4,5,6,8b-tetrahydro-3aH-2,6-diaza-as-indacene-1,3-dione (0.50 g, 1.61 mmol) prepared as described in Example 5 and DDQ (0.80 g, 3.52 mmol) in p-dioxane (30 mL) was refluxed for 2 h. A further quantity of DDQ (0.10 g, 0.44 mmol) was added and refluxing was continued for a further 1 h. The solution was diluted with ethyl acetate and washed with saturated aqueous sodium hydrogencarbonate solution until the washings were colorless. The ethyl acetate solution was worked up and chromatographed on silica. Elution with ethyl acetate/petroleum ether (2:3) gave the title compound (0.26 g, 52%), which crystallised from tetrahydrofuran/petroleum ether as a yellow powder, mp 243-245° C. ¹H NMR (400 MHz, DMSO-D6) δ ppm 3.66 (s, 3H), 3.88 (s, 3H), 6.81 (dd, J=3.1, 0.8 Hz, 1H), 7.02 (dt, J=7.4, 0.9 Hz, 1H), 7.07 (br d, J=8.2 Hz, 1H), 7.28 (dd, J=7.4, 1.7 Hz, 1H), 7.36-7.41 (m, 1H), 7.62 (d, J=0.8 Hz, 1H), 7.71 (d, J=3.1 Hz, 1H), 10.70 (br s, 1H). API-CI Found 307 (M+1). Elemental Analysis for C₁₈H₁₄N₂O₃. % C (calc/found) 70.58/70.57, % H 4.61/4.52, % N 9.14/9.09.

### EXAMPLE 28

### 8-Bromo-4-(2-methoxyphenyl)-6-methyl-6H-2,6-diaza-as-indacene-1,3-dione

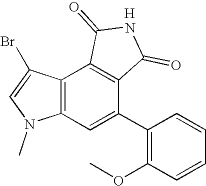

Bromine (5.8 μL, 0.11 mmol) was added dropwise at −60° C. to a stirred solution of 4-(2-methoxyphenyl)-6-methyl-6H-2,6-diaza-as-indacene-1,3-dione (35 mg, 0.11 mmol), prepared as described in Example 27 in CHCl₃ (25 mL). After 30 min. at this temperature a solution of 1N aqueous sodium thiosulfate was added and the mixture was warmed to room temperature and diluted with ethyl acetate. After washing with water the organic solution was dried over Na₂SO₄ and concentrated in vacuo to give title compound. This crystallised from tetrahydrofuran/petroleum ether as a yellow powder (28.2 mg, 64%), mp 216-220° C. ¹H NMR (400 MHz, DMSO-D6) δ ppm 3.65 (s, 3H), 3.87 (s, 3H), 7.02 (dt, J=7.4, 0.9 Hz), 7.06 (br d, J=8.2 Hz), 1H), 7.27 (dd, J=7.4, 1.7 Hz), 7.37-7.42 (m, 1H), 7.70 (s, 1H), 7.90 (s, 1H), 10.80 (br s, 1H). API-CI Found: 0.384, 386 (M−1). Elemental Analysis for C₁₈H₁₃BrN₂O₃.¼H₂O, % C (calc/found) 55.47/55.80, % H 3.49/3.39, % N 7.19/6.98.

### EXAMPLE 29

### 4-(2-Chlorophenyl)-6-methyl-6H-2,6-diaza-as-indacene-1,3-dione

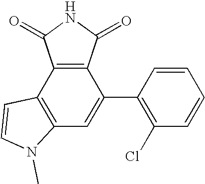

To a solution of 4-(2-chlorophenyl)-6-methyl-4,5,6,8b-tetrahydro-3aH-2,6-diaza-as-indacene-1,3-dione prepared as described in Example 3 in dioxane (400 mL) was added MnO₂ (81 g) and the reaction mixture was heated to reflux for 4 hours. The reaction mixture was cooled to room temperature, filtered through celite to remove the MnO₂, and concentrated to dryness. The product was columned using a stepwise gradient 2%, 4%, 6%, 10% and 15% ethyl acetate in hexanes to give the title compound (4.1 g); The title compound was further purified by trituration with diethyl ether (1.74 g) or column chromatography using a gradient of acetone (5% to 100%) in hexanes (0.7 g);

¹H NMR (400 MHz, DMSO-D6) □ ppm 3.89 (s, 3H), 6.83 (dd, J=3.2, 1.0 Hz, 1H), 7.44 (m, 3H), 7.53 (m, 1H), 7.70 (s, 1H) 7.77 (d, J=2.9 Hz, 1H) 10.87 (s, 1H); APCI− 389,387

### EXAMPLE 30

### 8-Chloro-4-(2-methoxyphenyl)-6-methyl-6H-2,6-diaza-as-indacene-1,3-dione

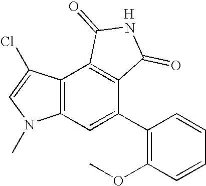

A solution of N-chlorosuccinimide (46 mg, 0.34 mmol) in DMF (0.5 mL) was added at room temperature to a solution of 4-(2-methoxyphenyl)-6-methyl-6H-2,6-diaza-as-indacene-1,3-dione (0.10 g, 0.33 mmol), prepared as described in Example 27, in DMF (5 mL) and the solution was stirred for 18 h. After dilution with ethyl acetate it was washed well with water, then brine and dried over Na₂SO₄. The solvent was removed in vacuo to give crude product which crystallised from tetrahydrofuran/petroleum ether as a yellow powder (90.8 mg, 88%), mp 267-271° C. ¹H NMR (400 MHz, DMSO-D6) δ ppm 3.65 (s, 3H), 3.86 (s, 3H), 7.02 (dt, J=7.4, 0.9 Hz, 1H), 7.07 (br d, J=7.8 Hz 1H), 7.28 (dd, J=7.4, 1.7 Hz, 1H), 7.36-7.42 (m, 1H), 7.70 (s, 1H), 7.87 (s, 1H), 10.81 (br, 1H). API-CI Found: 341, 343 (M+1). Elemental Analysis for C₁₈H₁₃ClN₂O₃, % C (calc/found) 63.44/63.33, % H 3.84/3.95, % N 8.22/8.29.

### EXAMPLE 31

### 8-Bromo-6-methyl-4-(2-methylsulfanylphenyl)-6H-2,6-diaza-as-indacene-1,3-dione

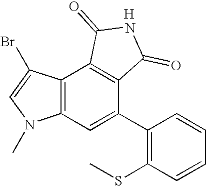

Reaction of the sulfide 6-methyl-4-(2-methylsulfanylphenyl)-6H-2,6-diaza-as-indacene-1,3-dione prepared as described in Example 134 with bromine at −60° C. as described in Example 28 gave a solid which was chromatographed on silica. Elution with ethyl acetate/petroleum ether (1:1) gave 8-bromo-6-methyl-4-(2-methylsulfanylphenyl)-6H-2,6-diaza-as-indacene-1,3-dione (67%), which crystallised from ethyl acetate/petroleum ether as a yellow powder, mp 240-243° C. ¹H NMR (400 MHz, DMSO-D6) δ ppm 2.30 (s, 3H), 3.86 (s, 3H), 7.23-7.25 (m, 2H), 7.36 (br d, J=7.9 Hz, 1H), 7.39-7.45 (m, 1H), 7.71 (s, 1H), 7.93 (s, 3H), 10.86 (br s, 1H). FABMS found: [M+H]⁺ 400.9959, 402.9950. C₁₈H₁₄BrN₂SO₂ requires 400.9959, 402.9939.

### EXAMPLE 32

### 4-(2-Chlorophenyl)-8-iodo-6-methyl-6H-2,6-diaza-as-indacene-1,3-dione

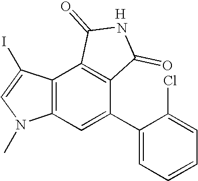

A solution of ICl (55 mg, 0.33 mmol) in CHCl₃ (5 mL) was added dropwise at room temperature to a solution of 4-(2-chlorophenyl)-6-methyl-6H-2,6-diaza-as-indacene-1,3-dione (0.10 g, 0.32 mmol), prepared as described in Example 29 in CHCl₃ (100 mL). After 2 h a further quantity (10 mg) of ICl was added and the solution was stirred for a further 1 h. 2N aqueous Na₂SO₃ solution (10 mL) was added and after 15 min the mixture was diluted with water and the aqueous layer was removed. The organic solution was worked up to give an oil which was chromatographed on silica. Elution with ethyl acetate/petroleum ether (15:35) gave the title compound (73 mg, 52%) which crystallised from diethyl ether/petroleum ether as a bright yellow solid, mp 150-154° C. (dec.). ¹H NMR (400 MHz, DMSO-D6) δ ppm 3.89 (s, 3H), 7.41-7.48 (m, 3H), 7.52-7.55 (m, 1H), 7.75 (s, 1H), 7.95 (s, 1H), 10.88 (br s, 1H). FABMS found: [M+H]+436.9553, 438.9521. C₁₇H₁₁ClIN₂O₂ requires 436.9554, 438.9524.

### EXAMPLE 33

### 4-(2-Chloro-6-methoxyphenyl)-6-methyl-6H-2,6-diaza-as-indacene-1,3-dione

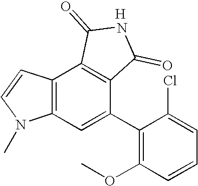

Treatment of the 4-(2-chloro-6-methoxyphenyl)-6-methyl-4,5,6,8b-tetrahydro-3aH-2,6-diaza-as-indacene-1,3-dione prepared as described in Example 7 with MnO₂ in p-dioxane as described in Example 29 gave a solid which was chromatographed on silica. Elution with ethyl acetate/petroleum ether (1:4) gave 4-(2-chloro-6-methoxyphenyl)-6-methyl-6H-2,6-diaza-as-indacene-1,3-dione (32%), which crystallised from ethyl acetate/petroleum ether as a pale yellow powder, mp 268-272° C. ¹H NMR (400 MHz, DMSO-D6) δ ppm 3.65 (s, 3H), 3.88 (s, 3H), 6.83 (dd, J=3.1, 0.8 Hz, 1H), 7.09 (dd, J=8.5, 0.9 Hz, 1H), 7.14 (dd, J=8.5, 0.9 Hz, 1H), 7.41 (t, J=8.5 Hz, 1H), 7.63 (d, J=0.8 Hz, 1H), 7.75 (d, J=3.1 Hz, 1H), 10.77 (br, 1H). API-CI Found: 341, 343 (M+1).

### EXAMPLE 34

### 8-Bromo-4-(2-chloro-6-methoxyphenyl)-6-methyl-6H-2,6-diaza-as-indacene-1,3-dione

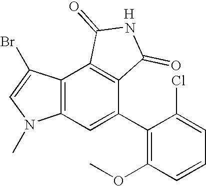

Reaction of 4-(2-chloro-6-methoxyphenyl)-6-methyl-6H-2,6-diaza-as-indacene-1,3-dione) prepared as described in Example 33 with bromine at −60° C. as described in Example 28 gave a solid which was chromatographed on silica. Elution with ethyl acetate/petroleum ether (1:1) gave 8-bromo-4-(2-chloro-6-methoxyphenyl)-6-methyl-6H-2,6-diaza-as-indacene-1,3-dione (78%), which crystallised from ethyl acetate/petroleum ether as a yellow powder, mp 238-242° C. ¹H NMR (400 MHz, DMSO-D6) δ ppm 3.65 (s, 3H), 3.86 (s, 3H), 7.10 (dd, J=8.5, 0.9 Hz, 1H), 7.15 (dd, J=8.5, 0.9 Hz, 1H), 7.41 (t, J=8.5 Hz, 1H), 7.73 (s, 1H), 7.94 (s, 1H), 10.87 (br s, 1H). Elemental Analysis for C₁₈H₁₂BrClN₂O₃% C (calc/found) 51.52/51.69, % H 2.88/3.20, % N 6.67/6.51.

### EXAMPLE 35

### 4-(2-Chlorophenyl)-6-[2-(2-ethoxyethoxy)ethyl]-6H-2,6-diaza-as-indacene-1,3-dione

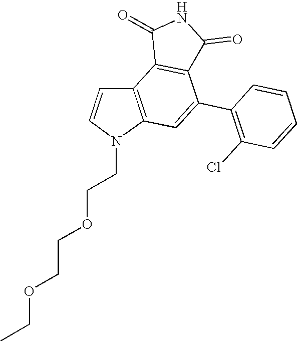

Aromatisation of 4-(2-chlorophenyl)-6-[2-(2-ethoxyethoxy)ethyl]-4,5,6,8b-tetrahydro-3aH-2,6-diaza-as-indacene-1,3-dione, prepared as described in Example 9 with MnO₂ as described in Example 29 gave 4-(2-chlorophenyl)-6-[2-(2-ethoxyethoxy)ethyl]-6H-2,6-diaza-as-indacene-1,3-dione which was chromatographed on silica. Elution with ethyl acetate/petroleum ether (1:9) gave the product (34%), which was triturated with diethyl ether to leave a yellow powder, mp 173° C. ¹H NMR (400 MHz, DMSO-D6) δ ppm 0.98 (t, J=7.0 Hz, 3H), 3.28 (q, J=7.0 Hz, 2H), 3.30-3.40 (m), 3.47 (m, 2H), 3.74 (t, J=5.1 Hz, 2H), 4.46 (t, J=5.0 Hz, 2H), 6.85 (dd, J=3.1, 0.6 Hz, 1H), 7.38-7.48 (m, 3H), 7.52-7.56 (m, 1H), 7.76 (d, J=0.6 Hz, 1H), 7.80 (d, J=3.1 Hz, 1H), 10.81 (br, 1H). API-CI Found; 413, 415 (M+1). Elemental Analysis for C₂₂H₂₁ClN₂O₄.1.2H₂O, % C (calc/found) 62.63/62.54, % H 5.25/4.94, % N 6.64/6.60.

### EXAMPLE 36

### 8-Bromo-4-(2-chlorophenyl)-6-[2-(2-ethoxyethoxy)ethyl]-6H-2,6-diaza-as-indacene-1,3-dione

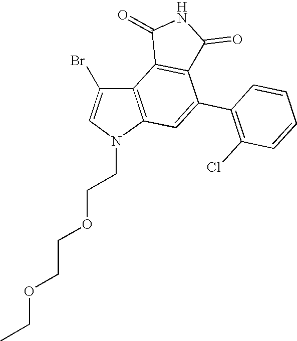

Bromination of 4-(2-chlorophenyl)-6-[2-(2-ethoxyethoxy)ethyl]-6H-2,6-diaza-as-indacene-1,3-dione prepared as described in Example 35 using the procedure described in Example 27 gave the title compound which crystallised from ethyl acetate/petroleum ether as a yellow powder (93%), mp 108-110° C. ¹H NMR (400 MHz, DMSO-D6) δ ppm 0.99 (t, J=7 Hz, 3H), 3.28 (q, J=7 Hz, 2H), 3.30-3.40 (m), 3.43-3.50 (m, 2H), 3.73 (t, J=5.0 Hz, 2H), 4.46 (t, J=4.7 Hz, 2H), 7.38-7.48 (m, 3H), 7.52-7.56 (m, 1H), 7.85 (s, 1H), 7.96 (s, 1H), 10.91 (br s, 1H). API-CI Found: 491, 493, 495 (M+1). Elemental Analysis for C₂₂H₂₀BrClN₂O₄, % C (calc/found) 53.73/53.60, % H 4.10/3.91, % N 5.70/5.77.

### EXAMPLE 37

### 8-Bromo-4-(2-chlorophenyl)-6-methyl-6H-2,6-diaza-as-indacene-1,3-dione

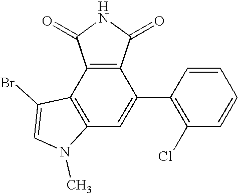

To a solution of compound 4-(2-chlorophenyl)-6-methyl-6H-2,6-diaza-as-indacene-1,3-dione prepared as described in Example 29 in tetrahydrofuran (1 mL) was added NBS (34 mg). The reaction mixture was stirred at room temperature for 30 minutes and then poured into ethyl acetate (25 mL). The organic components were washed with water (2×10 mL) and saturated sodium chloride (10 mL), dried over magnesium sulphate and concentrated to dryness to yield the title compound (28 mg); ¹H NMR (400 MHz, DMSO-D6) δ ppm 3.88 (s, 3H) 7.44 (m, 3H) 7.52 (m, 1H) 7.79 (s, 1H) 7.97 (s, 1H) 10.97 (brs, 1H); APCI− 391, 389, 388, 387.

### EXAMPLE 38

### 4-(2-Chlorophenyl)-6-(2-methoxyethyl)-6H-2,6-diaza-as-indacene-1,3-dione

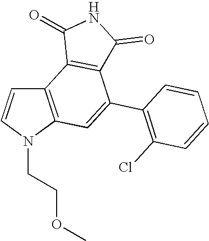

To a solution of 4-(2-chlorophenyl)-6-(2-methoxyethyl)-4,5,6,8b-tetrahydro-3aH-2,6-diaza-as-indacene-1,3-dione (0.75 g) prepared as described in Example 12 in nitrobenzene (30 mL) was added 10% Pd/C (1.5 g), and the mixture was heated at 175° C. overnight. The reaction mixture was cooled and filter through a short pad of Celite, which was as washed with nitrobenzene (3×10 mL). Hexane was added to the nitrobenzene until the total volume was 500 mL. The solid precipitate was collect and dried under vacuum at 50° C. to give the title compound (0.64 g). ¹H NMR (400 MHz, DMSO-D6) δ ppm 3.19 (s, 3H), 3.64 (t, J=5.1 Hz, 2H) 4.46 (t, J=5.0 Hz, 2H) 6.84 (d, J=3.2 Hz, 1H), 7.42 (m, 3H) 7.45 (m, 1H), 7.69 (m, 1H), 7.78 (m, 2H), 10.86 (s, 1H). Mass spectrum APCI−, MH− 355,353.

### EXAMPLE 39

### 8-Bromo-4-(2-chlorophenyl)-6-(2-methoxyethyl)-6H-2,6-diaza-as-indacene-1,3-dione

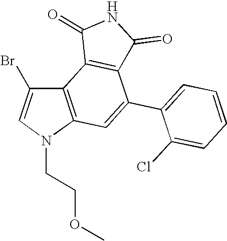

To a solution of 4-(2-chlorophenyl)-6-(2-methoxyethyl)-6H-2,6-diaza-as-indacene-1,3-dione (0.4 g) prepared as described in Example 38, in chloroform (400 mL) at −56° C. was added dropwise a solution of bromine in chloroform (0.43 g in 10 mL). After 30 min saturated aqueous sodium metabisulfite (50 mL) was added and the reaction mixture was warmed to room temperature. The aqueous phase was extracted with chloroform (2×100 mL), the combined organic phase was dried (MgSO₄) and concentrated to dryness. Purification by column chromatography on silica using a gradient of 20% to 75% ethyl acetate in hexane gave the title compound (0.23 g); ¹H NMR (400 MHz, DMSO-D6) δ ppm 3.33 (s, 3H), 3.63 (t, J=5.0 Hz, 2H), 4.46 (t, J=5.0 Hz, 2H), 7.43 (m, 3H), 7.53 (m, 1H), 7.87 (s, 1H), 7.95 (s, 1H), 10.96 (s, 1H). APCI− (MH− 435,343,433,431).

### EXAMPLE 40

### 8-Bromo-4-(2-chlorophenyl)-6-(2-hydroxyethyl)-6H-2,6-diaza-as-indacene-1,3-dione

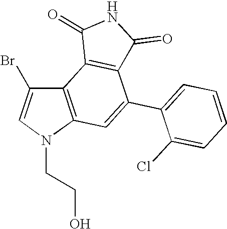

To a solution of 8-Bromo-4-(2-chlorophenyl)-6-(2-methoxy-ethyl)-6H-2,6-diaza-as-indacene-1,3-dione (0.1 g), prepared as described in Example 39, in dichloromethane (4 mL) was added a 2M solution of boron tribromide in dichloromethane (0.5 mL). After 4 hours, saturated aqueous sodium bicarbonate solution (25 mL) was added and the organic components were extracted into ethyl acetate (3×25 mL). Purification by column chromatography on silica using a gradient of 20% to 75% ethyl acetate in hexane, then crystallisation by vapor diffusion using tetrahydrofuran and hexane yielded the title compound (60 mg), ¹H NMR (400 MHz, DMSO-D6 δ ppm 3.59 (t, J=5.3 Hz, 1H), 4.35 (t, J=5.2 Hz, 1H), 4.92 (m, 1H), 7.43 (m, 3H), 7.53 (m, 1H), 7.84 (s, 1H), 7.95 (s, 1H), 10.96 (s, 1H); elemental analysis for C₁₂H₁₈F₃NO₃.0.21 THF: % C (calc/found) 52.04/52.42, % H 3.17/3.23, % N 6.44/6.08.

### EXAMPLE 41

### 6-[3-(tert-Butyldimethylsilanyloxy)propyl]4-(2-chlorophenyl)-6H-2,6-diaza-as-indacene-1,3-dione

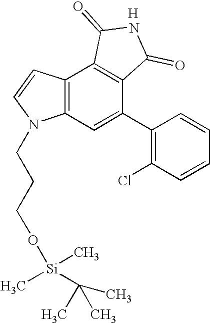

To a mixture of 2-[2-(2-chlorophenyl)vinyl]-1H-pyrrole (0.83 g, 4.09 mmol), prepared as described in Example 14 and 1-bromo-3(tert-butyldimethylsilanyloxy)propane (1.14 g, 4.50 mmol) at 20° C. (under N₂) was added NaH (118 mg, 4.91 mmol) and the reaction mixture was further stirred for 1 h. It was partitioned between ethyl acetate and brine. The ethyl acetate solution was further washed with water, dried (Na₂SO₄) and evaporated to give a brown oil which was treated with maleimide (0.60 g, 6.15 mmol) at 180-210° C. for 3 h. After cooling to room temperature, the material was dissolved in DMF and poured into water. The precipitate was filtered, washed with water and dried. The crude product was treated with MnO₂ (2×3 g) in p-dioxane (20 mL) at reflux for 20 h. The reaction was filtered through Celite and the filtrate evaporated to dryness. The crude product was purified by a silica column (chloroform/methanol=20:1) to give the title compound (0.90 g, 47%) as an orange solid; mp 158-160° C.; ¹H NMR (400 MHz, DMSO-D6) □ ppm −0.05 & −0.04 (2s, 6H) 0.79 (s, 9H) 1.94 (m, 2H) 3.53 (t, J=6.1 Hz, 2H) 3.50 (m, 2H) 6.87 (dd, J=3.1 & 0.7 Hz, 1H) 7.38-7.55 (m, 4H) 7.69 (d, J=0.7 Hz, 1H) 7.80 (d, J=3.1 Hz, 1H) 10.83 (s, 1H); elemental Analysis for C₂₅H₂₉ClN₂O₃Si: % C (calc/found) 64.02/63.78, % H 6.23/6.32, % N 5.97/5.94.

### EXAMPLE 42

### 4-(2-Chlorophenyl)-6-(3-hydroxypropyl)-6H-2,6-diaza-as-indacene-1,3-dione

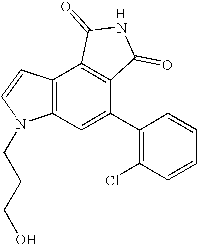

6-[3-(tert-Butyldimethylsilanyloxy)propyl]-4-(2-chlorophenyl)-6H-2,6-diaza-as-indacene-1,3-dione (80 mg, 0.171 mmol), prepared as described in Example 41 was stirred with 2N H₂SO₄ (2 mL) in p-dioxane (6 mL) at 20° C. for 45 min. Partitioning between ethyl acetate and water, and evaporation of the organic solvent gave the title compound (50 mg, 83%) as a yellow solid; mp 225-227° C.; ¹H NMR (400 MHz, DMSO-D6) δ ppm 1.90 (m, 2H) 3.37 (m, 2H) 4.36 (t, J=7.0 Hz, 2H) 4.59 (t, J=5.0 Hz, exchangeable with D₂O, 1H) 6.86 (dd, J=3.1 & 0.7 Hz, 1H) 7.40-7.57 (m, 4H) 7.72 (d, J=0.7 Hz, 1H) 7.81 (d, J=3.1 Hz, 1H) 10.82 (s, exchangeable with D₂O, 1H); FABMS found [M+H]⁺: 355.0844, 357.0824. C₁₉H₁₆ClN₂O₃ requires 355.0849, 357.0820.

### EXAMPLE 43

### 4-(2-Chlorophenyl)-6-(3-methoxypropyl)-6H-2,6-diaza-as-indacene-1,3-dione

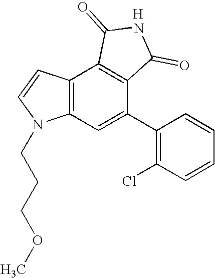

A mixture of 4-(2-chlorophenyl)-6-(3-methoxypropyl)-4,5,6,8b-tetrahydro-3aH-2,6-diaza-as-indacene-1,3-dione (2.84 g, 7.63 mmol), prepared as described in Example 16 and MnO₂ (5 g) in p-dioxane (50 mL) was stirred at reflux for 23 h. Another batch of MnO₂ (3 g) was added and refluxing was continued for 5 h. The hot mixture was filtered through Celite and the residue washed with dichloromethane several times. The combined filtrates was evaporated and columned (silica gel; ethyl acetate/petroleum ether=1:5) to give the title compound (1.51 g, 54%) as an orange solid; mp 171-172° C.; API-CI Found: 369.0 (M+1, 100), 371.0 (35); ¹H NMR (400 MHz, DMSO-D6) δ ppm 1.99 (m, 2H) 3.17 (s, 3H) 3.24 (t, J=6.1 Hz, 2H) 4.34 (t, J=6.9 Hz, 1H) 6.87 (dd, J=3.2 & 0.7 Hz, 2H) 7.39-7.57 (m, 4H) 7.70 (d, J=0.7 Hz, 1H) 7.80 (d, J=3.2 Hz, 1H) 10.83 (s, 1H); FABMS found [M+H]⁺: 369.0991, 371.0898. C₂₀H₁₈ClN₂O₃ requires 369.1006, 371.0976.

### EXAMPLE 44

### 8-Bromo-4-(2-chlorophenyl)-6-(3-methoxypropyl)-6H-2,6-diaza-as-indacene-1,3-dione

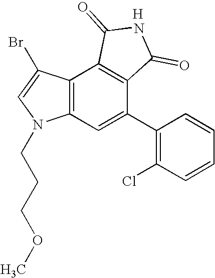

4-(2-Chlorophenyl)-6-(3-methoxypropyl)-6H-2,6-diaza-as-indacene-1,3-dione (1.32 g, 3.58 mmol), prepared as described in Example 43 was treated dropwise with bromine (0.58 g, 3.64 mmol) in chloroform (40 mL) at −60 to −70° C. (chloroform/dry-ice bath) for 1 h. Excess aqueous sodium thiosulfate solution was added, followed by dichloromethane and the mixture was stirred at 0° C. for 15 nin. The organic layer was separated, washed with water, dried (Na₂SO₄), evaporated and the residue columned (silica gel; dichloromethane/ethyl acetate=50:1) to give the title compound (1.50 g, 94%) as a pale yellow solid; mp 149-151° C. ¹H NMR (400 MHz, DMSO-D6) δ ppm 1.98 (m, 2H) 3.16 (s, 3H) 3.24 (t, J=6.1 Hz, 2H) 4.34 (t, J=6.9 Hz, 2H) 7.40-7.48 (m, 3H) 7.52-7.56 (m, 1H) 7.79 (s, 1H) 8.00 (s, 1H) 10.93 (s, 1H); FABMS found [M+H]⁺: 447.0107, 449.0093 451.0080. C₂₀H₁₇BrClN₂O₃ requires 447.0111, 449.0090 (449.0081), 451.0061; Elemental Analysis for C₂₀H₁₆ClN₂O₃ 0.3 hexane, % C (calc/found) 55.29/55.54, % H 4.30/4.28, % N 5.92/6.01.

### EXAMPLE 45

### 8-Bromo-6-(3-bromopropyl)-4-(2-chlorophenyl)-6H-2,6-diaza-as-indacene-1,3-dione

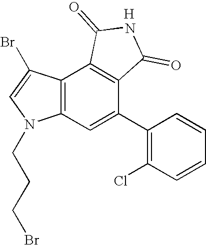 and

### 8-Bromo-4-(2-chlorophenyl)-6-(3-hydroxypropyl)-6H-2,6-diaza-as-indacene-1,3-dione

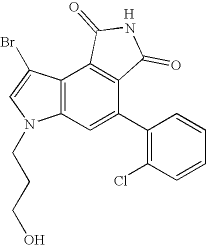

A solution of 8-bromo-4-(2-chlorophenyl)-6-(3-methoxypropyl)-6H-2,6-diaza-as-indacene-1,3-dione (1.22 g, 2.72 mmol) prepared as described in Example 44 in dichloromethane (30 mL) was treated with BBr₃ (1M in dichloromethane) (5.0 mL) at 20° C. for 6 h. After partitioning between dichloromethane and water the organic solution was dried (Na₂SO₄), evaporated and separated by a column (silica gel; chloroform/methanol=20:1) into (i) 8-bromo-6-(3-bromopropyl)-4-(2-chlorophenyl)-6H-2,6-diaza-as-indacene-1,3-dione (0.20 g, 14%) as an orange solid; mp 121-123° C.; ¹H NMR (400 MHz, DMSO-D6) δ ppm 2.30 (m, 2H) 3.47 (t, J=6.6 Hz, 2H) 4.41 (t, J=6.9 Hz, 2H) 7.40-7.48 (m, 3H) 7.52-7.57 (m, 1H) 7.89 (s, 1H) 8.03 (s, 1H) 10.90 (s, 1H); FABMS found [M+H]⁺: 494.9101, 496.9101 498.9091, 500.9072. C₁₉H₁₄Br₂ClN₂O₂ requires 494.9110, 496.9090 (496.9081), 498.9069 (498.9060), 500.9040; and (ii) 8-Bromo-4-(2-chlorophenyl)-6-(3-hydroxypropyl)-6H-2,6-diaza-as-indacene-1,3-dione (1.01 g, 85%) as a yellow solid; mp 155-157° C.; ¹H NMR (400 MHz, DMSO-D6) δ ppm 1.89 (m, 2H) 3.36 (m, 2H) 4.34 (t, J=7.0 Hz, 2H) 4.58 (t, J=5.0 Hz, 1H) 7.39-7.48 (m, 3H) 7.52-7.56 (m, 1H) 7.82 (s, 1H) 7.99 (s, 1H) 10.92 (s, exchangeable with D₂O, 1H); FABMS found [M+H]⁺: 432.9938, 434.9927, 436.9902. C₁₉H₁₅BrClN₂O₃ requires 432.9954, 434.9925 (434.9934), 436.9904.

### EXAMPLE 46

### 4-(2-Chlorophenyl)-6-(3-methoxypropyl)-1,3-dioxo-1,2,3,6-tetrahydro-2,6-diaza-as-indacene-7-carboxylic acid ethyl ester

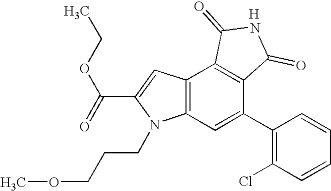

A mixture of 4-(2-chlorophenyl)-6-(3-methoxypropyl)-1,3-dioxo-1,2,3,3a,4,5,6,8b-octahydro-2,6-diaza-as-indacene-7-carboxylic acid ethyl ester (2.37 g, 5.33 mmol), prepared as described in Example 23 and MnO₂ (2×8 g) in p-dioxane (300 mL) was stirred at reflux for 10 h. The mixture was filtered through Celite and the residue washed with ethyl acetate several times. The combined filtrates were evaporated and columned (silica gel; chloroform/methanol=10:1) to give the title compound (0.98 g, 42%) as an orange solid; mp 235-237° C.; API-CI Found: 441.0 (M+1, 100), 443.0 (30); 439.0 (M−1, 100%), 441.0 (33); ¹H NMR (400 MHz, DMSO-D6) 8 ppm 1.39 (t, J□7.1 Hz, 3H) 1.96 (m, 2H) 3.11 (s, 3H) 3.24 (t, J=6.0 Hz, 2H) 4.39 (t, J=7.1 Hz, 2H) 4.71 (t, J=6.9 Hz, 2H) 7.42-7.51 (m, 3H) 7.54-7.59 (m, 2H) 7.86 (d, J=0.4 Hz, 1H) 11.03 (s, 1H); Elemental Analysis for C₂₃H₂₁ClN₂O₅, % C (calc/found) 62.66/62.58, % H 4.80/4.65, % N 6.35/6.57.

### EXAMPLE 47

### 4-(2-Chlorophenyl)-6-(3-methoxypropyl)-1,3-dioxo-1,2,3,6-tetrahydro-2,6-diaza-as-indacene-7-carboxylic acid

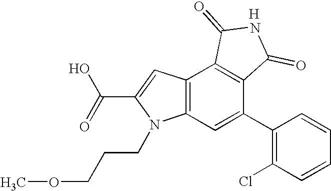

A mixture of 4-(2-chlorophenyl)-6-(3-methoxypropyl)-1,3-dioxo-1,2,3,6-tetrahydro-2,6-diaza-as-indacene-7-carboxylic acid ethyl ester (132 mg, 0.30 mmol), prepared as described in Example 46, potassium tert-butoxide (168 mg, 1.50 mmol) and tert-butanol (5 g) was stirred at 30° C. (bath temperature) until the solid tert-butanol melted. The reaction was then stirred at 20° C. If the mixture solidified, the temperature was raised up to 30° again to melt it. This procedure was repeated as required. After total reaction time of 5 h the reaction flask was immersed into an ice-bath and ice-water was added. After acidification with 5M HCl to pH<1 the resulting yellow precipitate was filtered, washed with water (3×) and dried to give the title compound (122 mg, 98%); mp 272-274° C.; API-CI Found: 413.0 (M+1, 100), 415.0 (33); 411.0 (M−1, 100%), 413.0 (33); ¹H NMR (400 MHz, DMSO-D6) δ ppm 1.96 (m, 2H) 3.11 (s, 3H) 3.24 (t, J=6.0 Hz, 2H) 4.72 (t, J=6.9 Hz, 2H) 7.42-7.52 (m, 3H) 7.50 (d, J=0.4 Hz, 1H) 7.54-7.59 (m, 1H) 7.82 (d, J=0.4 Hz, 1H) 10.99 (s, exchangeable with D₂O, 1H) 13.5 (br s, exchangeable with D₂O, 1H); Elemental Analysis for C₂₁H₁₇ClN₂O₅, % C (calc/found) 61.10/61.08, % H 4.15/4.44, % N 6.79/7.06.

### EXAMPLE 48

### 6-(3-Bromopropyl)-4-(2-chlorophenyl)-1,3-dioxo-1,2,3,6-tetrahydro-2,6-diaza-as-indacene-7-carboxylic acid ethyl ester

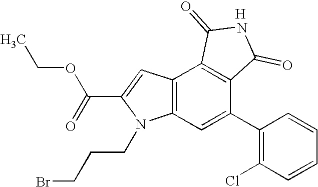 and

### 4-(2-Chlorophenyl)-6-(3-hydroxypropyl)-1,3-dioxo-1,2,3,6-tetrahydro-2,6-diaza-as-indacene-7-carboxylic acid ethyl ester

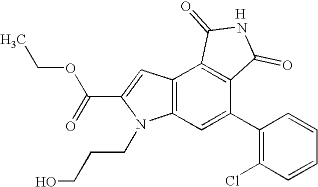

A solution of compound 4-(2-chlorophenyl)-6-(3-methoxypropyl)-1,3-dioxo-1,2,3,6-tetrahydro-2,6-diaza-as-indacene-7-carboxylic acid (0.42 g, 0.96 mmol) prepared as described in Example 47 in dichloromethane (70 mL) was treated with BBr₃ (1M in dichloromethane) (2.0 mL) at 0-5° C. for 67 h. It was partitioned between dichloromethane and water. The organic solution was dried (Na₂SO₄), evaporated to dryness and separated on a column (silica gel; chloroform/methanol=200:1) into (i) bromide 6-(3-bromopropyl)-4-(2-chlorophenyl)-1,3-dioxo-1,2,3,6-tetrahydro-2,6-diaza-as-indacene-7-carboxylic acid ethyl ester (0.11 g, 23%) as an orange solid; mp 234-236° C.; API-CI Found: 488.8 (M+1, 70%), 490.8 (100%), 491.8 (20); ¹H NMR (400 MHz, DMSO-D6) δ ppm 1.40 (t, J=7.1 Hz, 3H) 2.23-2.35 (m, 2H) 3.53 (m, 2H) 4.39 (q, J=7.1 Hz, 2H) 4.76 (t, J=7.3 Hz, 2H) 7.42-7.52 (m, 3H) 7.57 (d, J=0.5 Hz, 1H) 7.51-7.59 (m, 1H) 7.98 (br s, 1H) 11.04 (br s, 1H); Elemental Analysis for C₂₂H₁₈BrClN₂O₄, % C (calc/found) 53.95/54.16, % H 3.71/3.52, % N 5.72/5.71.

and (ii) 4-(2-chlorophenyl)-6-(3-hydroxypropyl)-1,3-dioxo-1,2,3,6-tetrahydro-2,6-diaza-as-indacene-7-carboxylic acid ethyl ester (0.31 g, 76%) as a yellow solid; mp 249-251° C.; API-CI Found: 427.0 (M+1,100%), 429.0 (33); 425.0 (M−1, 55%), 427.0 (18) ¹H NMR (400 MHz, DMSO-D6) δ ppm 1.38 (t, J=7.1 Hz, 3H) 1.84-1.93 (m, 2H) 3.40 (m, 2H) 4.39 (q, J=7.1 Hz, 2H) 4.54 (t, J=5.0 Hz, 1H) 4.70 (t, J=7.1 Hz, 2H) 7.41-7.51 (m, 3H) 7.56 (d, J=0.6 Hz, 1H) 7.54-7.59 (m, 1H) 7.90 (br s, 1H) 11.02 (br s, 1H); Elemental Analysis for C₂₂H₁₉ClN₂O₅, % C (calc/found) 61.90/61.93, % H 4.49/4.42, % N 6.56/6.60.

### EXAMPLE 49

### 6-(3-Bromopropyl)-4-(2-chlorophenyl)-1,3-dioxo-1,2,3,6-tetrahydro-2,6-diaza-as-indacene-7-carboxylic acid

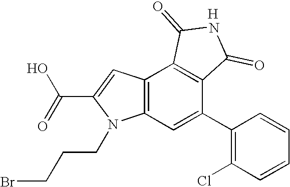

A solution of compound 6-(3-cromopropyl)-4-(2-chlorophenyl)-1,3-dioxo-1,2,3,6-tetrahydro-2,6-diaza-as-indacene-7-carboxylic acid ethyl ester (102 mg, 0.232 mmol), prepared as described in Example 48, in dichloromethane (20 mL) was treated with BBr₃ (1M in dichloromethane) (5.0 mL) at 20° C. for 8 days. It was partitioned between ethyl acetate and water and the organic solution was dried (Na₂SO₄) and evaporated to give the title compound (107 mg, 100%) as a yellow solid; mp 294-295° C.; API-CI Found: 460.8 (M+1, 75%), 462.8 (100%), 464.8 (25); 458.8 (M−1, 75%), 460.8 (100%), 462.8 (20); ¹H NMR (400 MHz, DMSO-D6) δ ppm 2.22-2.36 (m, 2H) 3.48-3.57 (m, 2H) 4.77 (t, J=7.2 Hz, 2H) 7.41-7.51 (m, 3H) 7.52 (d, J=0.4 Hz, 1H) 7.54-7.59 (m, 1H) 7.95 (d, J=0.4 Hz, 1H) 11.02 (s, 1H) 13.50 (br s, 1H); FABMS found [M]+: 460.9908, 462.9885, 464.9880. C₂₁H₁₅BrClN₂O₃ requires 460.9903, 462.9883 (462.9874), 464.9853.

### EXAMPLE 50

### 6-(3-Bromopropyl)-4-(2-chlorophenyl)-7-hydroxymethyl-6H-2,6-diaza-as-indacene-1,3-dione

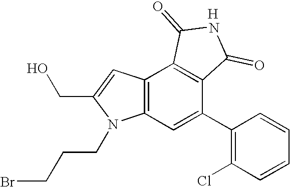

A solution of carboxylic acid 6-(3-bromopropyl)-4-(2-chlorophenyl)-1,3-dioxo-1,2,3,6-tetrahydro-2,6-diaza-as-indacene-7-carboxylic acid (56 mg, 0.121 mmol), prepared as described in Example 49, in THF (5 mL) was reduced with BH₃.SMe₂ (2M in THF) (0.13 mL, 0.26 mmol) at 20° C. for 5 h and then at 5° for 11 days. It was partitioned between ethyl acetate and water. The organic solution was further washed with water, dried (Na₂SO₄), and evaporated to give a yellow solid which was chromatographed on a silica column (chloroform/methanol=50:1) to afford the title compound (21 mg, 39%); mp 218-220° C.; API-CI Found: 446.8 (M+1, 75%), 448.8 (100); ¹H NMR (400 MHz, DMSO-D6) 8 ppm 2.21-2.35 (m, 2H) 3.52-3.61 (m, 2H) 4.40 (t, J=7.4 Hz, 2H) 4.76 (d, J=5.5 Hz, 2H) 5.50 (t, J=5.5 Hz, 1H) 6.83 (br s, 1H) 7.40-7.50 (m, 3H) 7.52-7.58 (m, 1H) 7.73 (d, J=0.5 Hz, 1H) 10.82 (s, 1H); FABMS found [M]+: 447.0117, 449.0081, 451.0086. C₂₀H₁₇BrClN₂O₃ requires 447.0111, 449.0081 (449.0090), 451.0061.

### EXAMPLE 51

### 4-(2-Chlorophenyl)-6-(3-methoxypropyl)-1,3-dioxo-1,2,3,6-tetrahydro-2,6-diaza-as-indacene-7-carboxylic acid amide

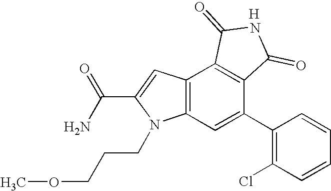

A mixture of 4-(2-chlorophenyl)-6-(3-methoxypropyl)-1,3-dioxo-1,2,3,6-tetrahydro-2,6-diaza-as-indacene-7-carboxylic acid (10 mg, 0.024 mmol), prepared as described in Example 46, POCl₃ (330 mg, 2.15 mmol), and DMF (5 drop) in dichloromethane (5 mL) was stirred at reflux for 45 min. The solution was cooled to 20° C. and excess ammonia gas was bubbled through. The reaction was further stirred at 200 for 10 min, then partitioned between ethyl acetate and cold aq. sodium carbonate solution. The organic layer was separated, dried (Na₂SO₄) and evaporated to give a yellow solid which was recrystallised from dichloromethane/methanol to afford the title compound (5.5 mg, 56%); mp 260-261° C.; API-CI Found: 412.0 (M+1, 100%), 414.0 (33); 410.0 (M−1, 50%), 412.0 (13%); ¹H NMR (400 MHz, DMSO-D6) δ ppm 1.94 (m, 2H) 3.11 (s, 3H) 3.22 (t, J=6.1 Hz, 2H) 4.69 (m, 2H) 7.41-7.50 (m, 3H) 7.52-7.58 (m, 1H) 7.59 (d, J=0.4 Hz, 1H) 7.76 (br s, 1H) 8.30 (s, exchangeable with D₂₀, 1H) 10.93 (s, exchangeable with D₂₀, 1H);); FABMS found [M]+: 412.1063, 414.1038. C₂₁H₁₉ClN₃O₄ requires 412.1064, 414.1034.

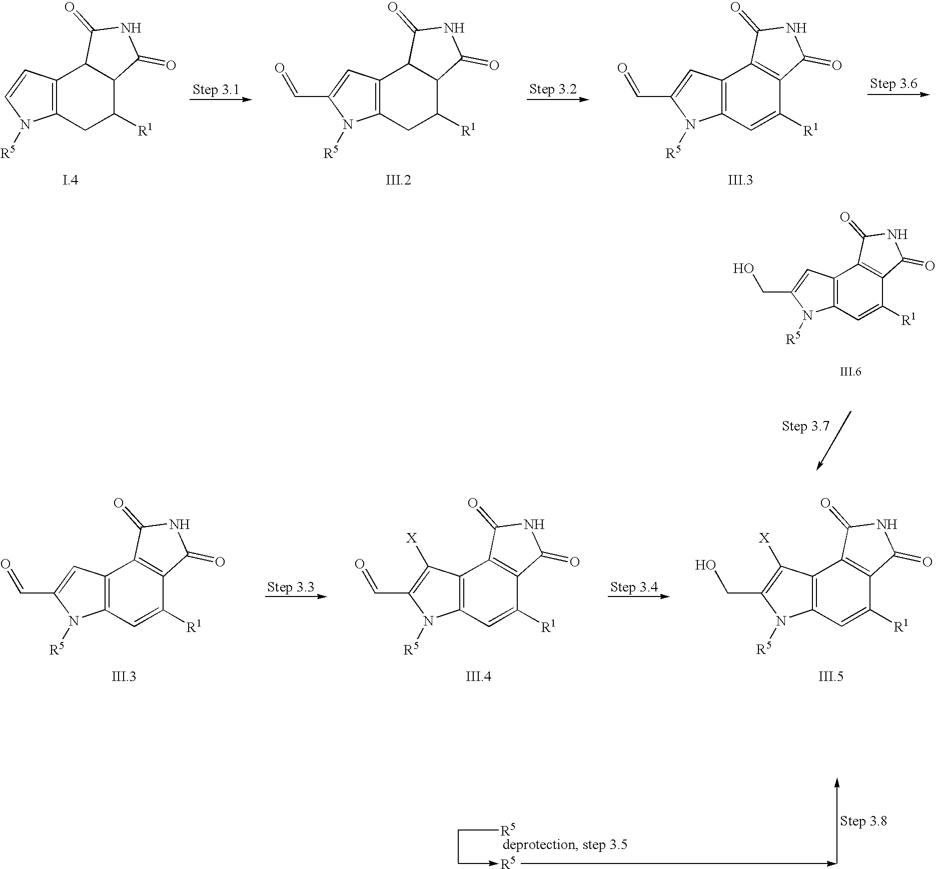

### Procedures for Scheme 3

### EXAMPLE 52

### 4-(2-Chlorophenyl)-6-(2-methoxyethyl)-1,3-dioxo-1,2,3,3a,4,5,6,8b-octahydro-2,6-diaza-as-indacene-7-carbaldehyde

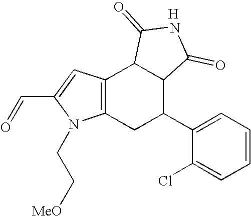

To a solution of 4-(2-chlorophenyl)-6-(2-methoxyethyl)-4,5,6,8b-tetrahydro-3aH-2,6-diaza-as-indacene-1,3-dione (0.75 g), prepared as described in Example 12, in DMF (15 mL) at −30° C., under an inert atmosphere of argon was added POCl₃ (0.3 mL) in small portions. The reaction mixture was stirred cold for 3 hours. 1M NaOH (10 mL) was added followed by water (10 mL). The solid formed was collected and washed with water (20 mL). Azeotopic removal of the residual water with toluene (50 mL) and drying under vacuum 50° C. gave the title compound (0.7 g), ¹H NMR (400 MHz, DMSO-D6) δ ppm 3.02 (m, 2H) 3.11 (s, 3H) 3.48 (m, 3H) 3.83 (dd, J=8.30, 4.15 Hz, 1H) 4.03 (d, J=8.06 Hz, 1H) 4.37 (m, 2H), 7.03 (s, 1H), 7.27 (td, J=7.57, 1.71 Hz, 1H) 7.33 (td, J=7.57, 1.47 Hz, 1H) 7.42 (dd, J=7.82, 1.47 Hz, 1H) 7.54 (dd, J=7.81, 1.47 Hz, 1H) 9.42 (d, J=4.64 Hz, 1H) 10.96 (s, 1H), Mass Spectrum (APCI+) MH+ 387, 389.

### EXAMPLE 53

### 4-(2-Chlorophenyl)-6-(2-methoxyethyl)-1,3-dioxo-1,2,3,6-tetrahydro-2,6-diaza-as-indacene-7-carbaldehyde

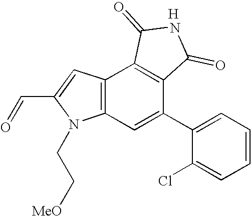

MnO₂ (2 g) and benzene (50 mL) were heated to reflux under a Dean-Stark trap containing 4 A sieves to remove water After 1 hour the reaction mixture was cooled and 4-(2-chlorophenyl)-6-(2-methoxyethyl)-1,3-dioxo-1,2,3,3a,4,5,6,8b-octahydro-2,6-diaza-as-indacene-7-carbaldehyde (0.25 g) prepared as described in Example 51 was added in dioxane (10 mL) with a dioxane wash (5 mL). The reaction mixture was heated to reflux again using the Dean-Stark trap. After 2 hours, dioxane (30 mL) was added and the benzene removed by distillation. The reaction mixture was heated at reflux overnight. After cooling to room temperature, the reaction mixture was filtered through Celite and concentrated to dryness. The mixture of products isolated in dioxane (60 mL) was heated at reflux with MnO₂ (2 g) for 6 hrs. After cooling to room temperature, the reaction mixture was filtered through Celite and concentrated to dryness. The mixture of products isolated was heated at reflux with MnO₂ (2 g) in dioxane (60 mL) for 6 hrs. Once cooled to room temperature, the reaction mixture was filtered through Celite and concentrated to dryness. Purification by sinter flash column chromatography on silica using a gradient of 5% to 50% ethyl acetate in hexane gave the title compound (0.122 g); ¹H NMR (400 MHz, DMSO-D6) δ ppm 3.13 (s, 3H) 3.61 (t, J=5.06 Hz, 2H) 4.82 (t, J=4.98 Hz, 2H) 7.46 (m, 4H) 7.56 (m, 1H) 7.82 (d, J=0.73 Hz, 1H) 7.94 (d, J=0.73 Hz, 1H), 10.08 (s 1H), MS (MH−) 384,383,382,381.

### EXAMPLE 54

### 4-(2-Chlorophenyl)-6-[2-(2-ethoxyethoxy)ethyl]-1,3-dioxo-1,2,3,3a,4,5,6,8b-octahydro-2,6-diaza-as-indacene-7-carbaldehyde

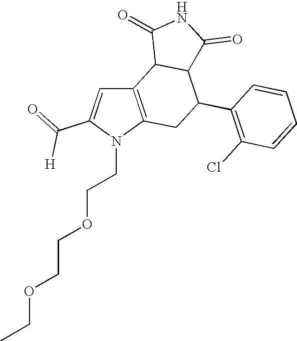

POCl₃ (0.13 mL, 1.44 mmol) was added dropwise under nitrogen at −25° C. to a stirred solution of 4-(2-chlorophenyl)-6-[2-(2-ethoxyethoxy)ethyl]-4,5,6,8b-tetrahydro-3aH-2,6-diaza-as-indacene-1,3-dione (0.50 g, 1.20 mmol), prepared as described in Example 9 in dimethylformamide (8 mL) and stirring was continued at this temperature for 90 min. A solution of NaOH (0.14 g, 3.53 mmol) in water (5 mL) was added the mixture was stirred at room temperature for 30 min. Excess water was added to precipitate out the product, which was washed well with water and air-dried to give the title compound as a tan powder (0.46 g, 87%), mp 190-193° C. API-CI Found: 445, 447 (M+1).

### EXAMPLE 55

### 4-(2-Chlorophenyl)-6-[2-(2-ethoxyethoxy)ethyl]-1,3-dioxo-1,2,3,6-tetrahydro-2,6-diaza-as-indacene-7-carbaldehyde

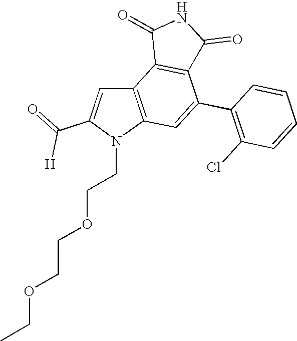

Aromatisation of 4-(2-chlorophenyl)-6-[2-(2-ethoxyethoxy)ethyl]-1,3-dioxo-1,2,3,3a,4,5,6,8b-octahydro-2,6-diaza-as-indacene-7-carbaldehyde, prepared as described in Example 54 with MnO₂ as described in Example 29 gave the title compound (18%) as a tan powder, mp 162-165° C. ¹H NMR (400 MHz, DMSO-D6) δ ppm 0.91 (t, J=7.0 Hz, 3H), 3.19 (q, J=7.0 Hz, 2H), 3.25-3.30 (m), 3.37-3.40 (m, 2H), 3.71 (t, J=5.0 Hz, 2H), 4.82 (t, J=5.0 Hz, 2H), 7.42-7.50 (m, 3H), 7.54-7.58 (m, 1H), 7.82 (d, J=0.7 Hz, 1H), 7.93 (d, J=0.7 Hz, 1H), 10.10 (s, 1H), 11.07 (br s, 1H). API-CI Found: 439, 441 (M−1).

### EXAMPLE 56

### 8-Bromo-4-(2-chlorophenyl)-6-[2-(2-ethoxyethoxy)ethyl]-1,3-dioxo-1,2,3,6-tetrahydro-2,6-diaza-as-indacene-7-carbaldehyde

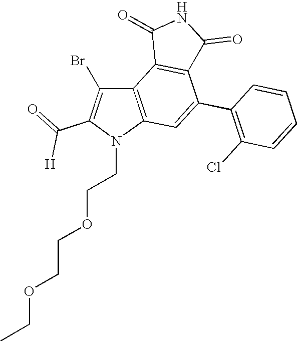

Bromination of 4-(2-chlorophenyl)-6-[2-(2-ethoxyethoxy)ethyl]-1,3-dioxo-1,2,3,6-tetrahydro-2,6-diaza-as-indacene-7-carbaldehyde as described in Example 28 gave the titile compound (86%) which crystallised from ethyl acetate/petroleum ether as a yellow powder, mp 203-205° C. ¹H NMR (400 MHz, DMSO-D6) δ ppm 0.91 (t, J=7.0 Hz, 3H), 3.19 (q, J=7.0 Hz, 2H), 3.22-3.41 (m), 3.70 (t, J=5.0 Hz, 2H), 4.83 (t, J=4.9 Hz, 2H), 7.42-7.50 (m, 3H), 7.54-7.58 (m, 1H), 8.01 (s, 1H), 10.18 (s, 1H), 11.14 (br s, 1H). API-CI Found: 517, 519, 520 (M−1). Elemental Analysis for C₂₃H₂₀BrClN₂O₅, % C (calc/found) 53.15/53.23, % H 3.88/4.16, % N 5.39/5.28.

### EXAMPLE 57

### 4-(2-Chlorophenyl)-6-(3-methoxypropyl)-1,3-dioxo-1,2,3,3a,4,5,6,8b-octahydro-2,6-diaza-as-indacene-7-carbaldehyde

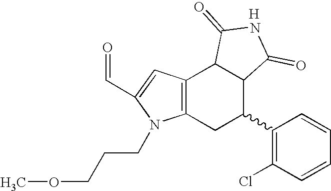

To a stirred solution of 4-(2-chlorophenyl)-6-(3-methoxypropyl)-4,5,6,8b-tetrahydro-3aH-2,6-diaza-as-indacene-1,3-dione (6.5 g, 17.5 mmol), prepared as described in Example 16 in dry DMF (63 mL) was added POCl₃ (2.00 mL, 21.2 mmol) dropwise at −20 to −30° C. (ethylene glycol/dry ice bath). The reaction mixture was further stirred at this temperature for 2 h. 1N NaOH (63 mL) was added slowly, followed by water (100 mL). The resulting mixture was stirred at 0° C. for 1 h. The orange precipitate was filtered off, washed with cold water and dried to give the title compound (a mixture of isomers) (6.6 g, 94%); API-CI Found: 401.0 (M+1, 100%), 403.0 (33); 399.0 (M−1, 100%), 401.0 (30); ¹H NMR (400 MHz, DMSO-D6) δ ppm 1.80-1.90 (m, 2H) 2.95-4.34 (m, 12H) 7.05-7.59 (m, 5H) 9.45 & 9.47 (2s, 1H) 8.01 (s, 1H) 10.96 & 11.29 (2 br s, 1H).

### EXAMPLE 58

### 4-(2-Chlorophenyl)-6-(3-methoxypropyl)-1,3-dioxo-1,2,3,6-tetrahydro-2,6-diaza-as-indacene-7-carbaldehyde

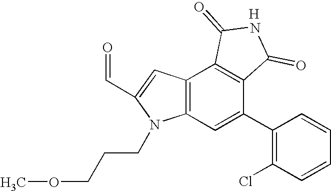

4-(2-chlorophenyl)-6-(3-methoxypropyl)-1,3-dioxo-1,2,3,3a,4,5,6,8b-octahydro-2,6-diaza-as-indacene-7-carbaldehyde (6.6 g, 16.5 mmol) was treated with MnO₂ (3×15 g) in p-dioxane (400 mL) at reflux for 9 h. The mixture was cooled, filtered and the residue washed with ethyl acetate/methanol=10:1. The combined filtrates was evaporated to give crude product which was purified by a silica column (eluting with dichloromethane) to give the title compound (1.61 g, 25%); mp 155-156° C.; API-CI Found: 397.0 (M+1, 100%), 399.0 (33); 395.0 (M−1, 100%), 397.0 (35); ¹H NMR (400 MHz, DMSO-D6) δ ppm 1.95 (m, 2H) 3.10 (s, 3H) 3.23 (t, J=6.0 Hz, 2H) 4.67 (t, J=6.8 Hz, 2H) 7.43-7.52 (m, 3H) 7.55-7.59 (m, 1H) 7.85 (d, J=0.6 Hz, 1H) 7.88 (br s, 1H) 10.09 (s, 1H) 11.08 (s, 1H); FABMS found [M+H]⁺: 397.0950, 399.0937. C₂₁H₁₈ClN₂O₄ requires 397.0955, 399.0925.

### EXAMPLE 59

### 4-(2-Chlorophenyl)-7-hydroxymethyl-6-(3-methoxypropyl)-6H-2,6-diaza-as-indacene-1,3-dione

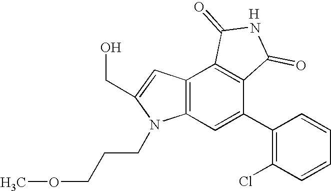

To a stirred solution of 4-(2-chlorophenyl)-6-(3-methoxypropyl)-1,3-dioxo-1,2,3,6-tetrahydro-2,6-diaza-as-indacene-7-carbaldehyde (120 mg, 0.303 mmol), prepared as described in Example 58 in THF (2 mL) at 0° C. under a nitrogen atmosphere, was added BH₃.SMe₂ (2M in THF) (0.08 mL, 0.161 mmol) and the reaction mixture was further stirred at 0° for 1 h. It was quenched with ice and partitioned between ethyl acetate and water. The ethyl acetate solution was dried (Na₂SO₄) and evaporated to dryness. The crude product was purified by a silica column (chloroform/methanol=20:1) to give the title compound (68 mg, 56%); mp 187-189° C.; API-CI Found: 397.0 (M−1, 100%), 399.0 (33); ¹H NMR (400 MHz, DMSO-D6) δ ppm 1.95 (m, 2H) 3.17 (s, 3H) 3.22 (t, J=5.9 Hz, 2H) 4.33 (t, J=7.2 Hz, 2H) 4.74 (d, J=3.2 Hz, 2H) 5.48 (t, J=3.2 Hz, exchangeable with D₂₀, 1H) 7.39-7.48 (m, 3H) 7.51-7.56 (m, 1H) 6.83 (s, 1H) 8.30 (s, 1H) 10.80 (s, exchangeable with D₂₀, 1H); FABMS found [M+H]⁺: 399.1113, 401.1091. C₂₁H₂₀ClN₂O₄ requires 399.1111, 401.1082.

### EXAMPLE 60

### 8-Bromo-4-(2-chlorophenyl)-7-hydroxymethyl-6-(3-methoxypropyl)-6H-2,6-diaza-as-indacene-1,3-dione

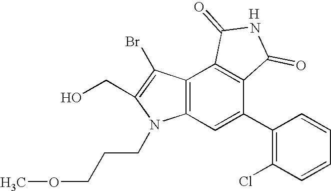

Using the method described in Example 44, 4-(2-chlorophenyl)-7-hydroxymethyl-6-(3-methoxypropyl)-6H-2,6-diaza-as-indacene-1,3-dione (46 mg, 0.116 mmol), prepared as described in Example 59 was treated with bromine (32 mg, 0.202 mmol) in a mixed solvent of chloroform (3 mL) and THF (2 mL) for 20 min to give the title compound (55 mg, 100%) as an orange solid; mp 178-180° C.; API-CI Found: 458.8 (M−17, 80%), 460.8 (100), 462.8 (25); ¹H NMR (400 MHz, DMSO-D6) δ ppm 1.99 (m, 2H) 3.16 (s, 3H) 3.28 (t, J=5.9 Hz, 2H) 4.45 (t, J=7.4 Hz, 2H) 4.78 (d, J=4.3 Hz, 2H) 5.53 (t, J=4.3 Hz, 1H) 7.40-7.49 (m, 3H) 7.52-7.57 (m, 1H) 7.77 (s, 1H) 10.92 (s, 1H); FABMS found [M+H]⁺: 477.0205, 479.0196, 481.0175. C₂₁H₁₉BrClN₂O₄ requires 477.0216, 479.0196 (479.0187), 481.0166.

### EXAMPLE 61

### 8-Bromo-4-(2-chlorophenyl)-6-(3-methoxypropyl)-1,3-dioxo-1,2,3,6-tetrahydro-2,6-diaza-as-indacene-7-carbaldehyde

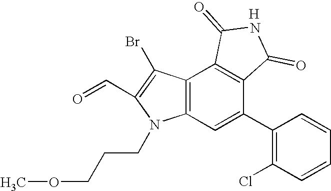

Using the method described in Example 44, 4-(2-chlorophenyl)-6-(3-methoxypropyl)-1,3-dioxo-1,2,3,6-tetrahydro-2,6-diaza-as-indacene-7-carbaldehyde (0.59 g, 1.49 mmol) was treated with bromine (0.41 g, 2.53 mmol) to give the title compound (0.71 g, 100%) as an orange solid; mp 206-207° C.; API-CI Found: 474.8 (M+1, 70%), 476.8 (100), 478.8 (30); ¹H NMR (400 MHz, DMSO-D6) □ ppm 1.93 (m, 2H) 3.09 (s, 3H) 3.23 (t, J=6.1 Hz, 2H) 4.67 (t, J=6.8 Hz, 2H) 7.43-7.51 (m, 3H) 7.54-7.59 (m, 1H) 7.97 (s, 1H) 10.18 (s, 1H) 11.15 (s, 1H); FABMS found [M+H]⁺: 475.0065, 477.0045, 479.0018. C₂₁H₁₇BrClN₂O₄ requires 475.0060, 477.0039 (477.0030), 479.0010.

### EXAMPLE 62

### 8-Bromo-6-(3-bromopropyl)-4-(2-chlorophenyl)-1,3-dioxo-1,2,3,6-tetrahydro-2,6-diaza-as-indacene-7-carbaldehyde

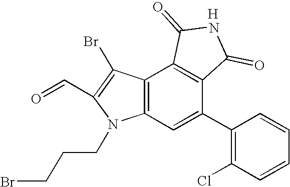 and

### 8-Bromo-4-(2-chlorophenyl)-6-(3-hydroxypropyl)-1,3-dioxo-1,2,3,6-tetrahydro-2,6-diaza-as-indacene-7-carbaldehyde

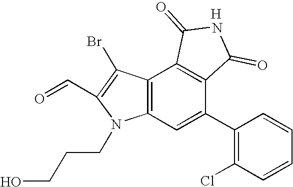

A solution of 8-bromo-4-(2-chlorophenyl)-6-(3-methoxypropyl)-1,3-dioxo-1,2,3,6-tetrahydro-2,6-diaza-as-indacene-7-carbaldehyde (0.65 g, 1.37 mmol) in dichloromethane (30 mL) was treated with BBr₃ (1M in dichloromethane) (4 mL) at 20° C. for 2 h. The solution was quenched with ice and partitioned between ethyl acetate and water. The organic layer was separated, washed again with water, dried (Na₂SO₄), evaporated and the residue chromatographed on a silica column (dichloromethane/ethyl acetate=20:1) into (i) 8-bromo-6-(3-bromopropyl)-4-(2-chlorophenyl)-1,3-dioxo-1,2,3,6-tetrahydro-2,6-diaza-as-indacene-7-carbaldehyde (72 mg, 10%) as an orange solid; mp 229-230° C.; ¹H NMR (400 MHz, DMSO-D6) δ ppm 2.24 (m, 2H) 3.53 (m, 2H) 4.73 (t, J=7.2 Hz, 2H) 7.43-7.52 (m, 3H) 7.54-7.59 (m, 1H) 8.10 (s, 1H) 10.18 (s, 1H) 11.16 (s, 1H); FABMS found [M+H]⁺: 522.9052, 524.9036, 526.9024, 528.8981. C₂øH₁₄Br₂ClN₂O₃ requires 522.9059, 524.9039 (524.9030), 526.9018 (526.9009), 528.8989; and (ii) 8-bromo-4-(2-chlorophenyl)-6-(3-hydroxypropyl)-1,3-dioxo-1,2,3,6-tetrahydro-2,6-diaza-as-indacene-7-carbaldehyde (67 mg, 11%); API-CI Found: 460.8 (M+1, 75%), 462.80 (100), 464.8 (30); The ¹H NMR spectrum showed the presence of minor impurities which were unable to be removed by further chromatography or by re-crystallisation. This impure sample was reduced directly to give 8-bromo-4-(2-chlorophenyl)-7-hydroxymethyl-6-(3-hydroxypropyl)-6H-2,6-diaza-as-indacene-1,3-dione (Example 63).

### EXAMPLE 63

### 8-Bromo-4-(2-chlorophenyl)-7-hydroxymethyl-6-(3-hydroxypropyl)-6H-2,6-diaza-as-indacene-1,3-dione

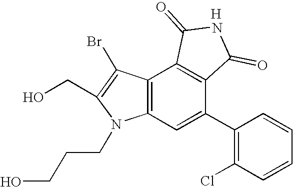

The impure sample of 8-bromo-4-(2-chlorophenyl)-6-(3-hydroxypropyl)-1,3-dioxo-1,2,3,6-tetrahydro-2,6-diaza-as-indacene-7-carbaldehyde (67 mg, 0.146 mmol), prepared as described in Example 62, in THF (5 mL) was reduced with BH₃.SMe₂ (2M in THF) (0.05 mL) at 0° C. for 40 min. The solution was partitioned between ethyl acetate and water. The organic layer was washed with water, dried (Na₂SO₄), and evaporated to give an orange solid which was recrystallised from ethyl acetate/petroleum ether to afford the title compound (46 mg, 68%); mp 203-205° C.; API-CI Found: 444.8 (M−17, 90%), 446.8 (100), 448.8 (20); ¹H NMR (400 MHz, DMSO-D6) δ ppm 1.90 (m, 2H) 3.41 (m, 2H) 4.47 (t, J=7.5 Hz, 2H) 4.63 (t, J=5.0 Hz, 1H) 4.80 (d, J=5.2 Hz, 2H) 5.53 (t, J=5.2 Hz, 1H) 7.40-7.49 (m, 3H) 7.52-7.57 (m, 1H) 7.80 (s, 1H) 10.91 (s, 1H); FABMS found [M]+: 463.0046, 465.0046, 467.0017. C₂₀H₁₇BrClN₂O₄ requires 463.0060, 465.0039 (465.0030), 467.0010.

### EXAMPLE 64

### 8-Chloro-4-(2-chlorophenyl)-6-(3-methoxypropyl)-1,3-dioxo-1,2,3,6-tetrahydro-2;6-diaza-as-indacene-7-carbaldehyde

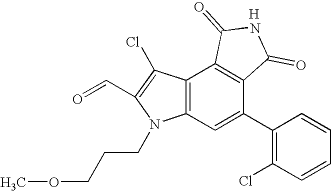

A solution of N-chlorosuccinimide (NCS) (74 mg, 0.55 mmol) in DMF (2 mL) was added dropwise at 0° C. under a nitrogen atmosphere to a stirred solution of 4-(2-chlorophenyl)-6-(3-methoxypropyl)-1,3-dioxo-1,2,3,6-tetrahydro-2,6-diaza-as-indacene-7-carbaldehyde (198 mg, 0.50 mmol), prepared as described in Example 58 in DMF (5 mL). The reaction was stirred at 0° for 40 min and then at 20° overnight (13 h). It was partitioned between ethyl acetate and water. The organic layer was separated, washed with water, dried (Na₂SO₄) and evaporated to give an oil which was purified by a silica column (ethyl acetate/petroleum ether=1:3) to afford the title compound (215 mg, 100%); mp 198-199° C.; API-CI Found: 430.8 (M+1, 100%), 432.8 (65); 428.8 (M−1, 100%), 430.8 (65); ¹H NMR (400 MHz, DMSO-D6) δ ppm 1.93 (m, 2H) 3.09 (s, 3H) 3.23 (t, J=6.0 Hz, 2H) 4.66 (t, J=6.8 Hz, 2H) 7.43-7.52 (m, 3H) 7.54-7.59 (m, 1H) 7.96 (s, 1H) 10.22 (s, 1H) 11.17 (s, 1H); FABMS found [M+H]⁺: 431.0557, 433.0535, 435.0526. C₂₁H₁₇Cl₂N₂O₄ requires 431.0565, 433.0535, 435.0506.

### EXAMPLE 65

### 8-Chloro-6-(3-bromopropyl)-4-(2-chlorophenyl)-1,3-dioxo-1,2,3,6-tetrahydro-2,6-diaza-as-indacene-7-carbaldehyde

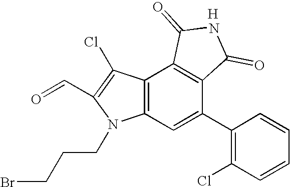 and

### 8-Chloro-4-(2-chlorophenyl)-6-(3-hydroxypropyl)-1,3-dioxo-1,2,3,6-tetrahydro-2,6-diaza-as-indacene-7-carbaldehyde

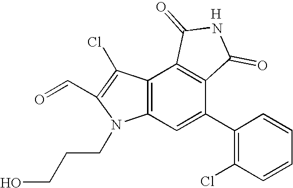

4-(2-Chlorophenyl)-6-(3-methoxypropyl)-1,3-dioxo-1,2,3,6-tetrahydro-2,6-diaza-as-indacene-7-carbaldehyde (104 mg, 0.242 mmol), prepared as described in Example 64 in dichloromethane (10 mL) was treated with BBr₃ (1M in dichloromethane) (1.5 mL) at 0° C. for 3 h and then at 5° for 16 h. It was quenched with ice and partitioned between dichloromethane and water. The organic layer was separated washed again with water; dried (Na₂SO₄), evaporated and chromatographed on a silica column (petroleum ether/ethyl acetate=2:1) into (i) 8-chloro-6-(3-bromopropyl)-4-(2-chlorophenyl)-1,3-dioxo-1,2,3,6-tetrahydro-2,6-diaza-as-indacene-7-carbaldehyde (18 mg, 15%) as an orange solid; mp 225-226° C.; API-CI Found: 478.8 (M+1, 50%), 480.8 (80), 482.8 (45); ¹H NMR (400 MHz, DMSO-D6) δ ppm 2.25 (m, 2H) 3.52 (m, 2H) 4.72 (t, J=7.2 Hz, 2H) 7.43-7.52 (m, 3H) 7.55-7.59 (m, 1H) 8.09 (s, 1H) 10.22 (s, 1H) 11.19 (s, 1H); FABMS found [M+H]⁺: 478.9548, 480.9536, 482.9513. C₂₀H₁₄BrCl₂N₂O₃ requires 478.9564, 480.9535 (480.9544), 482.9514 (482.9505); and (ii) 8-chloro-4-(2-chlorophenyl)-6-(3-hydroxypropyl)-1,3-dioxo-1,2,3,6-tetrahydro-2,6-diaza-as-indacene-7-carbaldehyde (80 mg, 79%); API-CI Found: 416.8 (M+1, 85%), 418.8 (50); 414.8 (M−1, 100%), 415.8 (40), 416.8 (75), 417.8 (25). The ¹H NMR spectrum showed the presence of minor impurities which were unable to be removed by further chromatography or by recrystallisation. This impure sample was reduced directly to give 8-chloro-4-(2-chlorophenyl)-7-hydroxymethyl-6-(3-hydroxypropyl)-6H-2,6-diaza-as-indacene-1,3-dione.

### EXAMPLE 66

### 8-Chloro-4-(2-chlorophenyl)-7-hydroxymethyl-6-(3-hydroxypropyl)-6H-2,6-diaza-as-indacene-1,3-dione

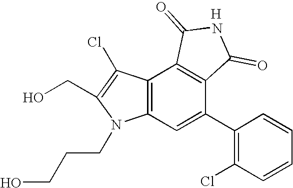

The impure sample of 8-chloro-4-(2-chlorophenyl)-6-(3-hydroxypropyl)-1,3-dioxo-1,2,3,6-tetrahydro-2,6-diaza-as-indacene-7-carbaldehyde (72 mg, 0.173 mmol), prepared as described in Example 65 in THF (2 mL) was reduced with BH₃.SMe₂ (2M in THF) (0.044 mL, 0.087 mmol) at 0° C. for 1 h 45 min. It was quenched with ice at 0° C. and partitioned between ethyl acetate and water. The organic solution was further washed with water, dried (Na₂SO₄), and evaporated to give a yellow oil which was chromatographed on a silica column (ethyl acetate/dichloromethane=1:5) to afford the title compound (46 mg, 64%); mp 218-219° C.; API-CI Found: 415.8 (M−1, 100%), 416.8 (80), 418.8 (40); ¹H NMR (400 MHz, DMSO-D6) δ ppm 1.90 (m, 2H) 3.40 (m, 2H) 4.44 (t, J=7.4 Hz, 2H) 4.63 (t, J=4.9 Hz, exchangeable with D₂₀, 1H) 4.77 (br s, 2H) 5.54 (br s, exchangeable with D₂₀, 1H) 7.40-7.49 (m, 3H) 7.52-7.57 (m, 1H) 7.79 (s, 1H) 10.92 (s, exchangeable with D₂₀, 1H); FABMS found [M+H]⁺: 419.0546, 421.0533, 423.0516. C₂₀H₁₇Cl₂N₂O₄ requires 419.0565, 421.0535, 423.0506.

### EXAMPLE 67

### 8-Chloro-4-(2-chlorophenyl)-7-hydroxymethyl-6-(3-methoxypropyl)-6H-2,6-diaza-as-indacene-1,3-dione

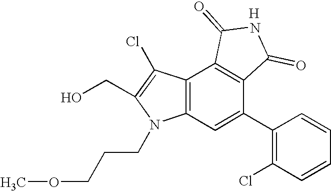

A solution of 8-chloro-4-(2-chlorophenyl)-6-(3-methoxypropyl)-1,3-dioxo-1,2,3,6-tetrahydro-2,6-diaza-as-indacene-7-carbaldehyde (74 mg, 0.172 mmol), prepared as described in Example 64, in dichloromethane (10 mL) was reduced with BH₃.SMe₂ (2M in THF) (0.5 mL, 1.00 mmol) at 0° C. for 3 h and worked up as described in Example 66 to give the title compound (75 mg, 100%) as a yellow solid; mp 214-215° C.; API-CI Found: 415.0 (M−17, 100%), 416.8 (70); ¹H NMR (400 MHz, DMSO-D6) δ ppm 1.90 (m, 2H) 3.16 (s, 3H) 3.28 (t, J=5.9 Hz, 2H) 4.43 (t, J=7.3 Hz, 2H) 4.76 (br s, 2H) 5.54 (br s, 1H) 7.40-7.49 (m, 3H) 7.52-7.57 (m, 1H) 7.76 (s, 1H) 10.90 (s, 1H); Elemental Analysis for C₂₁H₁₈Cl₂N₂O₄, % C (calc/found) 58.21/58.31, % H 4.19/4.30, % N 6.47/6.66.

### EXAMPLE 68

### 4-(2-Chlorophenyl)-6-[3-(2-methoxyethoxy)propyl]-1,3-dioxo-1,2,3,6-tetrahydro-2,6-diaza-as-indacene-7-carbaldehyde

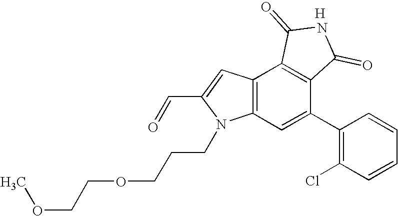

To a stirred solution of 4-(2-chlorophenyl)-6-(3-methoxypropyl)-4,5,6,8b-tetrahydro-3aH-2,6-diaza-as-indacene-1,3-dione (0.96 g, 2.31 mmol), prepared as described in Example 17 in dry DMF (8.3 mL) was added POCl₃ (0.263 mL, 2.79 mmol) dropwise at −20 to −30° C. (ethylene glycol/dry ice bath). The reaction mixture was stirred at this temperature for 1 h 15 min. 1N NaOH (8.3 mL) was added slowly and stirring was continued at this temperature for 30 min, then at room temperature overnight. The mixture was partitioned between ethyl acetate and water and the organic layer was separated and washed again with water, dried (Na₂SO₄) and evaporated to give an orange oil (1.07 g) which was treated with MnO₂ (3×6 g) in p-dioxane (100 mL) at reflux for 7 h. The reaction mixture was filtered through Celite and the residue washed with ethyl acetate. The combined filtrates were evaporated to give the title compound (0.19 g, 19%) as an orange solid; API-CL Found: 441.0 (M+1, 100%), 443.0 (70); 439.0 (M−1, 100%), 441.0 (33); ¹H NMR (400 MHz, DMSO-D6) δ ppm 1.95 (m, 2H) 3.09 (s, 3H) 3.22-3.37 (m, 6H) 4.67 (t, J=6.7 Hz, 2H) 7.42-7.51 (m, 3H) 7.54-7.59 (m, 1H) 7.85 (d, J=0.4 Hz, 1H) 7.89 (br s, 1H) 10.09 (s, 1H) 11.07 (s, 1H); FABMS found [M+H]⁺: 441.1223, 443.1194. C₂₃H₂₂ClN₂O₅ requires 441.1217, 443.1187.

### EXAMPLE 69

### 8-Bromo-4-(2-chlorophenyl)-6-[3-(2-methoxyethoxy)propyl]-1,3-dioxo-1,2,3,6-tetrahydro-2,6-diaza-as-indacene-7-carbaldehyde

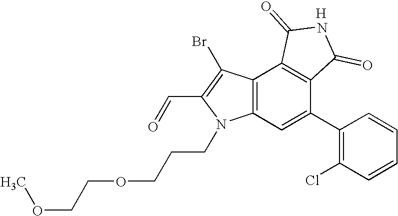

Using the method described in Example 44, 4-(2-Chlorophenyl)-6-[3-(2-methoxyethoxy)propyl]-1,3-dioxo-1,2,3,6-tetrahydro-2,6-diaza-as-indacene-7-carbaldehyde (66 mg, 0.15 mmol), prepared as described in Example 68 was treated with bromine (65 mg, 0.61 mmol) to give the title compound (72 mg, 92%) as an orange solid; mp 168-169° C.; API-CI Found: 518.8 (M+1, 70%), 520.8 (100), 522.8 (20); 516.8 (M−1, 20%), 518.8 (30%), 519.9 (8%); ¹H NMR (400 MHz, DMSO-D6) □ ppm 1.93 (m, 2H) 3.09 (s, 3H) 3.20 (t, J=4.8 Hz, 2H) 3.24-3.40 (m, 4H) 4.67 (t, J=6.6 Hz, 2H) 7.42-7.52 (m, 3H) 7.54-7.58 (m, 1H) 7.97 (s, 1H) 10.18 (s, 1H) 11.20 (s, 1H); FABMS found [M+H]⁺: 519.0316, 521.0304, 523.0276. C₂₃H₂₁BrClN₂O₅ requires 519.0322, 521.0301 (521.0292), 523.0272.

### EXAMPLE 70

### 8-Bromo-4-(2-chlorophenyl)-7-hydroxymethyl-6-[[3-(2-methoxyethoxy)propyl]-6H-2,6-diaza-as-indacene-1,3-dione

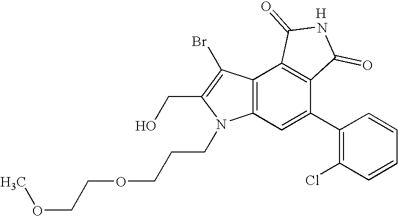

8-Bromo-4-(2-chlorophenyl)-6-[3-(2-methoxyethoxy)propyl]-1,3-dioxo-1,2,3,6-tetrahydro-2,6-diaza-as-indacene-7-carbaldehyde (56 mg, 0.108 mmol), prepared as described in Example 69, in dichloromethane (8 mL) was reduced with BH₃.SMe₂ (2M in THF) (0.3 mL, 0.60 mmol) at 0° C. for 2 h and worked up using the procedures described in Example 66. Chromatography of the crude product on a silica column (chloroform/methanol=10:1) gave the title compound (56 mg, 99%) as a reddish brown solid; mp 149-150° C.; API-CI Found: 502.8 (M−17, 85%), 504.8 (100) 506.8 (30); ¹H NMR (400 MHz, DMSO-D6) δ ppm 1.99 (m, 2H) 3.14 (s, 3H) 3.26-3.42 (m, 6H) 4.46 (t, J=7.0 Hz, 2H) 4.79 (d, J=5.3 Hz, 2H) 5.52 (t, J=5.3 Hz, 1H) 7.40-7.49 (m, 3H) 7.52-7.57 (m, 1H) 7.78 (s, 1H) 10.90 (s, 1H); FABMS found [M]⁺: 520.0399, 522.0381, 524.0379. C₂₃H₂₂BrClN₂O₅ requires 520.0400, 522.0380 (522.0371), 524.0350.

### EXAMPLE 71

### 4-(2-Chlorophenyl)-6-methyl-1,3-dioxo-1,2,3,3a,4,5,6,8b-octahydro-2,6-diaza-as-indacene-7-carbaldehyde

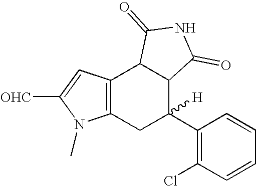

To a solution of 4-(2-chlorophenyl)-6-methyl-4,5,6,8b-tetrahydro-3aH-2,6-diaza-as-indacene-1,3-dione (375 g, 1.2 mol), prepared as described in Example 3, in DMF (2500 mL), under nitrogen at −30° C., was slowly added phosphorus oxychloride (298 g, 1.95 mol). The reaction mixture was stirred for 2 h at −30° C. Sodium hydroxide (1M, 1000 mL) was added slowly. The reaction mixture was allowed to warm to room temperature and stirred overnight. The brown solid was collected by filtration, washed with water and dried under high vacuum at 40° C. to afford 349 g (85%) of title compound (mixture of isomers) as a brown solid; LCMS (20 mm C18-MAX-RP column, gradient method 2-95% acetonitrile in water with (0.1% formic acid) in 3.3 min with 2 min hold at 95% acetonitrile in water with (0.1% formic acid), MSD in ESI positive mode) 14.9% at 2.70 min, 75.2% at 2.79 min (UV_{210 nm}); API-ES 343.1 (100), 344.0 (20), 345.2 (25), 365.0 (20), 367.1 (10).

### EXAMPLE 72

### 4-(2-Chlorophenyl)-6-methyl-1,3-dioxo-1,2,3,6-tetrahydro-2,6-diaza-as-indacene-7-carbaldehyde

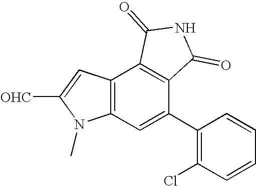

A suspension of 4-(2-chlorophenyl)-6-methyl-1,3-dioxo-1,2,3,3a,4,5,6,8b-octahydro-2,6-diaza-as-indacene-7-carbaldehyde (184 g, 535 mmol), prepared as described in Example 71 and manganese(IV) oxide (<5 micron, 698 g, 8.02 mol) in dioxane (3000 mL) was heated to reflux under nitrogen overnight. Additional manganese(IV) oxide (70 g) was added and the reaction heated to reflux for an additional 9 h. The reaction suspension was cooled to room temperature and diluted with ethyl acetate. The suspension was filtered through a bed of Celite and the filter cake washed with additional ethyl acetate. Concentration of the filtrate, followed by purification of the residue on silica gel (eluent: 5% ethyl acetate in dichloromethane) gave 59.4 g (33%) of the title compound; LCMS (20 mm C18-MAX-RP column, gradient method 2-95% acetonitrile in water with (0.1% formic acid) in 3.3 min with 2 min hold at 95% acetonitrile in water with (0.1% formic acid), MSD in ESI positive mode) 95.13% at 3.55 min (UV_{210 nm}); API-ES 339.0 (100), 339.9 (20), 341.1 (35), 360.9 (15), 363.0 (5); Elemental Analysis for C₁₈H₁₁ClN₂O₃% C (calc/found) 63.82/63.42, % H 3.27/3.04, % N 8.27/7.91, % Cl 10.47/10.85.

### EXAMPLE 73

### 8-Bromo-4-(2-chlorophenyl)-6-methyl-1,3-dioxo-1,2,3,6-tetrahydro-2,6-diaza-as-indacene-7-carbaldehyde

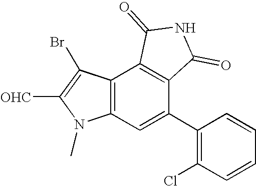

To a solution of 4-(2-chlorophenyl)-6-methyl-1,3-dioxo-1,2,3,6-tetrahydro-2,6-diaza-as-indacene-7-carbaldehyde (2.50 g, 7.38 mmol), prepared as described in Example 72, in THF (25 mL) was added N-bromosuccinimide (3.14 g, 17.6 mmol). The reaction mixture was stirred at reflux for 1 h. The reaction mixture was cooled and hexanes were added (50 mL). The solid that precipitated was filtered and washed with fresh hexanes (25 mL) and water (25 mL). The solid material was dried in a vacuum oven at 60° C. to give 2.75 g of the title compound as a bright yellow solid; ¹H NMR (400 MHz, DMSO-D6) δ ppm 4.09 (s, 3H) 7.47 (m, 3H) 7.55 (m, 1H) 8.02 (s, 1H) 10.14 (s, 1H) 11.19 (s, 1H); Elemental Analysis for C₁₈H₁₀BrClN₂O₃.0.34C₃H₈O₂% C (calc/found) 54.88/54.94, % H 3.33/3.04, % N 6.29/5.89.

### EXAMPLE 74

### 8-Chloro-4-(2-chloro-phenyl)-6-methyl-1,3-dioxo-1,2,3,6-tetrahydro-2,6-diaza-as-indacene-7-carbaldehyde

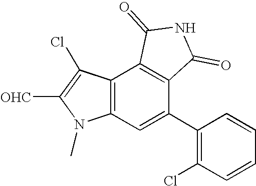

8-Chloro-4-(2-chlorophenyl)-6-methyl-1,3-dioxo-1,2,3,6-tetrahydro-2,6-diaza-as-indacene-7-carbaldehydehyde was prepared from 4-(2-chlorophenyl)-6-methyl-1,3-dioxo-1,2,3,6-tetrahydro-2,6-diaza-as-indacene-7-carbaldehyde (2.50 g, 7.38 mmol) and N-chlorosuccinimide (1.48 g, 11.1 mmol), using the procedure described in Example 73 to give 2.32 g of the title compound as a yellow solid; ¹H NMR (400 MHz, DMSO-D6) 8 ppm 4.09 (s, 3H) 7.48 (m, 3H) 7.56 (m, 1H) 8.02 (s, 1H) 10.20 (s, 1H) 11.21 (s, 1H); Elemental Analysis for C₁₈H₁₀Cl₂N₂O₃.0.75C₃H₈O % C (calc/found) 59.22/59.03, % H 3.39/3.77, % N 6.00/6.56.

### EXAMPLE 75

### 4-(2-Chlorophenyl)-7-hydroxymethyl-6-methyl-6H-2,6-diaza-as-indacene-1,3-dione

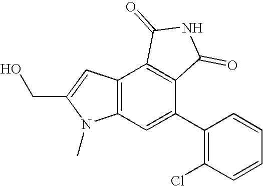

To a solution of 4-(2-chlorophenyl)-6-methyl-1,3-dioxo-1,2,3,6-tetrahydro-2,6-diaza-as-indacene-7-carbaldehyde (0.250 g, 0.74 mmol), prepared as described in Example 72, in ethanol (10 mL) was added sodium borohydride (0.010 g, 0.24 mmol). The reaction mixture was stirred for 15 min and then poured into water (100 mL) and the solid product filtered to give 0.219 g of the title compound as a yellow solid; ¹H NMR (400 MHz, DMSO-D6) δ ppm 3.82 (s, 3H) 4.73 (d, J=5.37 Hz, 2H) 5.48 (t, J=5.49 Hz, 1H) 6.79 (s, 1H) 7.43 (m, 3H) 7.54 (m, 1H) 7.68 (s, 1H) 10.83 (s, 1H); Elemental Analysis for C₁₈H₁₃ClN₂O₃.0.25H₂O, % C (calc/found) 62.62/62.36, % H 3.94/3.66, % N 8.11/8.02.

### EXAMPLE 76

### 8-Bromo-4-(2-chlorophenyl)-7-hydroxymethyl-6-methyl-6H-2,6-diaza-as-indacene-1,3-dione

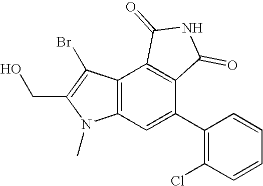

To a solution of 4-(2-chlorophenyl)-7-hydroxymethyl-6-methyl-6H-2,6-diaza-as-indacene-1,3-dione 1.01 (0.150 g, 0.36 mmol), prepared as described in Example 75 in ethanol (10 mL) was added sodium borohydride (0.007 g, 0.18 mmol). The reaction mixture was stirred for 15 min and then poured into water (100 mL) and the solid product filtered to give 0.103 g of the title compound as a yellow solid;

¹H NMR (400 MHz, DMSO-D6) δ ppm 3.91 (s, 3H) 4.76 (d, J=5.37 Hz, 2H) 5.54 (t, J=5.37 Hz, 1H) 7.45 (m, 3H) 7.54 (d, J=7.81 Hz, 1H) 7.81 (s, 1H) 10.95 (s, 1H); Elemental Analysis for C₁₈H₁₂BrClN₂O₃.0.15H₂O, % C (calc/found) 51.19/50.89, % H 2.94/2.33, % N 6.63/6.46.

### EXAMPLE 77

### 8-Chloro-4-(2-chlorophenyl)-7-hydroxymethyl-6-methyl-6H-2,6-diaza-as-indacene-1,3-dione

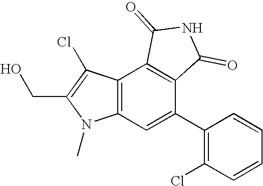

To a solution of 4-(2-chlorophenyl)-7-hydroxymethyl-6-methyl-6H-2,6-diaza-as-indacene-1,3-dione (0.089 g, 0.24 mmol), prepared as described in Example 76 in ethanol (10 mL) was added sodium borohydride (0.005 g, 0.12 mmol). The reaction mixture was stirred for 15 min and then poured into water (100 mL) and the solid product filtered to give 0.022 g of the title compound as a yellow solid; ¹H NMR (400 MHz, DMSO-D6) δ ppm 3.89 (s, 3H) 4.74 (s, 2H) 5.55 (s, 1H) 7.45 (m, 3H) 7.54 (d, J=8.30 Hz, 1H) 7.80 (s, 1H); LCMS (Phenomenex Develosil Combi RP3 50×4.6 mm column, gradient method 70-2% acetonitrile/water eluent (0.1% formic acid) in 2.0 min with 2 min hold, MSD in ESI positive mode) 100% at 2.81 min (UV_{254 nm}); API-ES 277.2 (40), 357.1 (60), 375.1 (100), 377.1 (60).

### EXAMPLE 78

### 6-[2-(tert-Butyldimethylsilanyloxy)ethyl]-4-(2-methoxyphenyl)-1,3-dioxo-1,2,3,3a,4,5,6,8b-octahydro-2,6-diaza-as-indacene-7-carbaldehyde

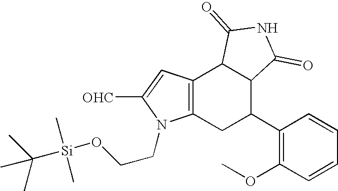

6-[2-(tert-Butyldimethylsilanyloxy)ethyl]-4-(2-methoxyphenyl)-1,3-dioxo-1,2,3,3a,4,5,6,8b-octahydro-2,6-diaza-as-indacene-7-carbaldehyde was prepared from 6-[2-(tert-butyldimethylsilanyloxy)ethyl]4-(2-methoxyphenyl)-4,5,6,8b-tetrahydro-3aH-2,6-diaza-as-indacene-1,3-dione (2.00 g, 4.40 mmol) itself prepared as described in Example 26 and POCl₃ (1.18 g, 7.70 mmol), using the procedure described in Example 52 to give the title compound as a yellow solid which was used without purification; ¹H NMR (400 MHz, DMSO-D6) δ ppm 0.67 (m, 3H) 2.72 (s, 5H) 2.88 (s, 6H) 2.99 (s, 1H) 3.78 (m, 4H) 3.99 (d, J=8.30 Hz, 1H) 4.29 (m, 1H) 6.98 (m, 1H) 7.28 (m, 1H) 7.94 (s, 2H) 9.43 (s, 1H) 10.88 (s, 1H); LCMS (Phenomenex Develosil Combi RP3 50×4.6 mm column, gradient method 25-2% acetonitrile/water eluent (0.1% formic acid) in 2.0 min with 2 min hold, MSD in ESI positive mode) 89% at 1.87 min (UV_{254 nm}); API-ES 483.1 (100), 484.1 (40).

### EXAMPLE 79

### 6-[2-(tert-Butyldimethylsilanyloxy)ethyl]-4-(2-methoxyphenyl)-1,3-dioxo-1,2,3,6-tetrahydro-2,6-diaza-as-indacene-7-carbaldehyde

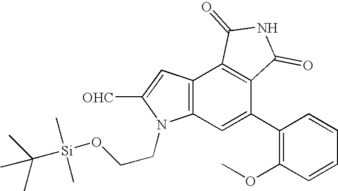

6-[2-(tert-Butyldimethylsilanyloxy)ethyl]4-(2-methoxyphenyl)-1,3-dioxo-1,2,3,6-tetrahydro-2,6-diaza-as-indacene-7-carbaldehyde was prepared from 6-[2-(tert-butyldimethylsilanyloxy)ethyl]4-(2-methoxyphenyl)-1,3-dioxo-1,2,3,3a,4,5,6,8b-octahydro-2,6-diaza-as-indacene-7-carbaldehyde (2.00 g, 4.14 mmol), prepared as described in Example 78 using the procedure described in Example 53 to give 0.605 g of the title compound as a beige solid; ¹H NMR (400 MHz, DMSO-D6) δ ppm −0.30 (m, 6H) 0.60 (m, 9H) 3.64 (s, 3H) 3.87 (t, J=4.76 Hz, 2H) 4.75 (t, J=4.76 Hz, 2H) 7.04 (m, 2H) 7.28 (dd, J=7.57, 1.71 Hz, 1H) 7.41 (m, 1H) 7.82 (d, J=13.19 Hz, 2H) 10.07 (s, 1H) 10.98 (s, 1H); LCMS (Phenomenex Develosil Combi RP3 50×4.6 mm column, gradient method 25-2% acetonitrile/water eluent (0.1% formic acid) in 2.0 min with 2 min hold, MSD in ESI positive mode) 100% at 2.46 min (UV_{254 nm}); API-ES 347.0 (10), 451.0 (15), 479.0 (100), 480.0 (40).

### EXAMPLE 80

### 8-Bromo-6-[2-(tert-butyldimethylsilanyloxy)ethyl]-4-(2-methoxyphenyl)-1,3-dioxo-1,2,3,6-tetrahydro-2,6-diaza-as-indacene-7-carbaldehyde

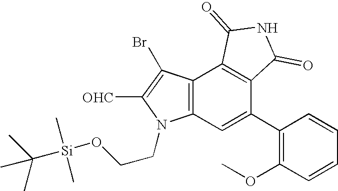

8-Bromo-6-[2-(tert-butyldimethylsilanyloxy)ethyl]-4-(2-methoxyphenyl)-1,3-dioxo-1,2,3,6-tetrahydro-2,6-diaza-as-indacene-7-carbaldehyde was prepared from 6-[2-(tert-butyldimethylsilanyloxy)ethyl]-4-(2-methoxyphenyl)-1,3-dioxo-1,2,3,6-tetrahydro-2,6-diaza-as-indacene-7-carbaldehyde (0.254 g, 0.53 mmol), prepared as described in Example 79 using the procedure described in Example 28 to give 0.295 g of the title compound as a bright yellow solid; ¹H NMR (400 MHz, DMSO-D6) δ ppm −0.30 (m, 5H) 0.58 (m, 8H) 3.63 (s, 3H) 3.85 (t, J=4.88 Hz, 2H) 4.79 (m, 2H) 7.04 (dd, J=15.87, 8.30 Hz, 2H) 7.27 (dd, J=7.33, 1.71 Hz, 1H) 7.41 (m, 1H) 7.92 (s, 1H) 10.17 (s, 1H) 11.07 (s, 1H); LCMS (Phenomenex Develosil Combi RP3 50×4.6 mm column, gradient method 25-2% acetonitrile/water eluent (0.1% formic acid) in 2.0 min with 2 min hold, MSD in ESI positive mode) 87% at 2.93 min (UV_{254 nm}); API-ES 556.9 (80), 558.9 (100), 559.9 (40).

### EXAMPLE 81

### 8-Bromo-6-[2-(tert-butyldimethylsilanyloxy)ethyl]-7-hydroxymethyl-4-(2-methoxyphenyl)-6H-2,6-diaza-as-indacene-1,3-dione

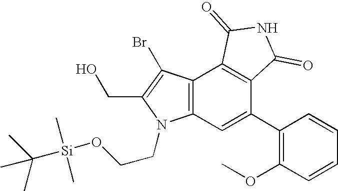

To a solution of 8-bromo-6-[2-(tert-butyldimethylsilanyloxy)ethyl]4-(2-methoxyphenyl)-1,3-dioxo-1,2,3,6-tetrahydro-2,6-diaza-as-indacene-7-carbaldehyde (0.295 g, 0.53 mmol), prepared as described in Example 80 in THF/ethanol (10 mL/2 mL) was added zinc chloride (0.144 g, 1.06 mmol). The reaction mixture was stirred for 10 min then sodium borohydride (0.040 g, 1.06 mmol) was added. The reaction mixture was stirred for 1 h at room temperature and then poured into ethyl acetate (50 mL). The organic phase was washed four times with water (25 mL each), brine (25 mL), dried over MgSO₄ and concentrated. Recrystallization of the crude product from THF/hexanes (1:3) produced 0.216 g of the title compound as a yellow solid; ¹H NMR (400 MHz, DMSO-D6) δ ppm −0.28 (m, 6H) 0.64 (s, 9H) 3.63 (s, 3H) 3.89 (s, 2H) 4.55 (s, 2H) 4.79 (d, J=5.37 Hz, 2H) 5.53 (s, 1H) 7.03 (m, 2H) 7.25 (m, 1H) 7.37 (s, 1H) 7.74 (s, 1H) 10.82 (s, 1H); LCMS (Phenomenex Develosil Combi RP3 50×4.6 mm column, gradient method 25-2% acetonitrile/water eluent (0.1% formic acid) in 2.0 min with 2 min hold, MSD in ESI positive mode) 91% at 1.90 min (UV_{254 nm}); API-ES 540.9 (80), 542.9 (100), 558.9 (50), 560.9 (60).

### EXAMPLE 82

### 8-Bromo-6-(2-hydroxyethyl)-7-hydroxymethyl-4-(2-methoxyphenyl)-6H-2,6-diaza-as-indacene-1,3-dione

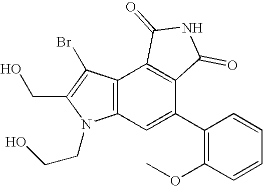

To a solution of 8-bromo-6-[2-(tert-butyldimethylsilanyloxy)ethyl]-7-hydroxymethyl-4-(2-methoxyphenyl)-6H-2,6-diaza-as-indacene-1,3-dione (0.216 g, 0.39 mmol), prepared as described in Example 81 in DMF (5 mL) was added ammonium fluoride (0.357 g, 9.651 mmol). The reaction mixture was stirred for 3 h at 50° C. then at room temperature for 72 h. The reaction mixture was poured into ethyl acetate (50 mL). The organic phase was washed three times with water (25 mL each), brine (25 mL), dried over MgSO₄ and concentrated. Recrystallization of the crude product from THF/hexanes (1:3) produced 0.1115 g of the title compound as a bright yellow solid; ¹H NMR (400 MHz, DMSO-D6) δ ppm 1.75 (m, 1H) 3.59 (m, J=6.56, 4.30, 2.08, 1.95 Hz, 1H) 3.64 (s, 3H) 3.69 (q, J=5.29 Hz, 2H) 4.47 (t, J=5.25 Hz, 2H) 4.79 (d, J=5.37 Hz, 2H) 5.00 (m, 1H) 5.50 (m, 1H) 7.02 (m, 2H) 7.27 (dd, J=7.33, 1.71 Hz, 1H) 7.40 (m, 1H) 7.76 (s, 1H) 10.82 (s, 1H); LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 94% at 1.104 in (UV_{214 nm}); API-ES 347.0 (15), 424.9 (900, 426.9 (100), 427.9 (25), 444.9 (50), 446.9 (50).

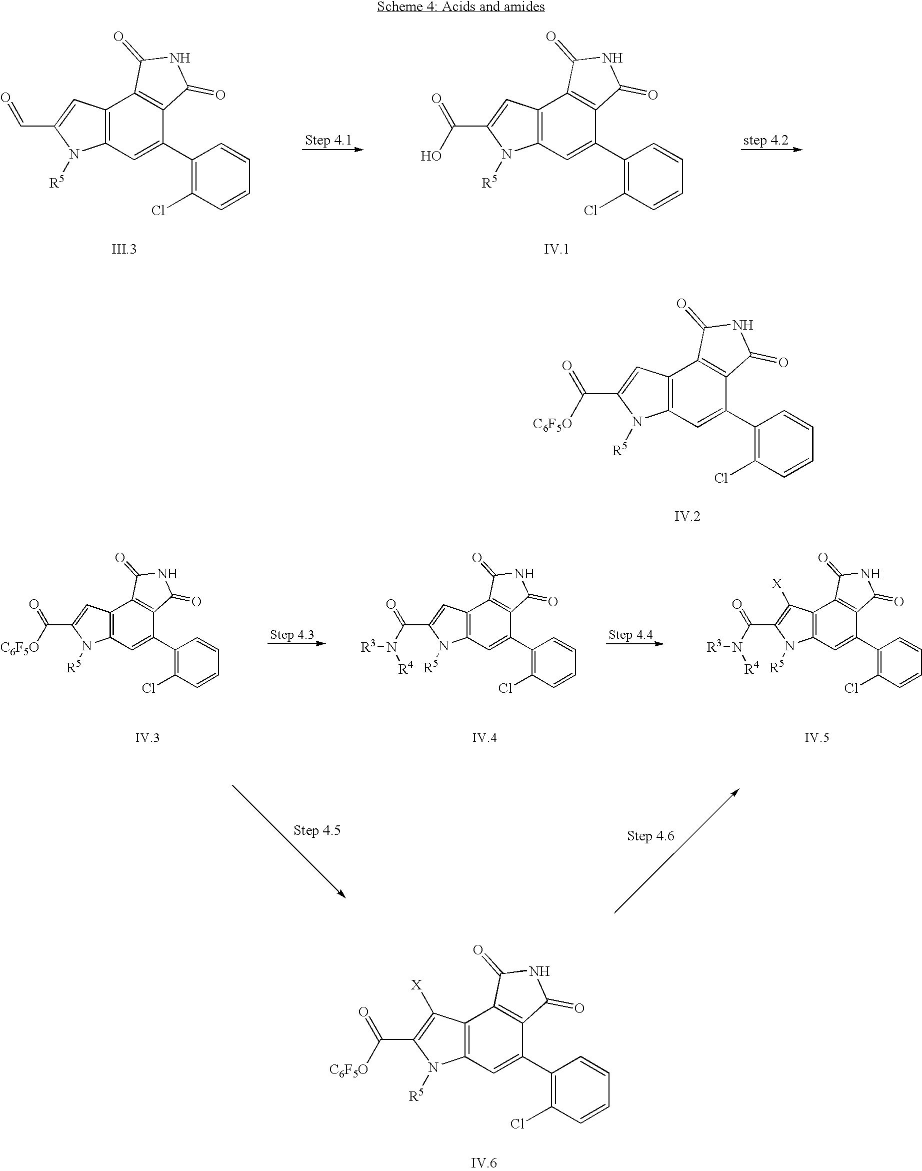

### Procedures for Scheme 4

### EXAMPLE 83

### 4-(2-Chlorophenyl)-6-methyl-1,3-dioxo-1,2,3,6-tetrahydro-2,6-diaza-as-indacene-7-carboxylic acid

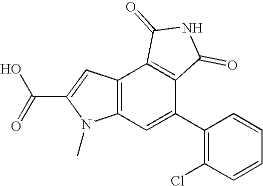

To a suspension of 4-(2-chlorophenyl)-6-methyl-1,3-dioxo-1,2,3,6-tetrahydro-2,6-diaza-as-indacene-7-carbaldehyde (0.339 g, 11.0 mmol), prepared as described in Example 72, in THF (10 mL) were added 2-methyl-2-butene (2.7 mL, 25.0 mmol) followed by t-BuOH (25 mL). Concurrently, sodium chlorite (0.904 g, 10.00 mmol) and sodium phosphate mono-basic (1.38 g, 10.00 mmol) were dissolved in water (25 mL). This aqueous solution was then added to the organic mixture and the reaction stirred at room temperature for 1 h. The reaction mixture was poured into 5% aqueous HCl (500 mL), stirred for 5 min and the solid formed was filtered. The yellow solid was washed with water and dried to give 0.124 g (35%) of the title compound; MP 301-302° C. (uncorrected); ¹H NMR (400 MHz, DMSO-D6) 8 ppm 4.11 (s, 3H) 7.45 (m, 4H) 7.55 (m, 1H) 7.88 (s, 1H) 11.04 (s, 1H); Elemental Analysis for C₁₈H₁₁ClN₂O₄.0.30H₂O % C (calc/found) 60.03/59.64, % H 3.25/3.03, % N 7.78/7.47.

### EXAMPLE 84

### 4-(2-Chlorophenyl)-6-methyl-1,3-dioxo-1,2,3,6-tetrahydro-2,6-diaza-as-indacene-7-carboxylic acid pentafluorophenyl ester

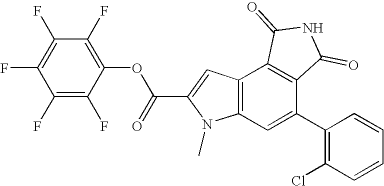

4-(2-Chlorophenyl)-6-methyl-1,3-dioxo-1,2,3,6-tetrahydro-2,6-diaza-as-indacene-7-carboxylic acid (2.56 g, 7.22 mmol), prepared as described in Example 85, and pyridine (1.71 g, 21.65 mmol) were dissolved in dimethylformamide (50 mL), under nitrogen. Pentafluorophenyltrifluoroacetate (3.03 g, 10.80 mmol) was then added and the reaction stirred overnight under a nitrogen atmosphere. The reaction was poured into ethyl acetate (250 mL) and washed five times with water (50 mL each) and three times with brine (50 mL each). The organics were dried over magnesium sulfate, filtered and concentrated. The crude solid was recrystallized from ethyl acetate/hexanes to produce 2.99 g of the title compound as a pale yellow solid; LCMS (Phenomenex Develosil Combi RP3 50×4.6 mm column, gradient method 25-2% acetonitrile eluent (0.1% formic acid) in 2.0 min with 2 min hold, MSD in ESI positive mode) 95% at 2.39 min (UV_{254 nm}); 430.9 (65), 432.9 (100), 599.8 (20); HPLC (Altima C18, 3μ, 53×7.0 mm column, gradient method 5-95% acetonitirile/water eluent (0.1% trifluoroacetic acid) in 2 min with 3 min hold, UV_{254 nm} detection 96.5% at 3.83 min; ¹H NMR (400 MHz, DMSO-D6) δ ppm 4.15 (s, 3H) 7.47 (m, 3H) 7.58 (m, 1H) 7.91 (s, 1H) 8.04 (s, 1H) 11.17 (s, 1H).

### EXAMPLE 85

### 4-(2-Chlorophenyl)-6-methyl-1,3-dioxo-1,2,3,6-tetrahydro-2,6-diaza-as-indacene-7-carboxylic acid [2-(1-methylpyrrolidin-2-yl)ethyl]amide

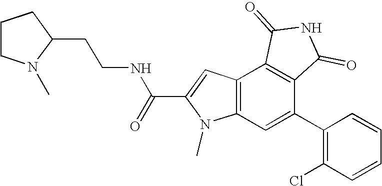

4-(2-Chlorophenyl)-6-methyl-1,3-dioxo-1,2,3,6-tetrahydro-2,6-diaza-as-indacene-7-carboxylic acid pentafluorophenyl ester (200 mg, 0.38 mmol), prepared as described in Example 84, triethylamine (78 mg, 0.77 mmol) and 2-(1-methylpyrrolidin-2-yl)-ethylamine (54 mg, 0.42 mmol) were dissolved in dimethylformamide (1 mL) and stirred at room temperature for 3 h. The reaction mixture was then poured into water (10 mL) and the yellow precipitate collected by filtration. The solid was washed with water (10 mL) and dried to produce 126 mg of the title compound as a yellow solid; ¹H NMR (400 MHz, CDCl₃) δ ppm 1.76 (s, 3H) 1.86 (m, 1H) 1.92 (m, 2H) 2.26 (s, 1H) 2.43 (d, J=3.91 Hz, 3H) 2.55 (s, 1H) 3.23 (s, 1H) 3.53 (s, 1H) 3.69 (m, 1H) 4.16 (d, J=8.79 Hz, 3H) 7.39 (m, 3H) 7.44 (s, 1H) 7.49 (s, 1H) 7.51 (m, 1H) 8.88 (s, 1H); Elemental Analysis for C₂₅H₂₅ClN₄O₃.0.42H₂O, % C (calc/found) 64.31/63.92, % H 5.44/5.13, % N 12.00/11.80.

### EXAMPLE 86

### 4-(2-Chlorophenyl)-6-methyl-1,3-dioxo-1,2,3,6-tetrahydro-2,6-diaza-as-indacene-7-carboxylic acid (3-pyrrolidin-1-ylpropyl)amide

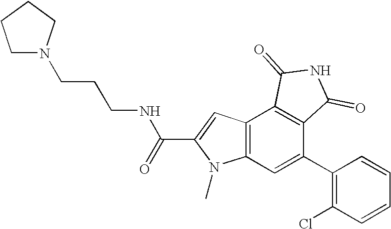

4-(2-Chlorophenyl)-6-methyl-1,3-dioxo-1,2,3,6-tetrahydro-2,6-diaza-as-indacene-7-carboxylic acid (3-pyrrolidin-1-ylpropyl)amide was prepared from 4-(2-chlorophenyl)-6-methyl-1,3-dioxo-1,2,3,6-tetrahydro-2,6-diaza-as-indacene-7-carboxylic acid pentafluorophenyl ester (200 mg, 0.38 mmol) and 3-pyrrolidin-1-ylpropylamine (78 mg, 0.77 mmol), using the procedure described in Example 85 to give 161 mg of the title compound as a yellow solid; ¹H NMR (400 MHz, DMSO-D6) δ ppm 0.94 (t, J=7.20 Hz, 1H) 1.71 (m, 7H) 2.46 (d, J=3.91 Hz, 5H) 4.08 (s, 3H) 7.44 (m, 5H) 7.54 (m, 1H) 7.81 (s, 1H) 8.97 (t, J=5.37 Hz, 1H) 10.99 (s, 1H); HPLC (Altima C18, 3μ, 53×7.0 mm column, gradient method 5-95% acetonitirile/water eluent (0.1% trifluoroacetic acid) in 2 min with 3 min hold, UV_{254 nm} detection) 92.6% at 2.61 min; LCMS (Phenomenex Develosil Combi RP3 50×4.6 mm column, gradient method 70-2% acetonitrile eluent (0.1% formic acid) in 2.0 min with 2 min hold, MSD in ESI positive mode) 92% at 1.67 min (UV_{254 nm}); API-ES 465.1 (100), 467.1 (45).

### EXAMPLE 87

### 4-(2-Chlorophenyl)-6-methyl-1,3-dioxo-1,2,3,6-tetrahydro-2,6-diaza-as-indacene-7-carboxylic acid (3-pyrrolidin-1-yl]propyl)amide

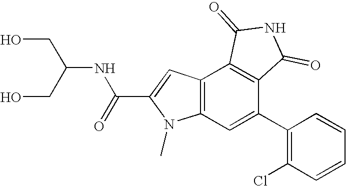

4-(2-Chlorophenyl)-6-methyl-1,3-dioxo-1,2,3,6-tetrahydro-2,6-diaza-as-indacene-7-carboxylic acid (3-pyrrolidin-1-ylpropyl)amide was prepared from 4-(2-chlorophenyl)-6-methyl-1,3-dioxo-1,2,3,6-tetrahydro-2,6-diaza-as-indacene-7-carboxylic acid pentafluorophenyl ester (100 mg, 0.19 mmol) and 2-aminopropane-1,3-diol (30 mg, 0.40 mmol), using the procedure described in Example 85 to give 71 mg of the title compound as a yellow solid; ¹H NMR (400 MHz, DMSO-D6) 8 ppm 3.51 (m, 4H) 3.97 (ddd, J=14.10, 12.03, 6.11 Hz, 1H) 4.04 (s, 3H) 4.70 (t, J=5.74 Hz, 2H) 7.42 (m, 3H) 7.53 (m, 1H) 7.59 (s, 1H) 7.79 (s, 1H) 8.43 (d, J=8.30 Hz, 1H) 10.97 (s, 1H); HPLC (Altima C18, 3μ, 53×7.0 mm column, gradient method 5-95% acetonitirile/water eluent (0.1% trifluoroacetic acid) in 2 min with 3 min hold, UV_{254 nm} detection) 96% at 8.59 min; LCMS (Phenomenex Develosil Combi RP3 50×4.6 mm column, gradient method 50-2% acetonitrile eluent (0.1% formic acid) in 2.0 min with 2 min hold, MSD in ESI positive mode) 94% at 0.97 min (UV₂₅₄ nm); API-ES 410.0 (5), 428.0 (100), 430.0 (20).

### EXAMPLE 88

### 4-(2-Chlorophenyl)-6-methyl-1,3-dioxo-1,2,3,6-tetrahydro-2,6-diaza-as-indacene-7-carboxylic acid (2-hydroxycyclopentyl)amide

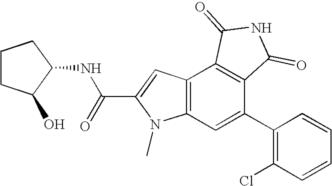

4-(2-Chlorophenyl)-6-methyl-1,3-dioxo-1,2,3,6-tetrahydro-2,6-diaza-as-indacene-7-carboxylic acid pentafluorophenyl ester (106 mg, 0.20 mmol), prepared as described in Example 84, triethylamine (62 mg, 0.61 mmol) and 2-aminocyclopentanol (84 mg, 0.83 mmol) were dissolved in dimethylformamide (1 mL) and stirred at room temperature for 3 h. The reaction mixture was then poured into water (10 mL) and the yellow precipitate collected by filtration. The solid was washed with water (10 mL) and dried to produce 163 mg of the title compound as a yellow solid; ¹H NMR (400 MHz, DMSO-D6) δ ppm 1.5 (dd, J=13.7, 6.6 Hz, 2H) 1.7 (m, 2H) 1.9 (dd, J=14.9, 6.6 Hz, 1H) 2.0 (m, 1H) 4.0 (dd, J=6.8, 4.6 Hz, 2H) 4.1 (s, 3H) 4.8 (d, J=4.2 Hz, 1H) 7.4 (m, 3H) 7.6 (m, 2H) 7.8 (s, 1H) 8.7 (d, J=7.1 Hz, 1H) 11.0 (s, 1H); Elemental Analysis for C₂₃H₂₀ClN₃O₄.0.44H₂O, % C (calc/found) 61.97/61.57, % H 4.72/4.61, % N 9.43/9.32.

### EXAMPLE 89

### 4-(2-Chlorophenyl)-6-methyl-1,3-dioxo-1,2,3,6-tetrahydro-2,6-diaza-as-indacene-7-carboxylic acid (2-dimethylaminoethyl)amide

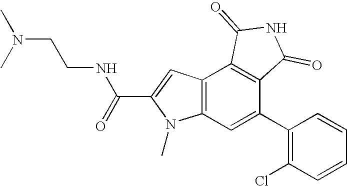

4-(2-Chlorophenyl)-6-methyl-1,3-dioxo-1,2,3,6-tetrahydro-2,6-diaza-as-indacene-7-carboxylic acid pentafluorophenyl ester (250 mg, 0.48 mmol), prepared as described in Example 84, triethylamine (146 mg, 1.44 mmol) and N,N-dimethylethylenediamine (47 mg, 0.53 mmol) were dissolved in dimethylformamide (1 mL) and stirred at room temperature for 3 h. The reaction mixture was then poured into water (10 mL) and the yellow precipitate collected by filtration. The solid was washed with water (10 mL) and dried to produce 163 mg of the title compound as a yellow solid; ¹H NMR (400 MHz, DMSO-D6) δ ppm 2.19 (s, 6H) 2.43 (t, J=6.83 Hz, 2H) 4.06 (s, 3H) 7.44 (m, 3H) 7.54 (m, 2H) 7.81 (s, 1H) 8.81 (t, J=5.61 Hz, 1H) 11.00 (s, 1H); Elemental Analysis for C₂₂H₂₁ClN₄O₃.0.25H₂O, % C (calc/found) 61.54/61.22, % H 5.05/4.87, % N 13.05/12.85.

### EXAMPLE 90

### 4-(2-Chlorophenyl)-6-methyl-1,3-dioxo-1,2,3,6-tetrahydro-2,6-diaza-as-indacene-7-carboxylic acid (2-hydroxyethyl)amide

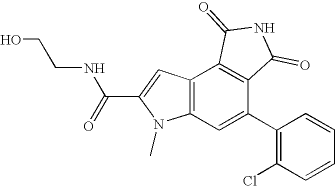

4-(2-Chlorophenyl)-6-methyl-1,3-dioxo-1,2,3,6-tetrahydro-2,6-diaza-as-indacene-7-carboxylic acid (2-hydroxyethyl)amide was prepared from 4-(2-chloro-phenyl)-6-methyl-1,3-dioxo-1,2,3,6-tetrahydro-2,6-diaza-as-indacene-7-carboxylic acid pentafluorophenyl ester (250 mg, 0.48 mmol) and ethanolamine (33 mg, 0.53 mmol), using the procedure described in Example 85 to give 298 mg of the title compound as a yellow solid; ¹H NMR (400 MHz, DMSO-D6) δ ppm 3.54 (q, J=5.94 Hz, 2H) 4.06 (s, 3H) 4.79 (t, J=5.61 Hz, 1H) 7.45 (m, 3H) 7.55 (m, 2H) 7.81 (s, 1H) 8.83 (t, J=5.49 Hz, 1H) 10.99 (s, 1H); Elemental Analysis for C₂₀H₁₆ClN₃O₄.0.20H₂O, % C (calc/found) 59.84/59.51, % H 4.12/4.08, % N 10.47/10.27.

### EXAMPLE 91

### 4-(2-Chlorophenyl)-6-methyl-1,3-dioxo-1,2,3,6-tetrahydro-2,6-diaza-as-indacene-7-carboxylic acid (2-pyrrolidin-1-ylethyl)amide

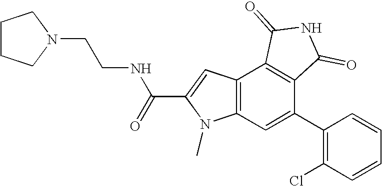

4-(2-Chlorophenyl)-6-methyl-1,3-dioxo-1,2,3,6-tetrahydro-2,6-diaza-as-indacene-7-carboxylic acid (2-pyrrolidin-1-ylethyl)amide (250 mg, 0.48 mmol) was prepared from 4-(2-chloro-phenyl)-6-methyl-1,3-dioxo-1,2,3,6-tetrahydro-2,6-diaza-as-indacene-7-carboxylic acid pentafluorophenyl ester and 1-(2-aminoethyl)pyrrolidine (62 mg, 0.54 mmol), using the procedure described in Example 85 to give 165 mg of the title compound as a yellow solid; ¹H NMR (400 MHz, DMSO-D6) δ ppm 0.94 (s, 1H) 1.68 (s, 4H) 2.61 (m, 2H) 3.39 (d, J=6.35 Hz, 4H) 4.06 (s, 3H) 7.45 (d, J=6.11 Hz, 3H) 7.54 (m, 2H) 7.81 (s, 1H) 8.85 (s, 1H) 10.98 (s, 1H); Elemental Analysis for C₂₄H₂₃ClN₄O₃, % C (calc/found) 63.67/63.34, % H 5.17/5.36, % N 12.38/12.06.

### EXAMPLE 92

### 4-(2-Chlorophenyl)-6-methyl-1,3-dioxo-1,2,3,6-tetrahydro-2,6-diaza-as-indacene-7-carboxylic acid methylamide

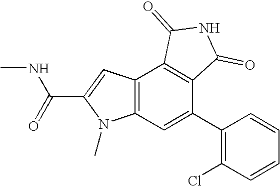

4-(2-Chlorophenyl)-6-methyl-1,3-dioxo-1,2,3,6-tetrahydro-2,6-diaza-as-indacene-7-carboxylic acid methylamide was prepared from 4-(2-chloro-phenyl)-6-methyl-1,3-dioxo-1,2,3,6-tetrahydro-2,6-diaza-as-indacene-7-carboxylic acid pentafluorophenyl ester (Amount) and methylamine (6.7 mg, 0.54 mmol, 40% W/w in water), using the procedure described in Example 85 to give 163 mg of the title compound as a yellow solid; ¹H NMR (400 MHz, DMSO-D6) δ ppm 2.80 (d, J=4.64 Hz, 3H) 4.07 (s, 3H) 7.45 (m, 4H) 7.55 (m, 1H) 7.81 (s, 1H) 8.84 (m, 1H) 10.99 (s, 1H); Elemental Analysis for C₁₉H₁₄ClN₃O₃.0.48H₂O, % C (calc/found) 60.77/60.39, % H 3.99/3.69, % N 11.19/10.99.

### EXAMPLE 93

### 4-(2-Chlorophenyl)-6-methyl-1,3-dioxo-1,2,3,6-tetrahydro-2,6-diaza-as-indacene-7-carboxylic acid amide

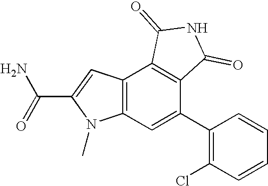

To a cooled (−25° C.) solution of 4-(2-chloro-phenyl)-6-methyl-1,3-dioxo-1,2,3,6-tetrahydro-2,6-diaza-as-indacene-7-carboxylic acid pentafluorophenyl ester (250 mg, 0.48 mmol), prepared as described in Example 84, in N,N-dimethylformamide (1 mL) was added liquid ammonia (20 drops via a cold finger condenser). The reaction mixture was stirred at −25° C. for 20 min and then the mixture was poured into water (10 mL). The solid material was filtered, washed with water (10 mL) and dried in high vacuum at 70° C. The title compound was isolated as a yellow solid (153 mg); ¹H NMR (400 MHz, DMSO-D6) δ ppm 4.07 (s, 3H) 7.44 (m, 3H) 7.56 (m, 2H) 7.65 (s, 1H) 7.81 (s, 1H) 8.32 (s, 1H) 10.99 (s, 1H); Elemental Analysis for C₁₈H₁₂ClN₃O₃.0.24H₂O, % C (calc/found) 60.38/59.99, % H 3.51/3.10, % N 11.73/11.58.

### EXAMPLE 94

### (2-{[4-(2-Chlorophenyl)-6-methyl-1,3-dioxo-1,2,3,6-tetrahydro-2,6-diaza-as-indacene-7-carbonyl]amino}ethyl)carbamic acid tert-butyl ester

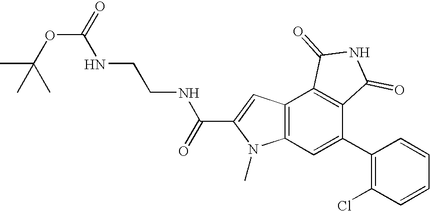

(2-{[4-(2-Chlorophenyl)-6-methyl-1,3-dioxo-1,2,3,6-tetrahydro-2,6-diaza-as-indacene-7-carbonyl]amino}ethyl)carbamic acid tert-butyl ester was prepared from 4-(2-chloro-phenyl)-6-methyl-1,3-dioxo-1,2,3,6-tetrahydro-2,6-diaza-as-indacene-7-carboxylic acid pentafluorophenyl ester (Amount) and (2-aminoethyl)carbamic acid tert-butyl ester (94 mg, 0.54 mmol), using the procedure described in Example 85 to give 218 mg of the title compound as a yellow solid;

¹H NMR (400 MHz, DMSO-D6) δ ppm 1.37 (s, 9H) 3.13 (q, J=5.94 Hz, 2H) 3.29 (m, 2H) 4.05 (s, 3H) 6.97 (t, J=5.61 Hz, 1H) 7.45 (m, 3H) 7.54 (m, 2H) 7.81 (s, 1H) 8.84 (t, J=5.37 Hz, 1H) 10.98 (s, 1H); HPLC (Altima C18, 3μ, 53×7.0 mm column, gradient method 5-95% acetonitirile/water eluent (0.1% trifluoroacetic acid) in 2 min with 3 min hold, UV_{254 nm} detection) 98.8% at 3.10 min; LCMS (Phenomenex Develosil Combi RP3 50×4.6 mm column, gradient method 50-2% acetonitrile eluent (0.1% formic acid) in 2.0 min with 2 min hold, MSD in ESI negative mode) 98% at 2.13 min (UV_{254 nm}); API-ES 421.2 (10), 495.2 (45), 497.2 (10), 541.2 (100), 543.2 (50).

### EXAMPLE 95

### 4-(2-Chlorophenyl)-6-methyl-1,3-dioxo-1,2,3,6-tetrahydro-2,6-diaza-as-indacene-7-carboxylic acid (2-aminoethyl)amide hydrochloride salt

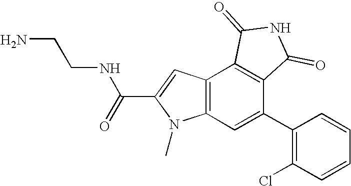

To a solution of (2-{[4-(2-chlorophenyl)-6-methyl-1,3-dioxo-1,2,3,6-tetrahydro-2,6-diaza-as-indacene-7-carbonyl]amino}ethyl)carbamic acid tert-butyl ester (62 mg, 0.12 mmol), prepared as described in Example 95, in THF was added hydrogen chloride (2 mL, 2M solution in ether). The reaction was stirred at room temperature for 12 h. The solvents were evaporated and the residue was dissolved in ethanol/0.1% HCl(aq) (1 mL/100 μl). The solvents were evaporated a second time and dried under high vacuum at 70° C. to provide 53 mg of the title compound as a yellow solid; ¹H NMR (400 MHz, DMSO-D6) δ ppm 3.03 (m, 3H) 3.54 (q, J=5.86 Hz, 2H) 4.07 (s, 4H) 7.46 (m, 4H) 7.57 (m, 2H) 7.84 (s, 1H) 7.93 (d, J=13.67 Hz, 3H) 8.98 (t, J=5.61 Hz, 1H) 11.02 (s, 1H); Elemental Analysis for C₂₀H₁₇ClN₄O₃.1.35HCl.H₂O, % C (calc/found) 53.77/54.07, % H 4.57/4.09, % N 11.97/11.59.

### EXAMPLE 96

### 4-(2-Chlorophenyl)-6-methyl-1,3-dioxo-1,2,3,6-tetrahydro-2,6-diaza-as-indacene-7-carboxylic acid dimethylamide

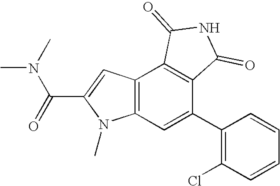

4-(2-Chlorophenyl)-6-methyl-1,3-dioxo-1,2,3,6-tetrahydro-2,6-diaza-as-indacene-7-carboxylic acid dimethylamide was prepared from 4-(2-chloro-phenyl)-6-methyl-1,3-dioxo-1,2,3,6-tetrahydro-2,6-diaza-as-indacene-7-carboxylic acid pentafluorophenyl ester (Amount) and dimethylamine (29 mg, 0.65 mmol), using the procedure described in Example 85 to give 96 mg of the title compound as a yellow solid; ¹H NMR (400 MHz, acetone) δ ppm 2.80 (m, 2H) 3.14 (s, 3H) 3.23 (s, 3H) 3.95 (s, 3H) 7.07 (s, 1H) 7.44 (m, 3H) 7.52 (m, 1H) 7.70 (s, 1H); Elemental Analysis for C₂₀H₁₆ClN₃O₃.0.70H₂O % C (calc/found) 62.47/62.08, % H 4.27/3.80, % N 10.93/10.73.

### EXAMPLE 97

### 8-Bromo-4-(2-chlorophenyl)-6-methyl-1,3-dioxo-1,2,3,6-tetrahydro-2,6-diaza-as-indacene-7-carboxylic acid pentafluorophenyl ester

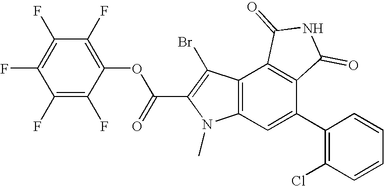

4-(2-Chlorophenyl)-6-methyl-1,3-dioxo-1,2,3,6-tetrahydro-2,6-diaza-as-indacene-7-carboxylic acid pentafluorophenyl ester (100 mg, 0.19 mmol), prepared as described in Example 84, and N-bromosuccinimide (51 mg, 0.29 mmol) were dissolved in dimethylformamide (1 mL), under nitrogen. The reaction mixture was stirred at room temperature for 2 h. The reaction mixture was poured into water (25 mL) and extracted with ethyl acetate (3 extractions of 25 mL each). The organic phases were combined and washed with water (2 times at 25 mL each), brine (25 mL) and dried over magnesium sulfate, filtered and concentrated. The crude solid was triturated with hexanes to give 105 mg of the title compound as a bright yellow solid; HPLC (Altima C18, 3μ, 53×7.0 mm column, gradient method 5-95% acetonitirile/water eluent (0.1% trifluoroacetic acid) in 10 min with 3 min hold, UV_{254 nm} detection) 99% at 5.97 min: ¹H NMR (400 MHz, DMSO-D6) δ ppm 4.12 (s, 3H) 7.47 (m, 2H) 7.57 (m, 1H) 7.95 (m, 1H) 8.12 (s, 1H) 11.22 (s, 1H); LCMS (Phenomenex Develosil Combi RP3 50×4.6 mm column, gradient method 25-2% acetonitrile/water eluents (0.1% formic acid) in 2.0 min with 2 min hold, MSD in ESI negative mode) 95% at 2.39 min (UV_{254 nm}); API-ES 388.9 (20), 430.9 (60), 432.9 (100), 434.9 (30), 599.8 (20), 631.8 (25).

### EXAMPLE 98

### 8-Chloro-4-(2-chlorophenyl)-6-methyl-1,3-dioxo-1,2,3,6-tetrahydro-2,6-diaza-as-indacene-7-carboxylic acid pentafluorophenyl ester

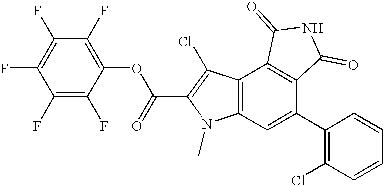

4-(2-Chlorophenyl)-6-methyl-1,3-dioxo-1,2,3,6-tetrahydro-2,6-diaza-as-indacene-7-carboxylic acid pentafluorophenyl ester (1.50 g, 2.88 mmol), prepared as described in Example 84, and N-chlorosuccinimide (0.770 g, 5.80 mmol) were dissolved in dimethylformamide (10 mL), under nitrogen. The reaction mixture was stirred at room temperature for 2 h. The reaction mixture was poured into ethyl acetate (100 mL) and washed with water (3 extractions of 25 mL each) and brine (2 times of 25 mL each). The organic phases was dried over magnesium sulfate, filtered and concentrated. The crude solid was recrystallized from ethyl acetate/hexanes to give 1.11 mg of the title compound as a bright yellow solid; LCMS (20 mm C18-MAX-RP column, gradient method 2-95% acetonitrile in water with (0.1% formic acid) in 3.3 min with 2 min hold at 95% acetonitrile in water with (0.1% formic acid), MSD in ESI positive mode) 99% at 2.46 min (UV_{210 nm}); API-ES 553.6 (100); ¹H NMR (400 MHz, DMSO-D₆) 8 ppm 4.1 (s, 3H) 7.5 (m, 3H) 7.6 (m, 1H) 8.1 (s, 1H) 11.2 (s, 1H).

### EXAMPLE 99

### 8-Bromo-4-(2-chlorophenyl)-6-methyl-1,3-dioxo-1,2,3,6-tetrahydro-2,6-diaza-as-indacene-7-carboxylic acid dimethylamide

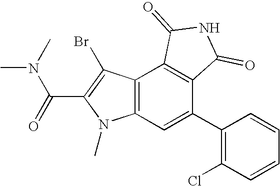

To a solution of 4-(2-chlorophenyl)-6-methyl-1,3-dioxo-1,2,3,6-tetrahydro-2,6-diaza-as-indacene-7-carboxylic acid dimethylamide (45 mg, 0.20 mmol), prepared as described in Example 96, in glacial acetic acid (5 mL) was added bromine (34 mg, 0.22 mmol) and 2,2′-azobisisobutyronitrile (5 mg, 0.029 mmol). The reaction mixture was stirred for 3 h at room temperature. The reaction mixture was then poured into water (25 mL) and extracted three times with ethyl acetate (25 mL each). The organic phases were combined and washed 5 times with water (25 mL each), three times with aqueous sodium bicarbonate (25 mL each) and brine (25 mL). The organic phase was dried over MgSO₄, filtered and concentrated. The solid was recrystallized from THF/hexanes to give the title compound as a yellow solid (74 mg); ¹H NMR (400 MHz, DMSO-D6) δ ppm 2.98 (d, J=5.62 Hz, 3H) 3.10 (s, 3H) 3.78 (s, 3H) 7.46 (m, 3H) 7.55 (d, J=8.30 Hz, 1H) 7.89 (d, J=2.69 Hz, 1H) 11.04 (s, 1H); Elemental Analysis for C₂₀H₁₅BrClN₃O₃.0.50C₄H₈O, % C (calc/found) 53.19/52.93, % H 3.86/3.65, % N 8.46/8.11.

### EXAMPLE 100

### 8-Bromo-4-(2-chlorophenyl)-6-methyl-1,3-dioxo-1,2,3,6-tetrahydro-2,6-diaza-as-indacene-7-carboxylic acid (2-hydroxyethyl)amide

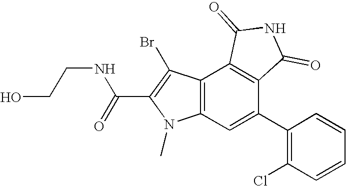

The title compound was prepared from 4-(2-chlorophenyl)-6-methyl-1,3-dioxo-1,2,3,6-tetrahydro-2,6-diaza-as-indacene-7-carboxylic acid (2-hydroxyethyl)amide (51 mg, 0.32 mmol) and bromine (128 mg, 0.32 mmol), using the procedure described in Example 99 to give 35 mg of the title compound as a yellow solid; ¹H NMR (400 MHz, DMSO-D6) δ ppm 3.39 (m, 2H) 3.56 (q, J=5.78 Hz, 2H) 3.83 (s, 3H) 4.80 (t, J=5.37 Hz, 1H) 7.47 (m, 4H) 7.54 (m, 1H) 7.91 (m, 2H) 8.85 (t, J=5.73 Hz, 1H) 11.03 (s, 1H); Melting Point 272.5-272.8° C.

### EXAMPLE 101

### 4-(2-Chlorophenyl)-6-methyl-1,3-dioxo-1,2,3,6-tetrahydro-2,6-diaza-as-indacene-7-carboxylic acid (3-diethylaminopropyl)amide

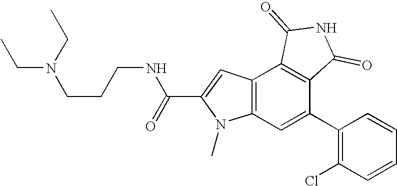

4-(2-Chlorophenyl)-6-methyl-1,3-dioxo-1,2,3,6-tetrahydro-2,6-diaza-as-indacene-7-carboxylic acid (3-diethylaminopropyl)amide was prepared from 4-(2-chlorophenyl)-6-methyl-1,3-dioxo-1,2,3,6-tetrahydro-2,6-diaza-as-indacene-7-carboxylic acid pentafluorophenyl ester (200 mg, 0.38 mmol) and N,N-diethylpropylene-1,3-diamine (55 mg, 0.42 mmol), using the procedure described in Example 84 to give 137 mg of the title compound as a yellow solid; ¹H NMR (400 MHz, CDCl₃) δ ppm 1.09 (t, J=7.20 Hz, 6H) 1.83 (s, 3H) 2.68 (m, 5H) 3.58 (m, 2H) 4.17 (s, 3H) 7.38 (m, 4H) 7.51 (m, 1H) 9.19 (s, 1H); Melting Point 215.1-217.8° C.

### EXAMPLE 102

### 8-Bromo-4-(2-chlorophenyl)-6-methyl-1,3-dioxo-1,2,3,6-tetrahydro-2,6-diaza-as-indacene-7-carboxylic acid (3-diethylaminopropyl)amide

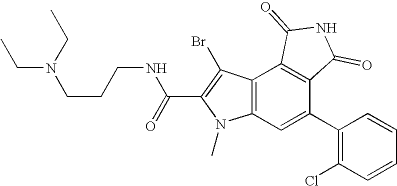

The title compound was prepared from 4-(2-Chlorophenyl)-6-methyl-1,3-dioxo-1,2,3,6-tetrahydro-2,6-diaza-as-indacene-7-carboxylic acid (3-diethylaminopropyl)amide, prepared as described in Example 101 (112 mg, 0.24 mmol) and bromine (95 mg, 0.60 mmol), using the procedure described in Example 99 to give 94 mg of the title compound as a yellow solid; ¹H NM (400 MHz, DMSO-D6) δ ppm 1.19 (m, 6H) 1.91 (d, J=13.42 Hz, 2H) 3.16 (s, 5H) 3.43 (d, J=6.10 Hz, 2H) 3.85 (s, 3H) 7.46 (m, 3H) 7.55 (ddd, J=6.47, 1.83, 1.71 Hz, 1H) 7.91 (s, 1H) 9.03 (m, 2H) 11.07 (s, 1H); Elemental Analysis for C₂₅H₂₆BrClN₄O₃.1.00HBr.C₃H₈O₂ (0.15), % C (calc/found) 47.90/47.67, % H 4.45/4.19, % N 8.78/8.39.

### EXAMPLE 103

### (3-{[4-(2-Chlorophenyl)-6-methyl-1,3-dioxo-1,2,3,6-tetrahydro-2,6-diaza-as-indacene-7-carbonyl]amino}propyl)carbamic acid tert-butyl ester

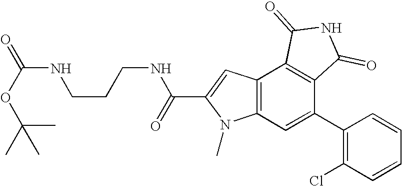

(3-{[4-(2-Chlorophenyl)-6-methyl-1,3-dioxo-1,2,3,6-tetrahydro-2,6-diaza-as-indacene-7-carbonyl]amino}propyl)carbamic acid tert-butyl ester was prepared from 4-(2-chloro-phenyl)-6-methyl-1,3-dioxo-1,2,3,6-tetrahydro-2,6-diaza-as-indacene-7-carboxylic acid pentafluorophenyl ester (200 mg, 0.38 mmol) and (3-aminopropyl)carbamic acid tert-butyl ester (74 mg, 0.42 mmol), using the procedure described in Example 85 to give 185 mg of the title compound as a yellow solid; HPLC (Altima C18, 3μ, 53×7.0 mm column, gradient method 5-95% acetonitirile/water eluent (0.1% trifluoroacetic acid) in 2 min with 3 min hold, UV_{254 nm} detection) 92% at 3.20 min; ¹H NMR (400 MHz, CDCl₃) δ ppm 1.47 (s, 9H) 1.56 (s, 2H) 1.75 (d, J=5.62 Hz, 2H) 3.28 (s, 2H) 3.51 (m, 2H) 4.12 (s, 3H) 4.87 (s, 1H) 7.39 (m, 4H) 7.51 (m, 4H); LCMS (Phenomenex Develosil Combi RP3 50×4.6 mm column, gradient method 50-2% acetonitrile/water eluent (0.1% formic acid) in 2.0 min with 2 min hold, MSD in ESI negative mode) 98% at 2.56 min (UV_{254 nm}); API-ES 509.0 (100), 511.0 (40), 555.0 (60), 557.0 (25).

### EXAMPLE 104

### (3-{[8-Bromo-4-(2-chlorophenyl)-6-methyl-1,3-dioxo-1,2,3,6-tetrahydro-2,6-diaza-as-indacene-7-carbonyl]amino}propyl)carbamic acid tert-butyl ester

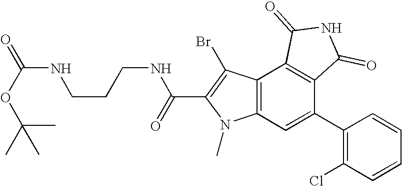

To a solution of (3-{[4-(2-chlorophenyl)-6-methyl-1,3-dioxo-1,2,3,6-tetrahydro-2,6-diaza-as-indacene-7-carbonyl]amino}propyl)carbamic acid tert-butyl ester (126 mg, 0.25 mmol), prepared as described in Example 103, in THF (5 mL) was added N-bromosuccinimide (55 mg, 0.31 mmol). The reaction mixture was poured into ethyl acetate (25 mL) and washed with water (three times with 10 mL) and brine (twice with 10 mL). The organic phase was dried over MgSO₄ and concentrated. The residue was titurated with ether, filtered and dried in vacuum oven at 60° C. to afford 126 mg of the title compound as a pale yellow solid; HPLC (Altima C18, 3μ, 53×7.0 mm column, gradient method 5-95% acetonitirile/water eluent (0.1% trifluoroacetic acid) in 2 min with 3 min hold, UV_{254 nm} detection) 96% at 3.13 min; ¹H NMR (400 MHz, DMSO-D6) δ ppm 1.38 (s, 9H) 1.67 (s, 2H) 3.05 (s, 2H) 3.83 (s, 3H) 6.87 (s, 1H) 7.46 (m, 4H) 7.54 (m, 1H) 7.89 (s, 1H) 8.86 (s, 1H) 11.04 (s, 1H); LCMS (Phenomenex Develosil Combi RP3 50×4.6 mm column, gradient method 50-2% acetonitrile/water eluent (0.1% formic acid) in 2.0 min with 2 min hold, MSD in ESI negative mode) 97% at 2.45 min (UV_{254 nm}); API-ES 586.9 (75), 588.9 (100), 590.9 (40), 632.9 (80), 634.9 (100), 635.9 (40), 670.8 (40).

### EXAMPLE 105

### 8-Bromo-4-(2-chlorophenyl)-6-methyl-1,3-dioxo-1,2,3,6-tetrahydro-2,6-diaza-as-indacene-7-carboxylic acid (3-aminopropyl)amide

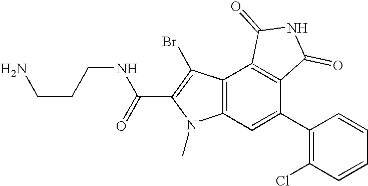

To a solution of (3-{[8-bromo-4-(2-chlorophenyl)-6-methyl-1,3-dioxo-1,2,3,6-tetrahydro-2,6-diaza-as-indacene-7-carbonyl]amino}propyl)carbamic acid tert-butyl ester (126 mg, 0.21 mmol), prepared as described in Example 104, in THF was added trifluoroacetic acid (1.48 g, 12.98 mmol). The reaction was stirred at room temperature for 12 h. The solvents were evaporated to provide 27 mg of the title compound as a yellow solid; ¹H NMR (400 MHz, DMSO-D6) δ ppm 1.87 (m, 2H) 2.93 (m, 2H) 3.40 (m, 2H) 3.84 (s, 3H) 7.46 (m, 3H) 7.55 (m, 1H) 7.90 (s, 4H) 9.04 (t, J=5.86 Hz, 1H) 11.06 (s, 1H); Elemental Analysis for C₂₁H₁₈BrClN₄O₃.1.30TFA, % C (calc/found) 44.43/44.10, % H 3.05/2.88, % N 8.78/8.71.

### EXAMPLE 106

### 8-Bromo-4-(2-chlorophenyl)-6-methyl-1,3-dioxo-1,2,3,6-tetrahydro-2,6-diaza-as-indacene-7-carboxylic acid (3-pyrrolidin-1-ylpropyl)amide

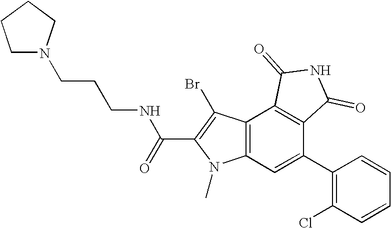

To a solution of 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.167 mmol), prepared as described in Example 97, in THF (5 mL) were added triethylamine (51 mg, 0.50 mmol) and 3-pyrrolidin-1-ylpropylamine (32 mg, 0.25 mmol). The reaction mixture was stirred in a capped two dram vial for 3 h. The reaction mixture was poured into ethyl acetate (25 mL) and washed with water (three times with 10 mL each) and brine (twice with 10 mL each). The organic phase was dried over MgSO₄, concentrated and dried in high vacuum at 70° C. The title compound was isolated as a yellow solid (55 mg); ¹H NMR (400 MHz, DMSO-D6) δ ppm 2.21 (m, 2H) 2.53 (m, 2H) 3.11 (ddd, J=13.43, 6.59, 6.35 Hz, 2H) 3.24 (dt, J=13.37, 7.85 Hz, 2H) 3.37 (m, 2H) 3.57 (m, 2H) 3.84 (m, 3H) 4.75 (m, 1H) 7.44 (m, 4H) 7.54 (m, 1H) 7.92 (m, 1H) 9.41 (d, J=7.08 Hz, 1H) 11.06 (s, 1H); Elemental Analysis for C₁₉H₁₃BrClN₃O₃.0.65H₂O, % C (calc/found) 54.05/54.41, % H 4.59/3.84, % N 10.08/9.69.

### EXAMPLE 107

### 8-Bromo-4-(2-chlorophenyl)-6-methyl-1,3-dioxo-1,2,3,6-tetrahydro-2,6-diaza-as-indacene-7-carboxylic acid [2-(1-methylpyrrolidin-2-yl)ethyl]amide

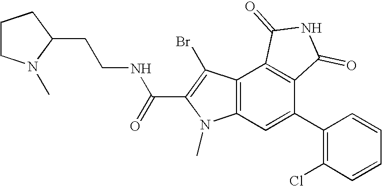

To a solution of 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol), prepared as described in Example 97, in DMF (1 mL) were added triethylamine (51 mg, 0.50 mmol) and 2-(1-methylpyrrolidin-2-yl)ethylamine (32 mg, 0.25 mmol). The reaction mixture was stirred in a sealed vial for 3 h. The reaction mixture was poured into water (25 mL) and filtered. The title compound was isolated as a yellow solid (48 mg); ¹H NMR (400 MHz, DMSO-D6) δ ppm 1.45 (m, 2H) 1.63 (m, 2H) 1.93 (m, 2H) 2.07 (s, 1H) 2.23 (s, 4H) 2.94 (d, J=5.13 Hz, 1H) 3.35 (m, 2H) 3.83 (s, 3H) 7.45 (m, 3H) 7.55 (ddd, J=4.76, 3.17, 1.59 Hz, 1H) 7.88 (s, 1H) 8.91 (t, J=5.74 Hz, 1H) 11.05 (s, 1H); HPLC (Altima C18, 3μ, 53×7.0 mm column, gradient method 5-95% acetonitirile/water eluent (0.1% trifluoroacetic acid) in 10 min with 3 min hold, UV_{254 nm} detection) 95% at 5.61 min; LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 35% at 1.35 min, 63% at 1.48 min (broad, sloping peak) (UV_{254 nm}); API-ES 542.9 (75), 544.9 (100), 546.9 (25).

### EXAMPLE 108

### 8-Bromo-4-(2-chlorophenyl)-6-methyl-1,3-dioxo-1,2,3,6-tetrahydro-2,6-diaza-as-indacene-7-carboxylic acid (2-dimethylaminoethyl)amide

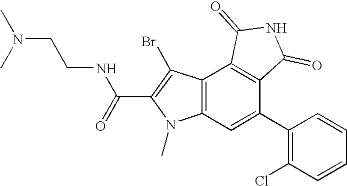

8-Bromo-4-(2-chlorophenyl)-6-methyl-1,3-dioxo-1,2,3,6-tetrahydro-2,6-diaza-as-indacene-7-carboxylic acid (2-dimethylaminoethyl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol) and N,N-dimethylethylenediamine (22 mg, 0.25 mmol), using the procedure described in Example 107 to give 55 mg of the title compound as a yellow solid; ¹H NMR (400 MHz, DMSO-D6) δ ppm 2.19 (m, 6H) 2.44 (t, J=6.72 Hz, 2H) 3.34 (m, 2H) 3.41 (m, 2H) 3.84 (s, 3H) 7.45 (m, 3H) 7.54 (m, 1H) 7.89 (m, 1H) 8.81 (t, J=5.62 Hz, 1H) 11.04 (s, 1H); HPLC (Altima C18, 3μ, 53×7.0 mm column, gradient method 5-95% acetonitirile/water eluent (0.1% trifluoroacetic acid) in 10 min with 3 min hold, UV_{254 nm} detection) 91% at 5.41 min; LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 93% at 1.03 min (UV_{254 nm}); API-ES 504.9 (100), 506.9 (25).

### EXAMPLE 109

### 8-Bromo-4-(2-chlorophenyl)-6-methyl-1,3-dioxo-1,2,3,6-tetrahydro-2,6-di aza-as-indacene-7-carboxylic acid (3-amino-2-hydroxypropyl)amide

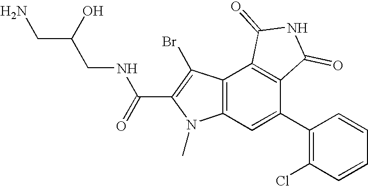

8-Bromo-4-(2-chlorophenyl)-6-methyl-1,3-dioxo-1,2,3,6-tetrahydro-2,6-diaza-as-indacene-7-carboxylic acid (3-amino-2-hydroxypropyl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol) and 1,3-diaminopropan-2-ol (23 mg, 0.25 mmol), using the procedure described in Example 107 to give 41 mg of the title compound as a yellow solid; ¹H NMR (400 MHz, DMSO-D6) δ ppm 3.42 (s, 2H) 3.51 (d, J=13.68 Hz, 2H) 3.87 (s, 3H) 3.93 (s, 1H) 5.27 (d, J=5.37 Hz, 1H) 7.45 (m, 4H) 7.55 (m, 1H) 7.89 (d, J=5.37 Hz, 1H) 8.94 (s, 1H) 11.04 (s, 1H); HPLC (Altima C18, 3μ, 53×7.0 mm column, gradient method 5-95% acetonitirile/water eluent (0.1% trifluoroacetic acid) in 10 min with 3 min hold, UV_{254 nm} detection) 88% at 7.79 min; LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 80% at 2.33 min (solvent impurities) (UV₂₅₄ nm); API-ES 101.2 (100), 122.2 (75), 504.9 (40).

### EXAMPLE 110

### 8-Bromo-4-(2-chlorophenyl)-6-methyl-1,3-dioxo-1,2,3,6-tetrahydro-2,6-diaza-as-indacene-7-carboxylic acid (3-acetylaminopropyl)amide

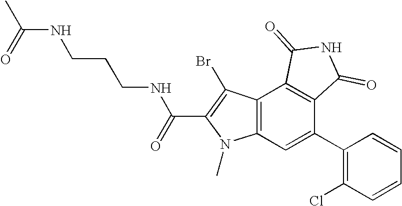

8-Bromo-4-(2-chlorophenyl)-6-methyl-1,3-dioxo-1,2,3,6-tetrahydro-2,6-diaza-as-indacene-7-carboxylic acid (3-acetylaminopropyl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol) and N-(3-aminopropyl)acetamide (25 mg, 0.25 mmol), using the procedure described in Example 107 to give 47 mg of the title compound as a yellow solid; ¹H NMR (400 MHz, DMSO-D6) δ ppm 1.80 (d, J=7.08 Hz, 3H) 3.26 (m, 2H) 3.37 (m, 2H) 3.83 (s, 3H) 7.45 (m, 4H) 7.54 (m, 1H) 7.90 (m, 1H) 7.95 (t, J=5.62 Hz, 1H) 8.91 (t, J=5.74 Hz, 1H) 10.97 (s, 1H); HPLC (Altima C18, 3μ, 53×7.0 mm column, gradient method 5-95% acetonitirile/water eluent (0.1% trifluoroacetic acid) in 10 min with 3 min hold, UV_{254 nm} detection) 97% at 5.10 min; LCMS (Phenomenex Develosil Combi RP3 50×4.6 mm column, gradient method 25-2% acetonitrile/water eluent (0.1% formic acid) in 2.0 min with 2 min hold, MSD in ESI positive mode) 100% at 0.73 min (UV_{254 nm}); API-ES 516.9 (80), 518.9 (100), 520.9 (20).

### EXAMPLE 111

### 8-Bromo-4-(2-chlorophenyl)-6-methyl-1,3-dioxo-1,2,3,6-tetrahydro-2,6-diaza-as-indacene-7-carboxylic acid (tetrahydrofuran-2-ylmethyl)amide

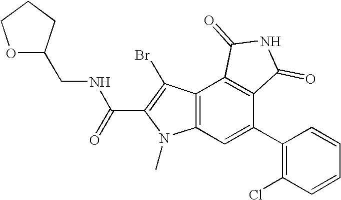

8-Bromo-4-(2-chlorophenyl)-6-methyl-1,3-dioxo-1,2,3,6-tetrahydro-2,6-diaza-as-indacene-7-carboxylic acid (tetrahydrofuran-2-ylmethyl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol) and racemic tetrahydrofuran-2-yl-methylamine (34 mg, 0.33 mmol), using the procedure described in Example 107 to give 66 mg of the title compound as a yellow solid; ¹H NMR (400 MHz, DMSO-D6) δ ppm 1.67 (m, 1H) 1.86 (m, 2H) 1.97 (m, 1H) 3.40 (m, 2H) 3.65 (m, 1H) 3.81 (s, 3H) 4.02 (m, 1H) 7.45 (m, 3H) 7.55 (dd, J=7.94, 1.59 Hz, 1H) 7.88 (s, 1H) 8.96 (t, J=5.86 Hz, 1H) 11.04 (s, 1H); HPLC (Altima C18, 3μ, 53×7.0 mm column, gradient method 5-95% acetonitirile/water eluent (0.1% trifluoroacetic acid) in 10 min with 3 min hold, UV_{254 nm} detection) 94% at 6.60 min; LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 24% at 1.95 min, 68% at 2.05 min (UV_{254 nm}); API-ES 515.9 (80), 517.9 (100), 519.9 (25).

### EXAMPLE 112

### 8-Bromo-4-(2-chlorophenyl)-6-methyl-1,3-dioxo-1,2,3,6-tetrahydropyrrolo[3,4-e]indole-7-carboxylic acid (2-hydroxy-1-hydroxymethylethyl)amide

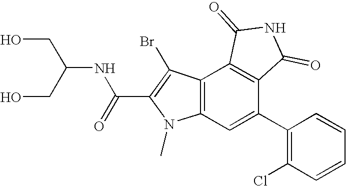

The title compound was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol) and 2-aminopropane-1,3-diol (30 mg, 0.30 mmol), using the procedure described in Example 107 to give 58 mg of the title compound as a yellow solid; ¹H NMR (400 MHz, DMSO-D6) δ ppm 3.55 (m, 4H) 3.83 (s, 3H) 4.02 (m, 1H) 4.76 (t, J=5.37 Hz, 2H) 7.44 (m, 3H) 7.54 (m, 1H) 7.88 (m, 1H) 8.59 (d, J=8.79 Hz, 1H) 11.03 (s, 1H); HPLC (Altima C18, 3μ, 53×7.0 mm column, gradient method 5-95% acetonitirile/water eluent (0.1% trifluoroacetic acid) in 10 min with 3 min hold, UV_{254 nm} detection) 92% at 4.67 min; LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 17% at 0.81 min, 76% at 0.89 min (broad sloping peak) (UV_{254 nm}); API-ES (identical spectra for each) 428.0 (25), 505.9 (90), 507.9 (100), 509.9 (40).

### EXAMPLE 113

### 8-Bromo-4-(2-chlorophenyl)-6-methyl-1,3-dioxo-1,2,3,6-tetrahydro-2,6-diaza-as-indacene-7-carboxylic acid [2-(2-hydroxyethoxy)ethyl]amide

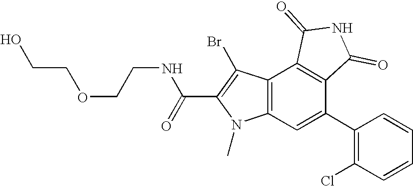

8-Bromo-4-(2-chlorophenyl)-6-methyl-1,3-dioxo-1,2,3,6-tetrahydro-2,6-diaza-as-indacene-7-carboxylic acid [2-(2-hydroxyethoxy)ethyl]amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol) and 2-(2-aminoethoxy)ethanol (35 mg, 0.33 mmol), using the procedure described in Example 107 to give 55 mg of the title compound as a yellow solid; ¹H NMR (400 MHz, DMSO-D6) δ ppm 3.49 (m, 6H) 3.58 (m, 2H) 3.83 (s, 3H) 4.59 (s, 1H) 7.45 (s, 3H) 7.54 (s, 1H) 7.88 (s, 1H) 8.92 (s, 1H) 11.05 (s, 1H); HPLC (Altima C18, 3μ, 53×7.0 mm column, gradient method 5-95% acetonitirile/water eluent (0.1% trifluoroacetic acid) in 10 min with 3 min hold, UV_{254 nm} detection) 94% at 5.22 min; LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 29% at 1.01 min, 69% at 1.10 min (broad peak) (UV_{254 nm}); API-ES (identical for each) 442.0 (20), 519.9 (90), 521.9 (100), 523.9 (35).

### EXAMPLE 114

### 8-Bromo-4-(2-chlorophenyl)-6-methyl-1,3-dioxo-1,2,3,6-tetrahydro-2,6-diaza-as-indacene-7-carboxylic acid (1,1-dioxotetrahydro-1λ⁶-thiophen-3-yl)amide

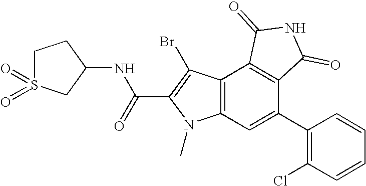

8-Bromo-4-(2-chlorophenyl)-6-methyl-1,3-dioxo-1,2,3,6-tetrahydro-2,6-diaza-as-indacene-7-carboxylic acid (1,1-dioxo-tetrahydro-1λ⁶-thiophen-3-yl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (100 mg, 0.17 mmol) and 1,1-dioxotetrahydro-1λ⁶-thiophen-3-ylamine (35 mg, 0.33 mmol), using the procedure described in Example 107 to give 48 mg of the title compound as a yellow solid; ¹H NMR (400 MHz, DMSO-D6) δ ppm 2.21 (m, 1H) 2.52 (m, 2H) 3.11 (m, 1H) 3.24 (dt, J=13.37, 7.85 Hz, 1H) 3.37 (m, 1H) 3.56 (d, J=8.06 Hz, 1H) 3.85 (s, 3H) 4.75 (m, 1H) 7.44 (m, 3H) 7.55 (td, J=3.97, 1.83 Hz, 1H) 7.91 (m, 1H) 9.41 (d, J=6.59 Hz, 1H) 11.06 (s, 1H); HPLC (Altima C18, 3μ, 53×7.0 mm column, gradient method 5-95% acetonitirile/water eluent (0.1% trifluoroacetic acid) in 2 min with 3 min hold, UV_{254 nm} detection) 100% at 2.85 min; LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 23% at 1.30 min, 77% at 1.41 min (broad peak) (UV_{254 nm}); API-ES 106.2 (100), 547.5 (25), 549.5 (40).

### EXAMPLE 115

### 8-Bromo-4-(2-chlorophenyl)-6-methyl-1,3-dioxo-1,2,3,6-tetrahydro-2,6-diaza-as-indacene-7-carboxylic acid (2-hydroxyphenyl)amide

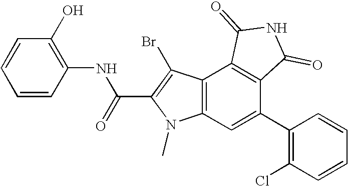

8-Bromo-4-(2-chlorophenyl)-6-methyl-1,3-dioxo-1,2,3,6-tetrahydro-2,6-diaza-as-indacene-7-carboxylic acid (2-hydroxyphenyl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (102 mg, 0.17 mmol) and 2-aminophenol (41 mg, 0.37 mmol), using the procedure described in Example 107 to give 48 mg of the title compound as a yellow solid; ¹H NMR (400 MHz, DMSO-D6) δ ppm 3.9 (s, 3H) 6.9 (m, 1H) 6.9 (dd, J=8.1, 1.2 Hz, 1H) 7.0 (td, J=7.7, 1.5 Hz, 1H) 7.5 (m, 3H) 7.6 (m, 1H) 7.9 (s, 1H) 8.0 (dd, J=7.9, 1.6 Hz, 1H) 9.9 (s, 1H) 10.0 (s, 1H) 11.1 (s, 1H); Elemental Analysis for C₂₄H₁₅BrClN₃O₄% C (calc/found) 54.93/54.88, % H 2.88/2.52, % N 8.01/7.81.

### EXAMPLE 116

### 8-Bromo-4-(2-chlorophenyl)-6-methyl-1,3-dioxo-1,2,3,6-tetrahydro-2,6-diaza-as-indacene-7-carboxylic acid (2-hydroxycyclopentyl)amide

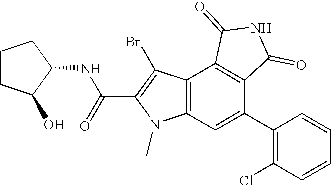

8-Bromo-4-(2-chlorophenyl)-6-methyl-1,3-dioxo-1,2,3,6-tetrahydro-2,6-diaza-as-indacene-7-carboxylic acid (2-hydroxycyclopentyl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (99 mg, 0.16 mmol) and 2-aminocyclopentanol (62 mg, 0.33 mmol), using the procedure described in Example 107 to give 66 mg of the title compound as a yellow solid; ¹H NMR (400 MHz, DMSO-D6) δ ppm 1.50 (s, 2H) 1.67 (s, 2H) 1.84 (s, 1H) 2.04 (s, 1H) 3.82 (s, 3H) 4.06 (s, 2H) 4.86 (d, J=3.91 Hz, 1H) 7.46 (m, 2H) 7.55 (d, J=7.82 Hz, 1H) 7.88 (s, 1H) 8.77 (d, J=7.08 Hz, 1H) 11.03 (s, 1H); Elemental Analysis for C₂₃H₁₉BrClN₃O₄, % C (calc/found) 53.46/53.40, % H 3.71/3.58, % N 8.13/7.97.

### EXAMPLE 117

### 8-Bromo-4-(2-chlorophenyl)-6-methyl-1,3-dioxo-1,2,3,6-tetrahydro-2,6-diaza-as-indacene-7-carboxylic acid methylamide

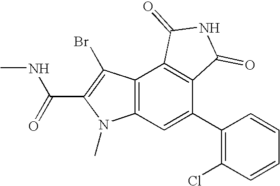

8-Bromo-4-(2-chlorophenyl)-6-methyl-1,3-dioxo-1,2,3,6-tetrahydro-2,6-diaza-as-indacene-7-carboxylic acid methylamide was prepared from 4′-(2-chlorophenyl)-6-methyl-1,3-dioxo-1,2,3,6-tetrahydro-2,6-diaza-as-indacene-7-carboxylic acid methylamide (99 mg, 0.16 mmol), itself prepared as described in Example 92, and N-bromosuccinimide (62 mg, 0.33 mmol), using the procedure described in Example 104 to give 38 mg of the title compound as a yellow solid; ¹H NMR (400 MHz, DMSO-D6) δ ppm 2.86 (d, J=4.64 Hz, 3H) 3.83 (s, 3H) 7.45 (m, 3H) 7.54 (m, 1H) 7.88 (s, 1H) 8.81 (d, J=4.88 Hz, 1H) 11.04 (s, 1H); Elemental Analysis for C₁₉H₁₃BrClN₃O₃.0.06H₂O, % C (calc/found) 50.97/50.57, % H 2.95/2.87, % N 9.38/9.21.

### EXAMPLE 118

### 8-Bromo-4-(2-chlorophenyl)-6-methyl-1,3-dioxo-1,2,3,6-tetrahydro-2,6-diaza-as-indacene-7-carboxylic acid amide

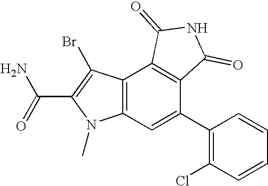

8-Bromo-4-(2-chlorophenyl)-6-methyl-1,3-dioxo-1,2,3,6-tetrahydro-2,6-diaza-as-indacene-7-carboxylic acid amide was prepared from 4-(2-Chlorophenyl)-6-methyl-1,3-dioxo-1,2,3,6-tetrahydro-2,6-diaza-as-indacene-7-carboxylic acid amide, prepared as described in Example 93 (108 mg, 0.29 mmol), using the procedure described in Example 104 to give 33 mg of the title compound as a yellow solid; ¹H NMR (400 MHz, DMSO-D6) δ ppm 3.86 (s, 3H) 7.45 (m, 3H) 7.54 (m, 1H) 7.88 (s, 1H) 8.25 (d, J=10.01 Hz, 2H) 11.03 (s, 1H); Elemental Analysis for C₁₈H₁₁BrClN₃O₃.0.35H₂O, % C (calc/found) 49.25/48.86, % H 2.69/2.41, % N 9.57/9.41.

### EXAMPLE 119

### 8-Bromo-4-(2-chlorophenyl)-6-methyl-1,3-dioxo-1,2,3,6-tetrahydro-2,6-diaza-as-indacene-7-carboxylic acid (2-aminoethyl)amide hydrobromide salt

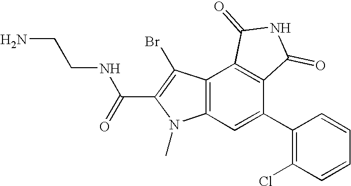

8-Bromo-4-(2-chlorophenyl)-6-methyl-1,3-dioxo-1,2,3,6-tetrahydro-2,6-diaza-as-indacene-7-carboxylic acid (2-aminoethyl)amide hydrobromide salt was prepared from (2-{[4-(2-Chlorophenyl)-6-methyl-1,3-dioxo-1,2,3,6-tetrahydro-2,6-diaza-as-indacene-7-carbonyl]amino}ethyl)carbamic acid tert-butyl ester (153 mg, 0.31 mmol) described in Example 94, using the procedure described in Example 104 to give 56 mg of the title compound as a yellow solid; ¹H NMR (400 MHz, DMSO-D6) δ ppm 3.03 (s, 2H) 3.59 (m, 2H) 3.88 (s, 3H) 7.46 (m, 3H) 7.55 (m, 1H) 7.84 (s, 3H) 7.92 (s, 1H) 8.99 (t, J=5.73 Hz, 1H) 11.07 (s, 1H); Elemental Analysis for C₂₀H₁₆BrClN₄O₃.1.13HBr 0.08 C₂H₅OH, % C (calc/found) 42.42/42.78, % H 3.11/2.79, % N 9.81/9.42.

### EXAMPLE 120

### 8-Bromo-4-(2-chlorophenyl)-6-methyl-1,3-dioxo-1,2,3,6-tetrahydro-2,6-diaza-as-indacene-7-carboxylic acid (2-pyrrolidin-1-ylethyl)amide hydrobromide salt

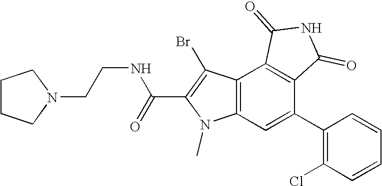

To a solution of 4-(2-chlorophenyl)-6-methyl-1,3-dioxo-1,2,3,6-tetrahydro-2,6-diaza-as-indacene-7-carboxylic acid (2-pyrrolidin-1-ylethyl)amide (146 mg, 0.32 mmol), prepared as described in Example 91, in glacial acetic acid (1 mL) was added bromine (57 mg, 0.35 mmol). The reaction mixture was stirred at room temperature for 15 min. The solid product was filtered off and washed twice with water (10 mL each). The solids were dried in high vacuum at 70° C. to produce 36 mg of the title compound as a bright yellow solid; ¹H NMR (400 MHz, DMSO-D6) δ ppm 1.75 (ddd, J=6.47, 3.42, 3.30 Hz, 2H) 1.88 (d, J=17.83 Hz, 2H) 2.04 (d, J=0.98 Hz, 2H) 3.10 (s, 2H) 3.59 (t, J=6.59 Hz, 4H) 3.69 (s, 3H) 3.86 (d, J=7.82 Hz, 3H) 7.47 (m, 3H) 7.56 (m, 1H) 7.92 (s, 1H) 9.09 (t, J=5.74 Hz, 1H) 9.54 (s, 1H) 11.07 (s, 1H); Elemental Analysis for C₂₄H₂₂BrClN₄O₃.1.13HBr.0.45C₂H₄O₂, % C (calc/found) 45.73/46.02, % H 3.85/3.72, % N 8.57/8.23.

### EXAMPLE 121

### 8-Bromo-4-(2-chlorophenyl)-6-methyl-1,3-dioxo-1,2,3,6-tetrahydro-2,6-diaza-as-indacene-7-carboxylic acid (3-hydroxypyridin-2-yl)amide

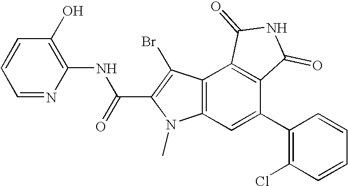

8-Bromo-4-(2-chlorophenyl)-6-methyl-1,3-dioxo-1,2,3,6-tetrahydro-2,6-diaza-as-indacene-7-carboxylic acid (3-hydroxypyridin-2-yl)amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (99 mg, 0.16 mmol) and 2-aminopyridin-3-ol (62 mg, 0.33 mmol), using the procedure described in Example 107 to give 48 mg of the title compound as a yellow solid; ¹H NMR (400 MHz, DMSO-D6) δ ppm 3.94 (s, 3H) 6.86 (m, 1H) 6.92 (dd, J=8.06, 1.22 Hz, 1H) 7.05 (td, J=7.69, 1.47 Hz, 1H) 7.46 (m, 3H) 7.56 (m, 1H) 7.93 (s, 1H) 7.98 (dd, J=7.82, 1.47 Hz, 1H) 9.94 (s, 1H) 10.02 (s, 1H) 11.06 (s, 1H); Elemental Analysis for C₂₃H₁₄BrClN₄O₄, % C (calc/found) 52.54/52.60, % H 2.68/2.69, % N 10.66/1.035.

### EXAMPLE 122

### 8-Bromo-4-(2-chlorophenyl)-6-methyl-1,3-dioxo-1,2,3,6-tetrahydro-2,6-diaza-as-indacene-7-carboxylic acid {3-[2-(3-aminopropoxy)ethoxy]propyl}amide

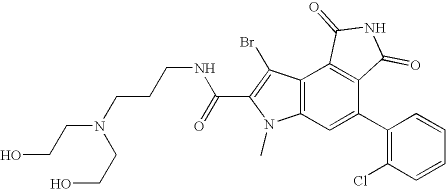

8-Bromo-4-(2-chlorophenyl)-6-methyl-1,3-dioxo-1,2,3,6-tetrahydro-2,6-diaza-as-indacene-7-carboxylic acid {3-[2-(3-aminopropoxy)ethoxy]propyl}amide was prepared from 8-bromo-4-(2-chlorophenyl)-6-methyl-1,3-dioxo-1,2,3,6-tetrahydro-2,6-diaza-as-indacene-7-carboxylic acid pentafluorophenyl ester (99 mg, 0.16 mmol) and 2-[(3-aminopropyl)-(2-hydroxyethyl)amino]ethanol (50 mg, 0.30 mmol), using the procedure described in Example 107 to give 66 mg of the title compound as a yellow solid; ¹H NMR (400 MHz, DMSO-D6) δ ppm 1.98 (s, 2H) 3.27 (dd, J=15.51, 4.76 Hz, under residual water peak) 3.31 (s, under residual water peak) 3.42 (d, J=6.35 Hz, under residual water peak) 3.76 (s, 4H) 3.85 (s, 3H) 5.32 (s, 2H) 7.46 (m, 3H) 7.55 (dt, J=6.29, 1.74 Hz, 1H) 7.91 (s, 1H) 9.01 (t, J=5.74 Hz, 1H) 9.13 (s, 1H) 11.06 (s, 1H); HPLC (Altima C18, 3μ, 53×7.0 mm column, gradient method 5-95% acetonitirile/water eluent (0.1% trifluoroacetic acid) in 2 min with 3 min hold, UV_{254 nm} detection) 97% at 2.59 min; LCMS (Phenomenex Develosil Combi RP3 50×4.6 mm column, gradient method 70-2% acetonitrile/water eluent (0.1% formic acid) in 2.0 min with 2 min hold, MSD in ESI positive mode) 16% at 0.94 min, 78% at 1.05 min (broad peak) (UV_{254 nm}); API-ES 576.9 (20), 578.9 (25), 579.9 (10).

### EXAMPLE 123

### 8-Bromo-4-(2-chlorophenyl)-6-methyl-1,3-dioxo-1,2,3,6-tetrahydro-2,6-diaza-as-indacene-7-carboxylic acid {2-[bis-(2-hydroxyethyl)amino]ethyl}amide

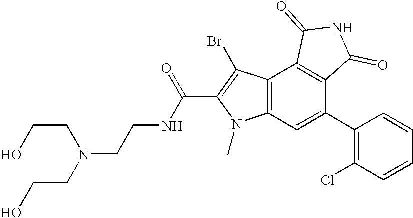

8-Bromo-4-(2-chlorophenyl)-6-methyl-1,3-dioxo-1,2,3,6-tetrahydro-2,6-diaza-as-indacene-7-carboxylic acid {2-[bis-(2-hydroxyethyl)amino]ethyl}amide was prepared from 8-bromo-4-(2-chloro-phenyl)-6-methyl-1,3-dioxo-1,2,3,6-tetrahydro-2,6-diaza-as-indacene-7-carboxylic acid pentafluorophenyl ester (100 mg, 0.16 mmol) and 2-[(2-aminoethyl)-(2-hydroxyethyl)amino]ethanol (50 mg, 0.30 mmol), using the procedure described in Example 107 to give 66 mg of the title compound as a yellow solid; ¹H NMR (400 MHz, DMSO-D6) δ ppm 3.45 (s, coincident with residual water peak) 3.77 (m, 7H) 3.86 (s, 3H) 5.37 (s, 1H) 7.45 (m, 3H) 7.55 (m, J=8.15, 2.08, 1.85, 1.85 Hz, 1H) 7.92 (s, 1H) 9.11 (t, J=5.62 Hz, 1H) 9.27 (s, 1H) 11.07 (s, 1H); HPLC (Altima C18, 3μ, 53×7.0 mm column, gradient method 5-95% acetonitirile/water eluent (0.1% trifluoroacetic acid) in 2 min with 3 min hold, UV_{254 nm} detection) 95% at 2.49 min; LCMS (Phenomenex Develosil Combi RP3 50×4.6 mm column, gradient method 70-2% acetonitrile/water eluent (0.1% formic acid) in 2.0 min with 2 min hold, MSD in ESI positive mode) 20% at 0.91 min, 76% at 1.03 min (broad peak) (UV₂₅₄ nm); API-ES 562.9 (80), 564.9 (100), 566.9 (25).

### EXAMPLE 124

### 8-Chloro-4-(2-chlorophenyl)-6-methyl-1,3-dioxo-1,2,3,6-tetrahydro-2,6-diaza-as-indacene-7-carboxylic acid (2-hydroxy-1-hydroxymethylethyl)amide

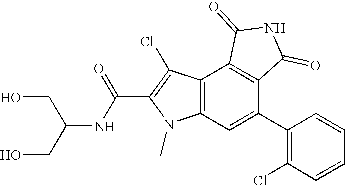

To a solution of 8-chloro-4-(2-chlorophenyl)-6-methyl-1,3-dioxo-1,2,3,6-tetrahydro-2,6-diaza-as-indacene-7-carboxylic acid pentafluorophenyl ester (102 mg, 0.18 mmol), prepared as described in Example 98, in DMF (1 mL) was added triethylamine (51 mg, 0.50 mmol) and 2-aminopropane-1,3-diol (32 mg, 0.25 mmol). The reaction mixture was stirred in a sealed vial for 3 h. The reaction mixture was poured into water (25 mL) and filtered. The title compound was isolated as a yellow solid (41 mg); ¹H NMR (400 MHz, DMSO-D6) δ ppm 3.5 (m, 4H) 3.9 (s, 3H) 4.0 (dt, J=8.2, 5.8 Hz, 1H) 4.8 (t, J=5.6 Hz, 2H) 7.5 (m, 3H) 7.5 (m, 1H) 7.9 (s, 1H) 8.5 (d, J=8.5 Hz, 1H) 11.0 (s, 1H); Elemental Analysis for C₂₁H₁₇Cl₂N₃O₅.0.20H₂O, % C (calc/found) 54.14/53.78, % H 3.76/3.47, % N 9.02/8.71.

### EXAMPLE 125

### 8-Chloro-4-(2-chlorophenyl)-6-methyl-1,3-dioxo-1,2,3,6-tetrahydro-2,6-diaza-as-indacene-7-carboxylic acid (2-hydroxyethyl)amide

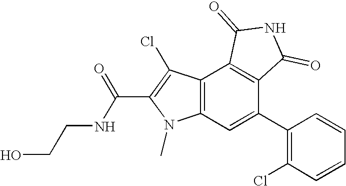

The title compound was prepared from 8-chloro-4-(2-chlorophenyl)-6-methyl-1,3-dioxo-1,2,3,6-tetrahydro-2,6-diaza-as-indacene-7-carboxylic acid pentafluorophenyl ester (102 mg, 0.18 mmol) and ethanolamine (33 mg, 0.37 mmol), using the procedure described in Example 124 to give 51 mg of the title compound as a yellow solid; ¹H NMR (400 MHz, DMSO-D6) δ ppm 2.7 (s, 1H) 2.9 (s, 1H) 3.4 (td, J=6.0, 1.5 Hz, 2H) 3.6 (q, J=6.0 Hz, 2H) 3.8 (s, 3H) 4.8 (t, J=5.4 Hz, 1H) 7.5 (m, 3H) 7.5 (m, 1H) 7.9 (s, 1H) 8.8 (t, J=5.7 Hz, 1H) 11.1 (s, 1H); Elemental Analysis for C₂₀H₁₅Cl₂N₃O₄.0.36H₂O-0.05 C₃H₇NO, % C (calc/found) 54.71/54.36, % H 3.66/3.55, % N 9.66/10.02.

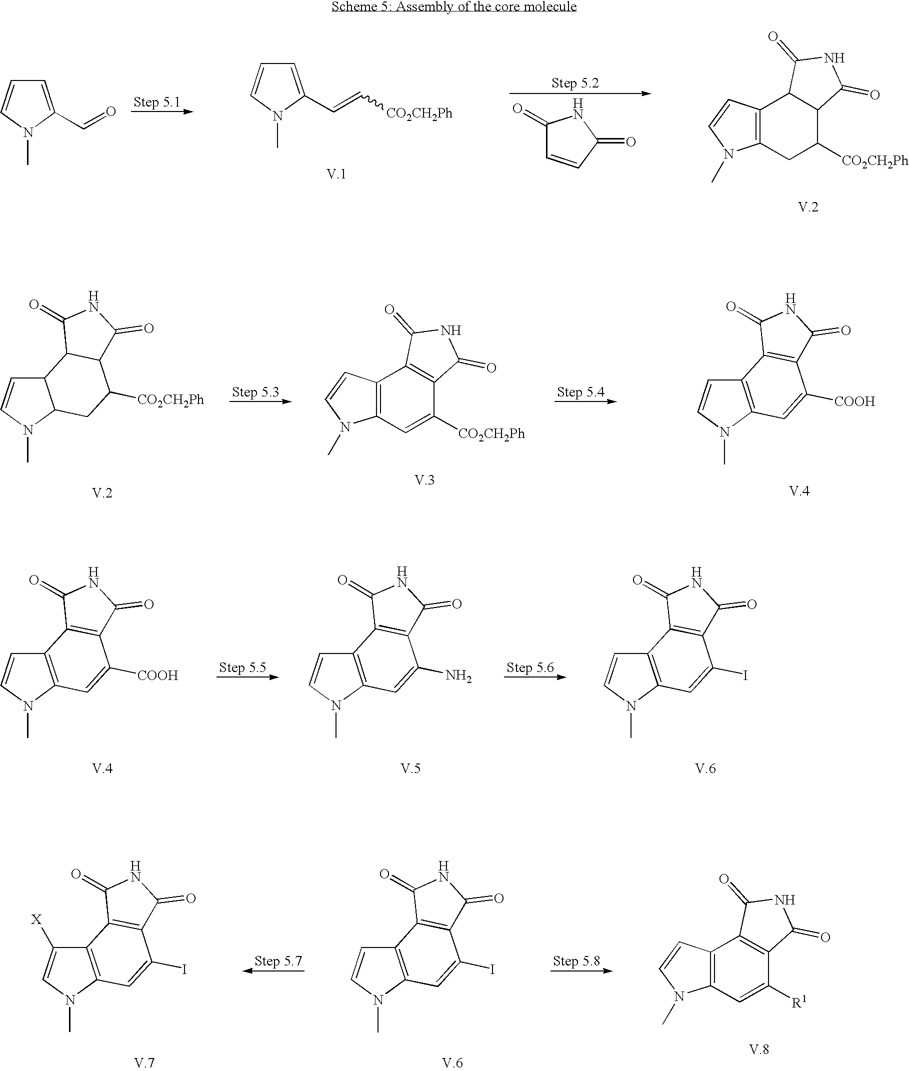

### Procedures for Scheme 5

### EXAMPLE 126

### 3-(1-Methyl-1H-pyrrol-2-yl)acrylic acid benzyl ester

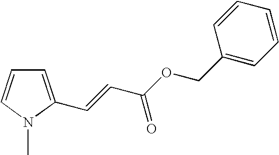

Benzyl (triphenylphosphoranylidene)acetate (7.89 g, 0.019 mol) was added to a stirred solution of 1-methylpyrrole-2-carboxaldehyde (2.00 g, 0.018 mol) in CH₂Cl₂ (200 mL) and the solution was refluxed for 6 h and cooled overnight. Silica was added and the product was adsorbed by removal of solvent in vacuo, and chromatographed directly. Elution with ethyl acetate/petroleum ether (1:4) gave the title compound as a pale yellow oil (4.06 g, 93%). ¹H NMR (400 MHz, DMSO-D6) δ ppm 3.70 (s, 3H) 5.19 (s, 2H), 6.11 (dd, J=3.7, 2.6 Hz, 1H), 6.25 (d, J=15.7 Hz, 1H), 6.79 (dd, J=3.9, 1.6 Hz, 1H), 7.00 (br, 1H), 7.42-7.30 (m, 5H), 7.58 (d, J=15.7 Hz, 1H). API-CI Found: 242 (M+1).

### EXAMPLE 127

### 6-Methyl-1,3-dioxo-1,2,3,3a,4,5,6,8b-octahydro-2,6-diaza-as-indacene-4-carboxylic acid benzyl ester

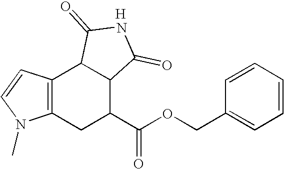

Maleimide (2.41 g, 0.025 mol) was added to 3-(1-methyl-1H-pyrrol-2-yl)acrylic acid benzyl ester (4.00 g, 0.016 mol), prepared as described in Example 126 in a 250 mL flat-bottomed flask and the mixture was warmed at 185° C. for 3 h. The solid melt was cooled to room temperature and diethyl ether (100 mL) was added. The solid mass was partially broken up with a spatula and the mixture was stirred vigorously overnight, after which time the product was present as a cream precipitate. Filtration followed by washing with diethyl ether gave material of sufficient purity for the subsequent reaction, as a mixture of diastereomers which was used directly (5.03 g, 90%), mp 189-193° C. API-CL Found: 337 (M−1).

### EXAMPLE 128

### 6-Methyl-1,3-dioxo-1,2,3,6-tetrahydro-2,6-diaza-as-indacene-4-carboxylic acid benzyl ester

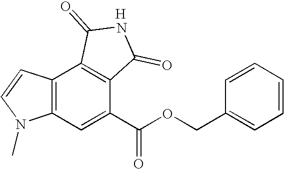

Activated manganese dioxide (20 g, 5× weight of substrate) was added to a solution of the adduct 6-methyl-1,3-dioxo-1,2,3,3a,4,5,6,8b-octahydro-2,6-diaza-as-indacene-4-carboxylic acid benzyl ester (4.00 g, 0.012 mol), prepared as described in Example 127, in p-dioxane (300 μL) and the mixture was refluxed with vigorous stirring for 2 h. The mixture was filtered hot through a plug of Celite, which was washed exhaustively with a MeOH/p-dioxane (1:1) mixture until the washings were colorless. The combined washings and filtrate were concentrated to dryness and the residue was adsorbed onto silica and chromatographed. Elution with ethyl acetate/petroleum ether (1:1) gave the title compound (1.10 g, 27%), which crystallised from ethyl acetate/petroleum ether as cream needles, mp 186-189° C. ¹H NMR (400 MHz, DMSO-D6) δ ppm 3.95 (s, 3H) 5.38 (s, 2H), 6.87 (dd, J=3.4, 0.8 Hz, 1H), 7.42-7.31 (m, 3H), 7.53-7.49 (m, 2H), 7.87 (d, J=3.4 Hz, 1H), 8.10 (d, J=0.8 Hz, 1H), 11.03 (br s, 1H). API-CI Found: 335 (M+1).

### EXAMPLE 129

### 6-Methyl-1,3-dioxo-1,2,3,6-tetrahydro-2,6-diaza-as-indacene-4-carboxylic acid

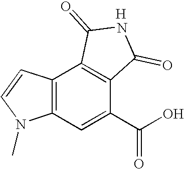

6N HCl (30 mL) was added to a solution of the 6-methyl-1,3-dioxo-1,2,3,6-tetrahydro-2,6-diaza-as-indacene-4-carboxylic acid benzyl ester (1.5 g, 4.48 mmol) in p-dioxane (150 mL) and the mixture was refluxed for 2 h. Water (1.5 L) was added and the mixture was chilled at 5° C. The product was collected by filtration, washed well with water and recrystallised from ethyl acetate/petroleum ether, to give 6-methyl-1,3-dioxo-1,2,3,6-tetrahydro-2,6-diaza-as-indacene-4-carboxylic acid as a tan powder (0.871 g, 78%), mp 313-318° C. ¹H NMR (400 MHz, DMSO-D6) δ ppm 3.96 (s, 3H) 6.87 (dd, J=3.1, 0.7 Hz, 1H), 7.89 (d, J=3.1 Hz, 1H), 8.20 (s, 1H), 11.35 (br, 1H), 13.42 (br, 1H). API-CI Found: 243 (M−1).

### EXAMPLE 130

### 4-Amino-6-methyl-6H-2,6-diaza-as-indacene-1,3-dione

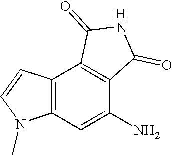

Diphenylphosphoryl azide (0.191 mL, 0.89 mmol) was added to a mixture of 6-methyl-1,3-dioxo-1,2,3,6-tetrahydro-2,6-diaza-as-indacene-4-carboxylic acid (0.20 g, 0.86 mmol), prepared as described in Example 129, and Et₃N (0.124 mL, 0.89 mmol) in anhydrous t-butanol (20 mL) (freshly distilled from sodium) and the mixture was refluxed under an atmosphere of nitrogen for 16 h. The solution was concentrated in vacuo and the residue partitioned between EtOAc and saturated aqueous NaHCO₃. Insoluble material was removed by filtration of the two layers through Celite, washing through with more EtOAc. Workup of the combined organic solutions gave the crude t-butylcarbamate as a yellow solid. This material was dissolved in CH₂Cl₂/trifluoroacetic acid (1:1) (20 mL) and the solution was stood at room temperature for 1 h. After concentration in vacuo the residue was partitioned between EtOAc and saturated aqueous NaHCO₃ solution. The EtOAc solution was worked up to give an orange solid which was adsorbed on silica and chromatographed. Elution with ethyl acetate/petroleum ether (1:1) gave the title compound as an orange powder (0.128 g, 74%), mp 281-286° C. ¹H NMR (400 MHz, DMSO-D6) δ ppm 3.67 (s, 3H) 5.96 (br s, 2H), 6.50 (dd, J=3.2, 0.8 Hz, 1H), 6.83 (d, J=0.8 Hz, 1H), 7.37 (d, J=3.2 Hz, 1H), 10.62 (br s, 1H). API-CI Found: 216 (M+1).

### EXAMPLE 131

### 4-Iodo-6-methyl-6H-2,6-diaza-as-indacene-1,3-dione

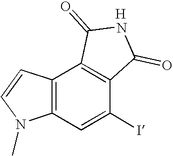

Concentrated H₂SO₄ (20 mL) was added at room temperature to powdered 4-amino-6-methyl-6H-2,6-diaza-as-indacene-1,3-dione (1.00 g, 4.97 mmol), prepared as described in Example BP 130 and the mixture was stirred for 5 min and then cooled in an ice bath. An ice-water slurry (ca 100 mL) was added in one portion with vigorous stirring and the mixture was stirred for a further 15 min to give a tan precipitate of the hydrogensulfate salt of the amine. When the internal temperature had reached 3° C. a solution of NaNO₂ (0.51 g, 7.45 mmol) in cold water (5 mL) was added dropwise over 30 seconds and the mixture was stirred for an additional 3 minutes. Powdered urea (0.21 g, 3.45 mmol) was added and the mixture was stirred for another 3 min. Finally a suspension of KI (4.12 g, 0.025 mol) and CuI (4.76 g, 0.025 mol) in cold water (80 mL) was added and the mixture was stirred well for 5 min, then the cooling bath was removed and the suspension was warmed slowly to 70° C. and held at this temperature for 1 h. 2N aqueous sodium metabisulfite solution (50 mL) was added and the mixture stirred vigorously for 5 min. Ethyl acetate was added and the two-phase mixture was filtered through a plug of Celite, washing through with more EtOAc. The combined organic portions were worked up to give an oil which was chromatographed on silica. Elution with EtOAc gave the title iodide which was crystallised from ethyl acetate/petroleum ether as a yellow powder (0.48 g, 31%), mp 322-325° C. ¹H NMR (400 MHz, DMSO-D6) δ ppm 3.88 (s, 3H) 6.78 (dd, J=3.1, 0.8 Hz, 1H), 7.70 (d, J=3.1 Hz, 1H), 8.28 (d, J=0.8 Hz, 1H), 11.06 (br s, 1H). Elemental Analysis for C₁₁H₇₁N₂O₂.¼EtOAc % C (calc/found) 41.40/41.38, % H 2.61/2.12, % N 8.05/8.26.

### EXAMPLE 132

### 8-Bromo-4-iodo-6-methyl-6H-2,6-diaza-as-indacene-1,3-dione

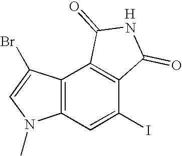

Bromine (13.6 μL, 0.26 mmol) was added dropwise at −60° C. to a solution of 4-iodo-6-methyl-6H-2,6-diaza-as-indacene-1,3-dione (0.08 g, 0.25 mmol), prepared as described in Example 131 in CHCl₃ (100 mL) and the solution was stirred at this temperature for a further 5 min. 1N aqueous sodium sulfite solution (10 mL) was added, the cooling bath was removed, and the mixture was stirred vigorously while warming to room temperature. The aqueous phase was removed and the organic layer was worked up and chromatographed on silica. Elution with ethyl acetate/petroleum ether (2:3) gave the title bromide (0.07 g, 69%), which crystallized from ethyl acetate/petroleum ether as a yellow powder, mp 242-242° C. (dec). ¹H NMR (400 MHz, DMSO-D6) δ ppm 3.86 (s, 3H) 7.89 (s, 1H), 8.37 (s, 1H), 11.14 (br s, 1H). FABMS found: [M+H]+404.8753, 406.8694. C₁₁H₇BrIN₂O₂ requires 404.8736, 406.8715.

### EXAMPLE 133

### 6-Methyl-4-(2,6-dimethoxyphenyl)-6H-2,6-diaza-as-indacene-1,3-dione

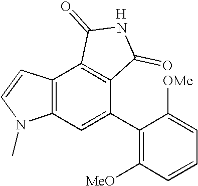

A mixture of 4-iodo-6-methyl-6H-2,6-diaza-as-indacene-1,3-dione (0.128 g, 0.39 mmol), prepared as described in Example 131 and 2,6-dimethoxybenzeneboronic acid (85 mg, 0.47 mmol) in p-dioxane (10 mL) and 2N Na₂CO₃ (2.0 mL) was purged with nitrogen. Pd(dppf)Cl₂ (32 mg, 0.039 mmol) was added and the mixture was refluxed under N₂ for 6 h and then partitioned between EtOAc and water. The organic layer was worked up, adsorbed onto silica and chromatographed. Elution with EtOAc/petroleum ether (2:3) gave the title compound (0.082 g, 62%) which crystallised from EtOAc/petroleum ether as a yellow powder, mp 285-288° C. ¹H NMR (400 MHz, DMSO-D6) δ ppm 3.62 (s, 6H), 3.85 (s, 3H), 6.74 (d, J=8.4 Hz, 2H), 6.79 (dd, J=3.1, 0.8 Hz, 1H), 7.33 (t, J=8.4 Hz, 1H), 7.54 (d, J=0.8 HZ, 1H), 7.69 (d, J=3.1 Hz, 1H), 10.64 (br s, 1H). Elemental Analysis for C₁₉H₁₆N_{204.1/4}H₂O, % C (calc/found) 66.95/67.04, % H 4.88/4.77, % N 8.22/8.10.

### EXAMPLE 134

### 6-Methyl-4-(2-methylsulfanylphenyl)-6H-2,6-diaza-as-indacene-1,3-dione (BP V.8.2) and 4-(2-Methanesulfinylphenyl)-6-methyl-6H-2,6-diaza-as-indacene-1,3-dione

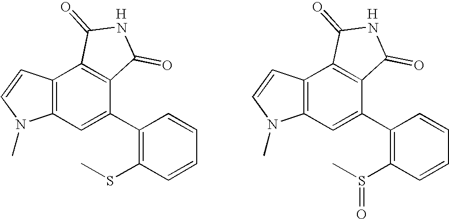

Reaction of 4-iodo-6-methyl-6H-2,6-diaza-as-indacene-1,3-dione, prepared as described in Example 131, with 2-(methylsulfanyl)benzeneboronic acid as described in Example 133 gave an oil which was chromatographed on silica. Elution with ethyl acetate/petroleum ether (1:1) gave the 6-methyl-4-(2-methylsulfanylphenyl)-6H-2,6-diaza-as-indacene-1,3-dione (54%) as a yellow solid, mp 272-274° C. ¹H NMR (400 MHz, DMSO-D6) 8 ppm 2.31 (s, 3H), 3.88 (s, 3H), 6.83 (dd, J=3.1, 0.8 Hz, 1H), 7.21-7.26 (m, 2H), 7.39-7.44 (m, 2H), 7.62 (d, J=0.8 Hz, 1H), 7.75 (d, J=3.1 Hz, 1H), 10.76 (br s, 1H). Elemental Analysis for C₁₈H₁₄N₂SO₂.½H₂O, % C (calc/found) 65.24/65.47, % H 4.56/4.38, % N 8.45/8.30.

Elution with EtOAc gave 4-(2-methanesulfinylphenyl)-6-methyl-6H-2,6-diaza-as-indacene-1,3-dione (18%) as a yellow solid, mp 311-315° C. ¹H NMR (400 MHz, DMSO-D6) δ ppm 2.32, 2.42 (2s, 3H), 3.90 (s, 3H), 6.86 (dd, J=3.1, 0.8 Hz, 1H), 7.40 (br d, J=7.4 Hz), 7.58-7.66 (m), 7.69-7.75 (m), 7.78-7.84 (m), 7.94-8.04 (m), 10.90 (br s, 1H). FABMS found: [M+H]+339.0800. C₁₈H₁₅N₂SO₃ requires 339.0803.

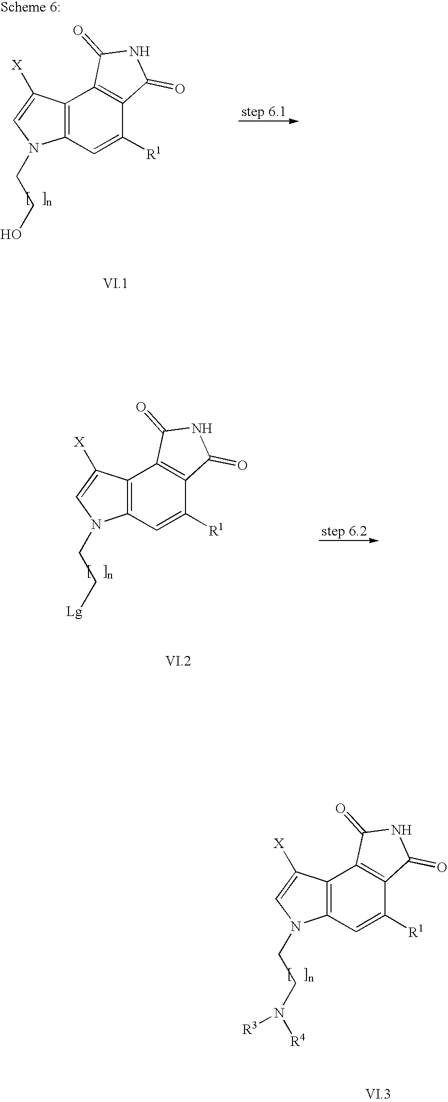

### Procedures for Scheme 6

### EXAMPLE 135

### 8-Bromo-4-(2-chlorophenyl)-6-[3-(cis-3,5-dimethylpiperazin-1-yl)propyl]-6H-2,6-diaza-as-indacene-1,3-dione

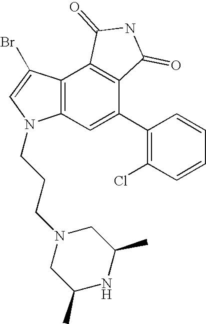

A solution of the bromide 8-bromo-6-(3-bromopropyl)-4-(2-chlorophenyl)-6H-2,6-diaza-as-indacene-1,3-dione, prepared as described in Example 35 (75 mg, 0.15 mmol) and cis-2,6-dimethylpiperazine (35 mg, 0.30 mmol) in dimethylacetamide (7 mL) was stirred at room temperature for 16 h. After dilution with saturated aqueous NaHCO₃ solution the mixture was extracted with ethyl acetate and worked up to give the title amine (53.3 mg, 67%). ¹H NMR (400 MHz, DMSO-D6) δ ppm 0.79 (d, J=6.2 Hz, 3H), 0.83 (d, J=6.2 Hz, 3H), 1.24-1.33 (m, 2H), 1.82-1.97 (m, 2H), 2.09 (t, J=6.4 Hz, 2H), 2.40-2.58 (m), 4.25-4.37 (m, 2H), 7.38-7.47 (m, 3H), 7.52-7.55 (m, 1H), 7.85 (s, 1H), 8.01 (s, 1H), 10.89 (br, 1H). The hydrochloride salt crystallised from methanol/diethyl ether as a yellow powder, mp 236-239° C. API-CI Found: 529, 531 (M+1). Elemental Analysis for C₂₅H₂₆BrClN₄O₂.2.5H₂O % C (calc/found) 52.23/52.04, % H 5.43/5.03, % N 9.74/9.76.sd

### EXAMPLE 136

### 6-(3-Aminopropyl)-8-bromo-4-(2-chlorophenyl)-6H-2,6-diaza-as-indacene-1,3-dione; hydrochloride

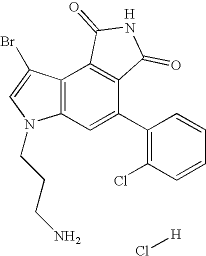

Anhydrous ammonia gas was slowly bubbled through a solution of 8-bromo-6-(3-bromopropyl)-4-(2-chlorophenyl)-6H-2,6-diaza-as-indacene-1,3-dione, prepared as described in Example 35 (114 mg, 0.23 mmol) in 2-propanol (200 mL) at 20° C. for 2 h. The reaction flask was sealed and stirred for a month. It was partitioned in between ethyl acetate and cold aq. sodium carbonate solution. The ethyl acetate solution was further washed water (3×), dried (Na₂SO₄), evaporated and columned (silica gel; chloroform/methanol=10:1) to give the free base of 6-(3-aminopropyl)-8-bromo-4-(2-chlorophenyl)-6H-2,6-diaza-as-indacene-1,3-dione (70 mg, 70%). It was converted into its hydrochloride salt to give a yellow solid; mp 310-313° C.; API-CI Found: 431.8 (M+1, 70%), 432.8 (10), 433.8 (100), 435.8 (25); 429.8 (M−1, 75%), 431.8 (100), 433.8 (20); ¹H NMR (400 MHz, DMSO-D6) δ ppm 2.04 (m, 2H) 2.77 (m, 2H) 4.41 (m, 2H) 7.41-7.57 (m, 4H) 7.84 (br s, exchangeable with D₂O, 3H) 7.92 (s, 1H) 8.08 (s, 1H) 10.70 (s, exchangeable with D₂O, 1H); FABMS found [M+H]⁺: 432.0091, 434.0072, 436.0091. C₁₉H₁₆BrClN₃O₂ requires 432.0114, 434.0084 (434.0093), 436.0064.

### EXAMPLE 137

### 8-Bromo-4-(2-chlorophenyl)-6-(3-pyrrolidin-1-ylpropyl)-6H-2,6-diaza-as-indacene-1,3-dione

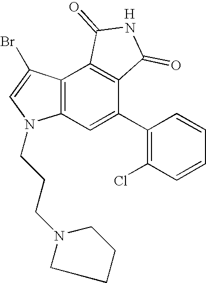

A solution of 8-bromo-4-(2-chlorophenyl)-6-(3-hydroxypropyl)-6H-2,6-diaza-as-indacene-1,3-dione, prepared as described in Example 45 (103 mg, 0.237 mmol) in dichloromethane (10 mL) was treated with methanesulfonyl chloride (0.2 mL) in the presence of triethylamine (1 mL) at 0° C. for 30 min. It was diluted with dichloromethane and washed successively with cold aq. sodium carbonate and water. The organic solution was dried (Na₂SO₄) and evaporated to give a residue which was treated with an excess of sodium iodide in ethyl acetate at reflux for 1 h. It was diluted with ethyl acetate and washed successively with aq. Na₂S₂O₅ and water. The ethyl acetate solution was dried (Na₂SO₄) and evaporated to give an orange solid which was treated with pyrrolidine (0.05 mL, 0.284 mmol) and triethylamine (0.1 mL) in DMF (2 mL) at 20° C. for 24 h. The solution was partitioned between dichloromethane and cold aq. sodium carbonate solution. Evaporation of solvent gave an orange residue which was treated with ammonium acetate at 150° C. for 1 h. Dichloromethane added to the cooled melt and after basification with sodium carbonate at 0° C., the organic layer was separated, washed with water, and evaporated to give crude product. Chromatography on a silica column (chloroform/methanol=20:1) gave pure 8-bromo-4-(2-chlorophenyl)-6-(3-pyrrolidin-1-ylpropyl)-6H-2,6-diaza-as-indacene-1,3-dione (67 mg, 58%); mp 158-161° C.; ¹H NMR (400 MHz, DMSO-D6) δ ppm 1.55 (m, 4H) 1.90 (m, 2H) 2.22-2.35 (m, 6H) 4.27-4.38 (m, 2H) 7.39-7.48 (m, 3H) 7.52-7.56 (m, 1H) 7.88 (s, 1H) 8.01 (s, 1H) 10.91 (s, 1H); FABMS found [M+H]⁺: 486.0566, 488.0548, 490.0544. C₂₃H₂₂BrClN₃O₂ requires 486.0583, 488.0554 (488.0563), 490.0533.

### EXAMPLE 138

### 8-Bromo-4-(2-chlorophenyl)-6-[3-(4-methylpiperazin-1-yl)-propyl]-6H-2,6-diaza-as-indacene-1,3-dione

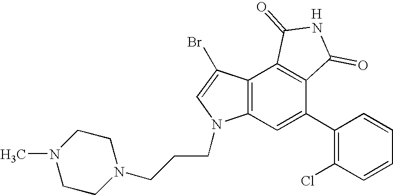

Using the method of Example 137, the title compound was prepared from 8-bromo-4-(2-chlorophenyl)-6-(3-hydroxypropyl)-6H-2,6-diaza-as-indacene-1,3-dione (87 mg, 0.20 mmol), N-methylpiperazine (60 mg, 0.60 mmol) and triethylamine (0.10 mL) in 11% yield after purification on a silica column (chloroform/methanol=10:1); mp 150-152° C.; API-CL Found: 515.0 (M+1, 80%), 517.0 (100%), 519.0 (25); 512.8 (M−1, 75%), 514.8 (100), 516.8 (25); ¹H NMR (400 MHz, DMSO-D6) δ ppm 1.89 (m, 2H) 2.01 (s, 3H) 2.05-2.29 (m, 10H) 4.30 (m, 2H) 7.40-7.48 (m, 3H) 7.51-7.56 (m, 1H) 7.87 (s, 1H) 8.01 (s, 1H) 10.90 (s, 1H); FABMS found [M+H]⁺: 515.0835, 517.0819, 519.0789. C₂₄H₂₅BrClN₄O₂ requires 515.0849, 517.0819 (517.0828), 519.0799.

### EXAMPLE 139

### 8-Bromo-4-(2-chlorophenyl)-6-(3-morpholin-4-ylpropyl)-6H-2,6-diaza-as-indacene-1,3-dione

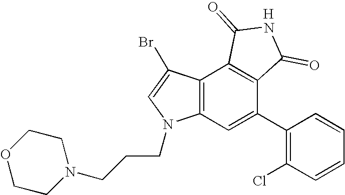

Using the method of Example 137, except that 2-propanol (5 mL) was used in place of DMF with a reaction time 70 h, the title compound was prepared from 8-bromo-4-(2-chlorophenyl)-6-(3-hydroxypropyl)-6H-2,6-diaza-as-indacene-1,3-dione (87 mg, 0.20 mmol) and morpholine (0.10 mL) in 70% yield after purification by a silica column (chloroform/methanol=10:1); mp 117-120° C.; API-CI Found: 501.8 (M+1, 75%), 503.8 (100%), 505.8 (25); 499.8 (M−1, 75%), 501.8 (100), 503.8 (25); ¹H NMR (400 MHz, DMSO-D6) δ ppm 1.90 (m, 2H) 2.10-2.28 (m, 6H) 3.30-3.55 (m, 4H) 4.33 (m, 2H) 7.40-7.48 (m, 3H) 7.52-7.56 (m, 1H) 7.88 (s, 1H) 8.03 (s, 1H) 10.91 (s, 1H); FABMS found [M+H]⁺: 502.0497, 504.0506, 506.0499. C₂₃H₂₂BrClN₃O₃ requires 502.0533, 504.0503 (504.0512), 506.0483.

### EXAMPLE 140

### 8-Bromo-4-(2-chlorophenyl)-6-(3-dimethylaminopropyl)-6H-2,6-diaza-as-indacene-1,3-dione

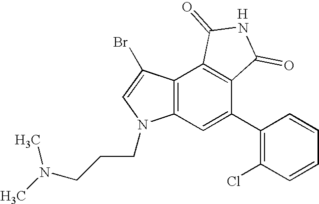

Using the method of Example 137, except that 2-propanol (5 mL) was used in place of DMF with a reaction time 46 h, the title compound was prepared from 8-bromo-4-(2-chlorophenyl)-6-(3-hydroxypropyl)-6H-2,6-diaza-as-indacene-1,3-dione (87 mg, 0.20 mmol) and aq. 40% dimethylamine (0.20 mL) in 58% yield after purification by a silica column (chloroform/methanol=20:1); mp 160-162° C.; API-CI Found: 459.8 (M+1, 85%), 462.0 (100%), 463.8 (25); 457.8 (M−1, 85%), 459.8 (100), 461.8 (25); ¹H NMR (400 MHz, DMSO-D6) δ ppm 1.88 (m, 2H) 2.06 (s, 6H) 2.11 (t, J=6.7 Hz, 2H) 4.31 (t, J=6.7 Hz 2H) 7.39-7.48 (m, 3H) 7.52-7.56 (m, 1H) 7.84 (s, 1H) 8.01 (s, 1H) 10.91 (s, 1H); FABMS found [M+H]⁺: 460.0418, 462.0405, 464.0392. C₂₁H₂₀BrClN₃O₂ requires 460.0427, 462.0406 (462.0397), 464.0377.

### EXAMPLE 141

### 8-Bromo-4-(2-chlorophenyl)-6-(3-dipropylaminopropyl)-6H-2,6-diaza-as-indacene-1,3-dione

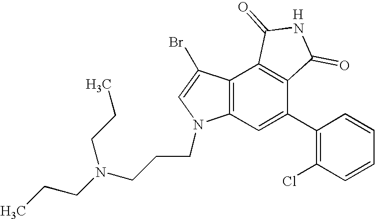

Using the method of Example 137, except that 2-propanol (5 mL) was used in place of DMF with a reaction time 70 h, the title compound was prepared from 8-bromo-4-(2-chlorophenyl)-6-(3-hydroxypropyl)-6H-2,6-diaza-as-indacene-1,3-dione (87 mg, 0.20 mmol) and di-n-propylamine (0.20 mL) in 61% yield after purification by a silica column (chloroform/methanol=20:1); mp 173-175° C.; API-CI Found: 516.0 (M+1, 80%), 518.0 (100%), 519.0 (25); 514.0 (M−1, 65%), 515.8 (100), 517.0 (25); ¹H NMR (400 MHz, DMSO-D6) δ ppm 0.76 (t, J=7.4 Hz, 6H) 1.29 (m, 4H) 1.86 (m, 2H) 2.24 (m, 4H) 2.31 (t, J=6.8 Hz, 2H) 4.30 (m, 2H) 7.39-7.47 (m, 3H) 7.52-7.56 (m, 1H) 7.81 (s, 1H) 8.01 (s, 1H) 10.92 (s, 1H); FABMS found [M+H]⁺: 516.1048, 518.1026, 520.1012. C₂₅H₂₈BrClN₃O₂ requires 516.1053, 518.1023 (518.1033), 520.1003.

### EXAMPLE 142

### 4-(2-Chlorophenyl)-6-(2-pyrrolidin-1-ylethyl)-6H-2,6-diaza-as-indacene-1,3-dione

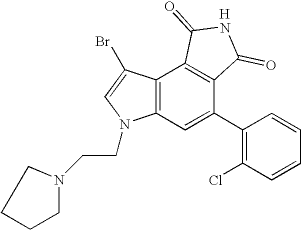

Using the method of Example 137, except that DMA was used in place of DMF, with a reaction time of 2 h at 80-90° C., the title compound was prepared from 4-(2-chlorophenyl)-6-(2-hydroxyethyl)-6H-pyrrolo[3,4-e]indole-1,3-dione (84 mg, 0.247 mmol) and pyrrolidine (0.20 mL) in 18% yield after purification by a silica column (chloroform/methanol=10:1); mp 208-209° C.; ¹H NMR (400 MHz, DMSO-D6) δ ppm 1.62 (m, 4H) 2.44 (m, 4H) 2.79 (t, J=6.3 Hz, 2H) 4.40 (t, J=6.3 Hz, 2H) 6.85 (dd, J=3.1 & 0.6 Hz, 1H) 7.38-7.48 (m, 3H) 7.52-7.57 (m, 1H) 7.74 (d, J=0.6 Hz, 1H) 7.83 (d, J=3.1 Hz, 1H) 10.81 (s, 1H); FABMS found [M+H]⁺: 394.1309, 396.1303. C₂₂H₂₁ClN₃O₂ requires 394.1322, 396.1292.

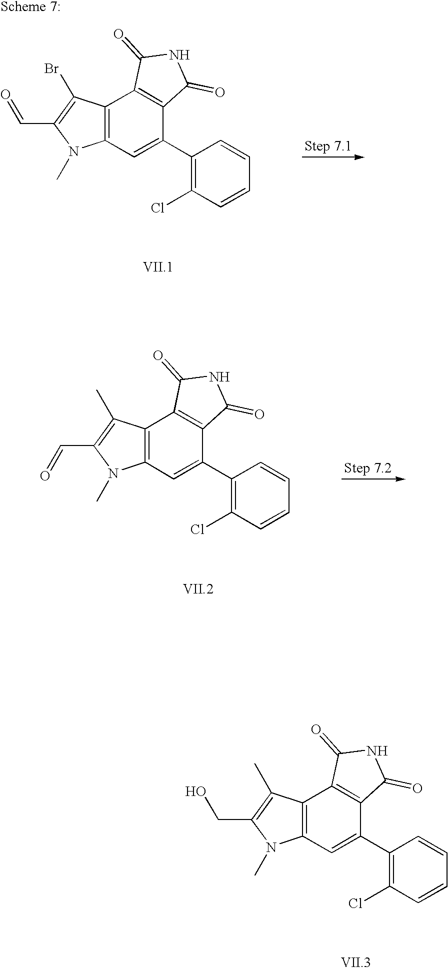

### Procedures for Scheme 7

### EXAMPLE 143

### 4-(2-Chlorophenyl)-6,8-dimethyl-1,3-dioxo-1,2,3,6-tetrahydro-2,6-diaza-as-indacene-7-carbaldehyde

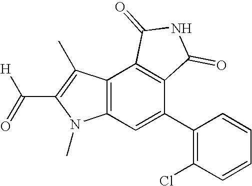

8-Bromo-4-(2-chlorophenyl)-6-methyl-1,3-dioxo-1,2,3,6-tetrahydro-2,6-diaza-as-indacene-7-carbaldehydehyde, prepared as described in Example 73 (0.500 g, 1.20 mmol), cesium carbonate (1.20 g, 3.60 mmol) and tetrakis(triphenylphosphino) palladium (0) were combined in a flask and the flask was purged with argon. Dioxane (25 mL) was added and the flask purged with argon again. To the reaction mixture trimethoxyboroxine (0.173 g, 1.38 mmol) was added and the reaction was heated to reflux for 1 h. The reaction mixture was cooled and stirred for an additional hour before pouring into water (100 mL) and extracting four times with ethyl acetate (50 mL each). The organic phases were combined and washed twice with brine (50 mL each), dried over MgSO₄ and concentrated. The crude product was purified by flash chromatography using a 10 g ISCO silica cartridge eluted with ethyl acetate/hexane gradient to give 255 mg of the title compound as a yellow solid; ¹H NM (400 MHz, CDCl₃) δ ppm 3.04 (s, 3H) 4.11 (s, 3H) 7.38 (m, 4H) 7.48 (s, 1H) 7.51 (m, 1H) 10.32 (s, 1H); Elemental Analysis for C₁₉H₁₃ClN₂O₃.0.20C₄H₈O₂% C (calc/found) 63.97/64.07, % H 4.00/3.67, % N 7.61/7.25.

### EXAMPLE 144

### 4-(2-Chlorophenyl)-7-hydroxymethyl-6,8-dimethyl-6H-2,6-diaza-as-indacene-1,3-dione

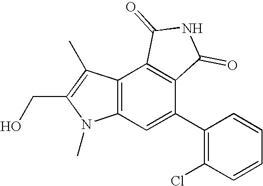

The title compound was prepared from 4-(2-chlorophenyl)-6,8-dimethyl-1,3-dioxo-1,2,3,6-tetrahydro-2,6-diaza-as-indacene-7-carbaldehyde, prepared as described in Example 143 (0.104 g, 0.29 mmol), using the procedure described in Example 75 to give 0.040 g of the title compound as a yellow solid; ¹H NMR (400 MHz, DMSO-D6) δ ppm 2.60 (s, 3H) 3.82 (s, 3H) 4.67 (s, 2H) 5.27 (s, 1H) 7.43 (m, 3H) 7.52 (dt, J=5.86, 1.95 Hz, 1H) 7.63 (s, 1H) 10.84 (s, 1H); LCMS (Phenomenex Develosil Combi RP3 50×4.6 mm column, gradient method 70-2% acetonitrile/water eluent (0.1% formic acid) in 2.0 min with 2 min hold, MSD in ESI positive mode) 90% at 2.89 min (UV_{254 nm}); API-ES 124.1 (100), 337.0 (40), 357.0 (10).

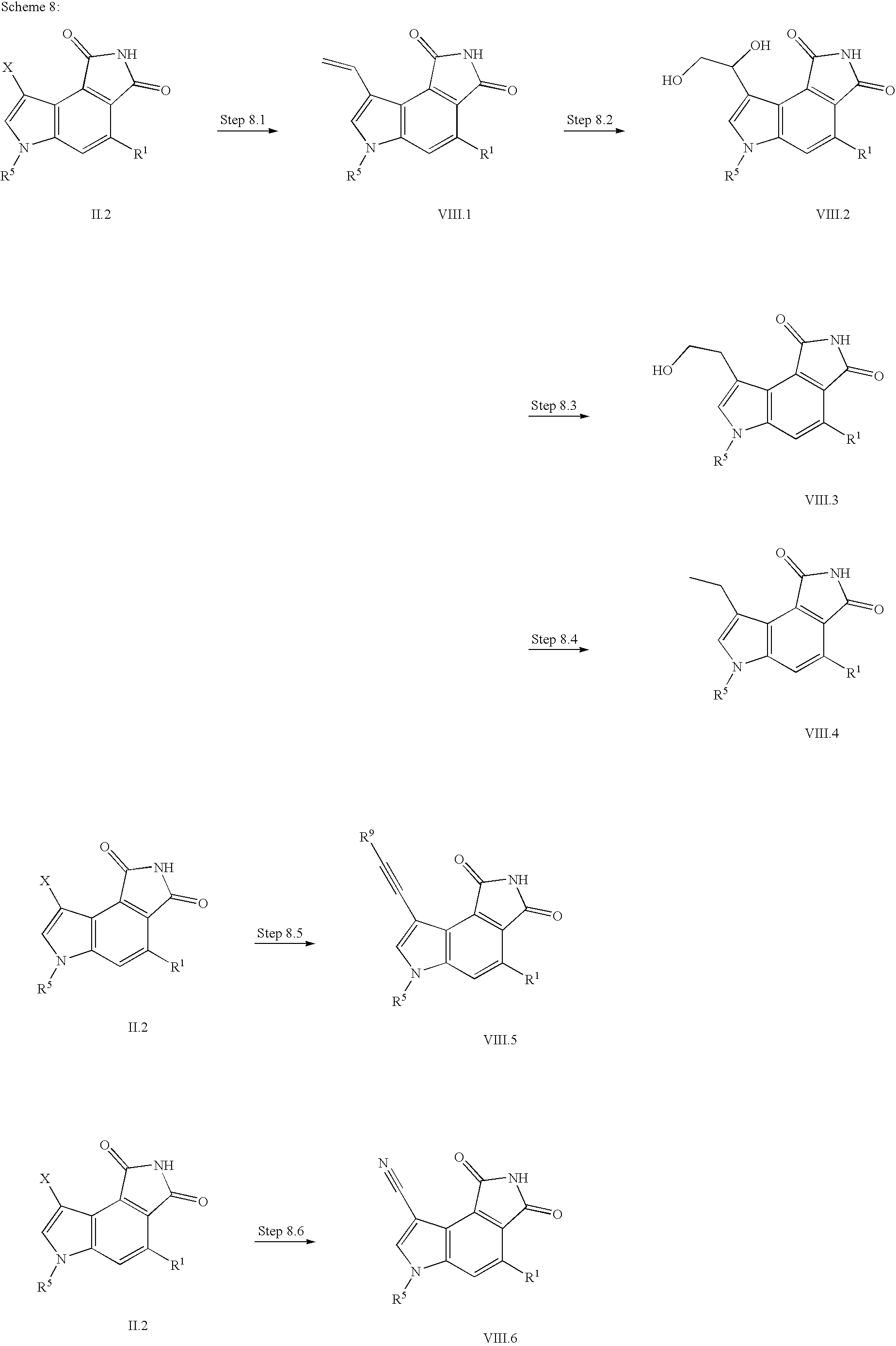

### Procedures for Scheme 8

### EXAMPLE 145

### 4-(2-Chlorophenyl)-6-methyl-8-vinyl-6H-2,6-diaza-as-indacene-1,3-dione

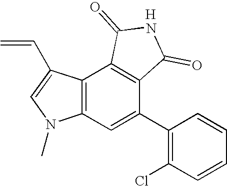

Pd(PPh₃)₄ (0.16 g, 0.14 mmol) was added to a nitrogen-purged suspension of bromide 8-bromo-4-(2-chlorophenyl)-6-methyl-6H-2,6-diaza-as-indacene-1,3-dione, prepared as described in Example 37 (0.27 g, 0.69 mmol) and tri-n-buytlvinyltin (0.30 mL) in toluene (80 mL) and the mixture was refluxed under nitrogen for 18 h. The cooled solution was diluted with ethyl acetate and washed with saturated aqueous NaHCO₃, water and brine. After drying over Na₂SO₄ the product was adsorbed onto silica by concentration in vacuo, and chromatographed. Elution with ethyl acetate/petroleum ether (2:3) gave the alkene (BP VIII.1.1) (0.142 g, 61%), which crystallised from tetrahydrofuran/petroleum ether as an orange powder, mp 200-205° C. ¹H NMR (400 MHz, DMSO-D6) δ ppm 3.90 (s, 3H), 5.11 (dd, J=11.1, 1.7 Hz, 1H), 5.60 (dd, J=17.7, 1.7 Hz, 1H), 7.39-7.47 (m, 3H), 7.52-7.56 (m, 1H), 7.70 (s, 1H), 7.84 (dd, J=17.7, 11.1 Hz, 1H), 8.13 (s, 1H), 10.92 (br s, 1H). API-CI Found: 337, 339 (M+1). Elemental Analysis for C₁₉H₁₃ClN₂O₂.¼H₂O % C (calc/found) 66.87/66.89, % H 3.99/3.85, % N 8.21/8.40.

### EXAMPLE 146

### 4-(2-Chlorophenyl)-8-(1,2-dihydroxyethyl)-6-methyl-6H-2,6-diaza-as-indacene-1,3-dione

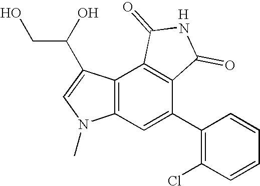

A solution of 4-(2-chlorophenyl)-6-methyl-8-vinyl-6H-2,6-diaza-as-indacene-1,3-dione (51 mg, 0.15 mmol), OsO₄ (0.1 mL of a 4% solution in water, 0.015 mmol) and N-methylmorpholine-N-oxide (35 mg, 0.30 mmol) in 4:1 acetone/water (20 mL) was stirred at room temperature for 3 h. The solution was diluted with ethyl acetate, washed well with 1N HCl, then water, and worked up to give a solid which was chromatographed on silica. Elution with ethyl acetate/petroleum ether (1:1) gave foreruns, while methanol/tetrahydrofuran/ethyl acetate (5:10:85) gave the title compound (46 mg, 82%) which crystallised from tetrahydrofuran/petroleum ether as a yellow powder, mp 179-183° C. ¹H NMR (400 MHz, DMSO-D6) δ ppm 3.42-3.52 (m, 1H), 3.64-3.71 (m, 1H), 3.86 (s, 3H), 4.51 (dd, J=5.3, 5.4 Hz, 1H), 5.04 (dd, J=5.1, 3.4 Hz, 1H), 5.47-5.52 (dd, J=10.8, 5.0 Hz, 1H), 7.39-7.47 (m, 3H), 7.52-7.56 (m, 1H), 7.65 (s, 1H), 7.68 (s, 1H), 10.90 (br s, 1H). FABMS Found [M+H]+371.0798, 373.0779. C₁₉H₁₆ClN₂O₄ requires 371.0799, 373.0769.

### EXAMPLE 147

### 4-(2-Chlorophenyl)-8-(2-hydroxyethyl)-6-methyl-6H-2,6-diaza-as-indacene-1,3-dione

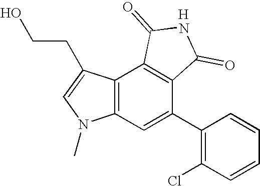

A solution of 9-BBN in tetrahydrofuran (0.43 mL of a 0.5N solution, 0.21 mmol) was added dropwise under nitrogen to a solution of the 4-(2-chlorophenyl)-6-methyl-8-vinyl-6H-2,6-diaza-as-indacene-1,3-dione (61 mg, 0.18 mmol) in dry THF (5 mL) and the solution was stirred at room temperature for 2 h. Another portion of the 9-BBN solution (0.43 mL) was added and stirring was continued for a further 2 h. Methanol (5 mL) was added, followed by 3N aqueous sodium acetate (2 mL) and 30% H₂O₂ (2 mL). After stirring for 2 h the mixture was diluted with water, extracted with ethyl acetate and worked up to give an oil which was chromatographed on silica. Elution with ethyl acetate/petroleum ether (1:1) gave the title compound (23.1 mg, 36%), which crystallised from ethyl acetate/petroleum ether as an orange powder, mp 225-229° C. ¹H NMR (400 MHz, DMSO-D6) δ ppm 3.20-3.25 (m, 2H), 3.66 (br dd, J=12.0, 6.7 Hz, 2H), 3.83 (s, 3H), 4.49 (t, J=5.2 Hz, 1H), 7.39-7.47 (m, 3H), 7.52-7.56 (m, 1H), 7.55 (s, 1H), 7.61 (s, 1H), 10.81 (br s, 1H). FABMS Found: [M+H]+355.0828, 357.0840. C₁₉H₁₆ClN₂O₃ requires 355.0849, 357.0820.

### EXAMPLE 148

### 4-(2-Chlorophenyl)-8-ethyl-6-methyl-6H-2,6-diaza-as-indacene-1,3-dione

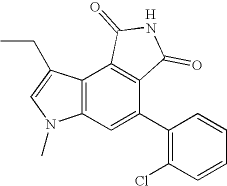

A solution of 4-(2-chlorophenyl)-6-methyl-8-vinyl-6H-2,6-diaza-as-indacene-1,3-dione (20 mg) in ethyl acetate/methanol (1:1) (10 mL) was hydrogenated over PtO₂ (10 mg) at 60 psi for 15 min. The catalyst was removed by filtration and the filtrate was adsorbed onto silica by concentration in vacuo, and chromatographed. Elution with ethyl acetate/petroleum ether (1:4) gave the product title compound (15 mg, 75%), which crystallised from ethyl acetate/petroleum ether as a pale yellow solid, mp 230-233° C. ¹H NMR (400 MHz, DMSO-D6) δ ppm 1.25 (t, J=7.4 Hz, 3H), 3.07 (q, J=7.4 Hz, 2H), 3.83 (s, 3H), 7.39-7.47 (m, 3H), 7.52-7.56 (m, 1H), 7.53 (s, 1H), 7.61 (s, 1H), 10.80 (br, 1H). EIMS Found: M+338.0819, 340.0798. C₁₉H₁₅ClN₂O₂ requires 338.0822, 340.0793.

### EXAMPLE 149

### 4-(2-Chlorophenyl)-8-ethynyl-6-methyl-6H-2,6-diaza-as-indacene-1,3-dione

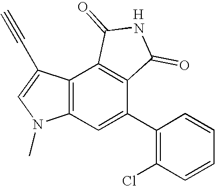

A mixture of the bromide 8-bromo-4-(2-chlorophenyl)-6-methyl-6H-2,6-diaza-as-indacene-1,3-dione, prepared as described in Example 37 (0.127 g, 0.33 mmol), trimethylsilylacetylene (0.069 mL, 0.40 mmol), copper(I) iodide (9 mg, 0.049 mmol) and butylamine (0.32 mL, 3.27 mmol) in tetrahydrofuran (6 mL) was purged with nitrogen. Pd(PPh₃)₄ (19 mg, 0.016 mmol) was added and the mixture was refluxed under nitrogen for 7 h and cooled overnight. Ethyl acetate was added and the solution was washed with water and worked up to give an oil, which was chromatographed on silica. Elution with ethyl acetate/petroleum ether (1:9) gave the trimethylsilyl derivative of the product, which was immediately dissolved in methanol (10 mL). Solid K₂CO₃ (0.10 g) was added and the mixture was stirred at room temperature for 4 h. After dilution with water the mixture was extracted with ethyl acetate and worked up to give a solid, which was chromatographed on silica. Elution with ethyl acetate/petroleum ether gave 4-(2-chlorophenyl)-8-ethynyl-6-methyl-6H-2,6-diaza-as-indacene-1,3-dione (23 mg, 21%), which crystallised from tetrahydrofuran/petroleum ether as a yellow powder, mp 125-129° C. ¹H NMR (400 MHz, DMSO-D6) δ ppm 3.88 (s, 3H), 4.00 (s, 1H), 7.39-7.48 (m, 3H), 7.52-7.56 (m, 1H), 7.76 (s, 1H), 8.10 (s, 1H), 10.88 (s, 1H). FABMS Found: [M+H]+335.0587, 337.0570. C₁₉H₁₂ClN₂O₂ requires 335.0587, 337.0558.

### EXAMPLE 150

### 8-(3-Hydroxyprop-1-ynyl)-4-(2-methoxyphenyl)-6-methyl-6H-2,6-diaza-as-indacene-1,3-dione

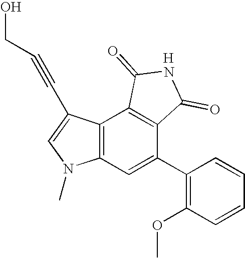

Reaction of 8-bromo-4-(2-methoxyphenyl)-6-methyl-6H-2,6-diaza-as-indacene-1,3-dione prepared as described in Example 28, with propargyl alcohol as described in Example 149 except that the treatment with K₂CO₃ was omitted gave an oil which was purified by chromatography on silica. Elution with ethyl acetate/petroleum ether (3:7) gave starting material, while ethyl acetate gave the title compound (27%), which crystallised from ethyl acetate/petroleum ether as a yellow solid, mp 154-157° C. ¹H NMR (400 MHz, DMSO-D6) δ ppm 3.65 (s, 3H), 3.87 (s, 3H), 4.36 (d, J=5.9 Hz, 2H), 5.17 (t, J=5.9 Hz, 1H), 7.02 (dt, J=7.4, 0.9 Hz, 1H), 7.07 (br d, J=7.8 Hz, 1H), 7.28 (dd, J=7.4, 1.7 Hz, 1H), 7.37-7.42 (m, 1H), 7.66 (s, 1H), 7.96 (s, 1H), 10.74 (br s, 1H). API-CI Found: 361 (M+1).

### EXAMPLE 151

### 4-(2-Chlorophenyl)-6-methyl-1,3-dioxo-1,2,3,6-tetrahydro-2,6-diaza-as-indacene-8-carbonitrile

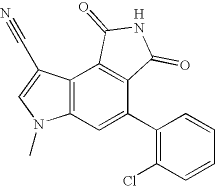

A mixture of the 4-(2-chlorophenyl)-8-iodo-6-methyl-6H-2,6-diaza-as-indacene-1,3-dione prepared as described in Example 32, (0.10 g, 0.23 mmol) and CuCN (41 mg, 0.46 mmol) in N-methylpyrrolidinone (7 mL) was refluxed for 3 h. After dilution with water the mixture was extracted into ethyl acetate and worked up to give an oil, which was chromatographed on silica. Elution with ethyl acetate/petroleum ether (15:35) gave foreruns, while ethyl acetate/petroleum ether (1:1) gave the nitrile title compound (7.8 mg, 10%), which crystallised from ethyl acetate/petroleum ether as a tan powder, mp 160-163° C. ¹H NMR (400 MHz, DMSO-D6) δ ppm 3.96 (s, 3H), 7.39-7.48 (m, 3H), 7.52-7.56 (m, 1H), 7.92 (s, 1H), 8.63 (s, 1H), 11.13 (br s, 1H). API-CI Found: 334, 336 (M−1).

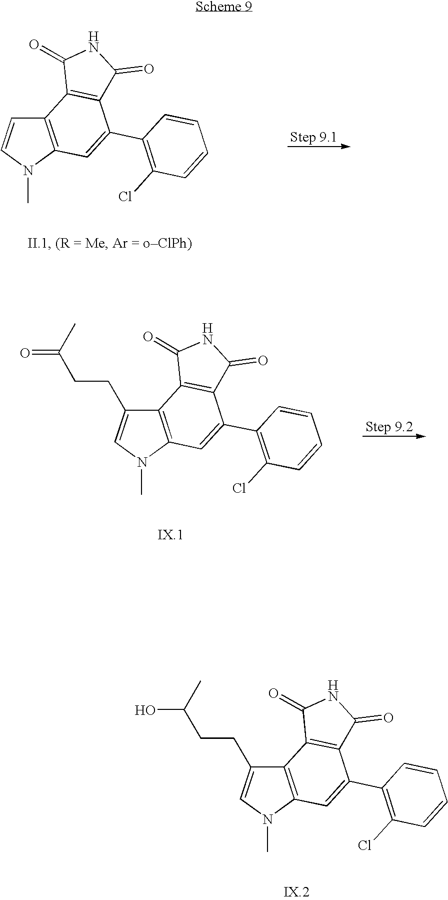

### Procedures for Scheme 9

### EXAMPLE 152

### 4-(2-Chlorophenyl)-6-methyl-8-(3-oxobutyl)-6H-2,6-diaza-as-indacene-1,3-dione

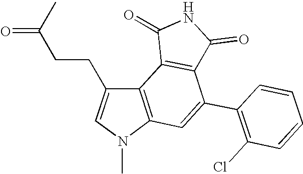

To a solution of compound 8-bromo-4-(2-chlorophenyl)-6-methyl-6H-2,6-diaza-as-indacene-1,3-dione (0.1 g) prepared as described in Example 37 in 1,2-dichloroethane (4 mL) was added indium (III) chloride (14 mg) and methyl vinyl ketone (53 ul). The reaction mixture was heated to reflux for 2 h and then a further portion of methyl vinyl ketone (53 ul) was added. After 3 h a further portion of methyl vinyl ketone (53 ul) was added, finally after a further 2 h the reaction mixture was cooled to room temperature and poured into ethyl acetate (50 mL). The organic component was washed with water (10 mL×2) and brine (10 mL×1), dried (MgSO₄) and concentrated to dryness. Purification by column chromatography using a gradient of 5% to 50% ethyl acetate in hexanes as the eluent gave the title compound (76 mg). ¹H NMR (400 MHz, DMSO-D6) δ ppm 2.12 (s, 3H), 2.80 (d, J=7.8 Hz, 2H), 3.21 (d, J=7.2 Hz, 2H), 3.82 (s, 3H) 7.43 (m, 3H), 7.52 (m, 2H), 7.63 (s, 1H), 10.88 (s, 1H). Mass spectrum APCI− 381,379.

### EXAMPLE 153

### 4-(2-Chlorophenyl)-8-(3-hydroxybutyl)-6-methyl-6H-2,6-diaza-as-indacene-1,3-dione

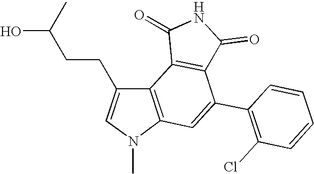

To a solution of 4-(2-chlorophenyl)-6-methyl-8-(3-oxobutyl)-6H-2,6-diaza-as-indacene-1,3-dione (0.07 g) prepared as described in Example 152, in tetrahydrofuran (5 mL) were added ethanol (2 mL), zinc chloride (70 mg) and sodium borohydride (13 mg). The reaction mixture was stirred at room temperature for 2 h and then carefully quenched with water (25 mL). The aqueous phase was extracted with ethyl acetate (3×50 mL). The combined organic phase was washed with water (10 mL), and brine (10 mL), dried and concentrated to dryness. Purification by column chromatography using a gradient of 5% to 100% ethyl acetate in hexanes as the eluent gave the title compound (65 mg); ¹H NMR (400 MHz, DMSO-D6) □ ppm 1.10 (d, J=4 Hz, 3H,) 1.68 (m, 2H), 3.05 (m, 2H), 3.69 (brs, 1H), 3.82 (s, 3H), 4.38 (d, J=4.9 Hz, 1H), 7.42 (m, 3H), 7.51 (m, 2H), 7.61 (s, 1H), 10.84 (brs, 1H). Mass Spectrum APCI− 383, 385.

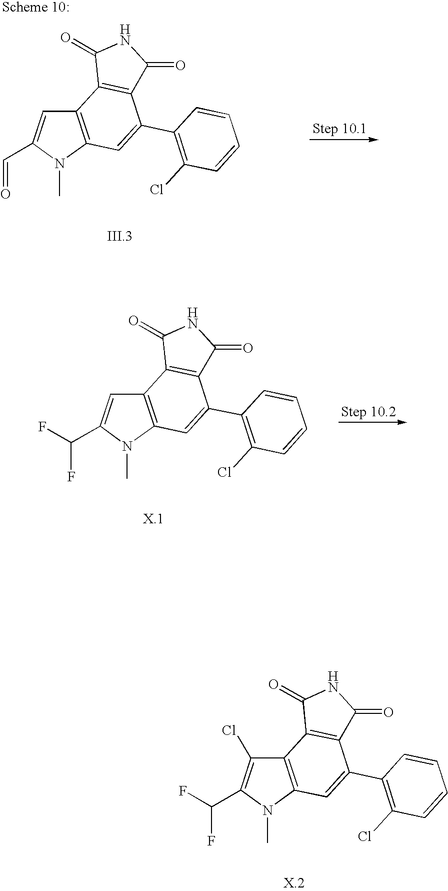

### Procedures for Scheme 10

### EXAMPLE 154

### 4-(2-Chlorophenyl)-7-difluoromethyl-6-methyl-6H-2,6-diaza-as-indacene-1,3-dione

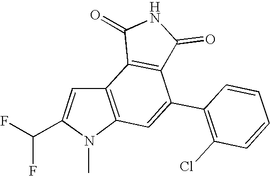

To a suspension of 4-(2-chlorophenyl)-6-methyl-1,3-dioxo-1,2,3,6-tetrahydro-2,6-diaza-as-indacene-7-carbaldehyde (0.5 g), prepared as described in Example 72, in DCE (5 mL) was added bis(2-methoxyethyl)aminosulphur trifluoride (1.0 mL). The reaction mixture was heated to 40° C. for 3 h and the cooled to room temperature. Saturated NH₄Cl (25 mL) was added and the aqueous phase was extracted with ethyl acetate (3×25 mL), dried (MgSO₄) and concentrated to dryness. Purification by column chromatography using an eluent of 50% hexane/DCM to DCM gave the title compound (0.14 g); ¹H NMR (400 MHz, DMSO-D6) δ ppm 3.94 (s, 3H) 7.22 (s, 1H) 7.46 (m, 4H) 7.55 (m, 1H) 7.88 (d, J=0.78 Hz, 1H) 11.03 (s, 1H).

### EXAMPLE 155

### 8-Chloro-4-(2-chlorophenyl)-7-difluoromethyl-6-methyl-6H-2,6-diaza-as-indacene-1,3-dione

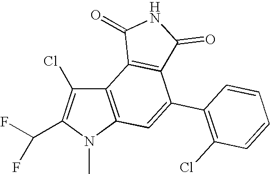

To a solution of 4-(2-chlorophenyl)-7-difluoromethyl-6-methyl-6H-2,6-diaza-as-indacene-1,3-dione (0.1 g) prepared as described in Example 154 in dimethylformamide (4 mL), under an inert atmosphere of dry argon, was added N-chlorosuccinimide (55 mg). The reaction mixture was stirred at room temperature overnight. Ethyl acetate (50 mL) was added and the organic phase was washed with water (3×30 mL), brine (20 mL), dried (MgSO₄) and concentrated to dryness. Crystallization of the sample by vapor diffusion using tetrahydrofuran and hexane yielded the title compound (0.047 g); ¹H NMR (400 MHz, DMSO-D6) δ ppm 3.98 (s, 3H), 7.44 (m, 4H), 7.55 (m, 1H), 8.00 (s, 1H), 11.15 (s, 1H). Mass Spectrum APCI− 396,395,395,393.

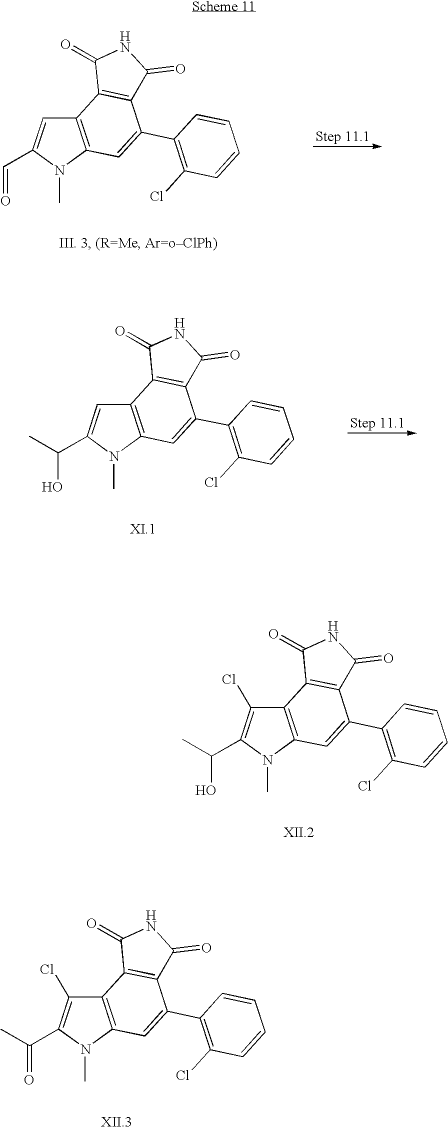

### Procedures for Scheme 11

### EXAMPLE 156

### 4-(2-Chlorophenyl)-7-(1-hydroxyethyl)-6-methyl-6H-2,6-diaza-as-indacene-1,3-dione

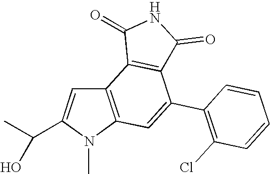

To a solution of 4-(2-chlorophenyl)-6-methyl-1,3-dioxo-1,2,3,6-tetrahydro-2,6-diaza-as-indacene-7-carbaldehyde (0.5 g), prepared as described in Example 72 in tetrahydrofuran (30 mL), at −78° C. under an inert atmosphere of argon was added methylmagnesium bromide [3.0M solution in Et₂O, 0.51 mL]. The reaction mixture was stirred cold for 25 minutes, the cold bath was removed and the reaction mixture was warmed to room temperature. The reaction mixture was stirred for 3 hrs and then methylmagnesium bromide [3.0M solution in Et₂O, 0.51 mL] was added and 30 minutes later a final portion of methylmagnesium bromide [3.0M solution in Et₂O, 0.3 mL] was added. The reaction mixture was stirred for 30 minutes and then saturated ammonium chloride (25 mL) was added. The crude product was extracted with ethyl acetate (3×50 mL), dried (Mg SO₄) and concentrated to dryness. Purification by column chromatography using 5% to 75% ethyl acetate in dichloromethane as the eluent gave the title compound (0.2 g) ¹H NMR (400 MHz, DMSO-D6) ¹H NMR (400 MHz, DMSO-D6) δ ppm 1.56 (d, J=6.45 Hz, 3H), 3.86 (s, 3H), 5.02 (t, J=6.25 Hz, 1H), 5.49 (dd, J=5.96, 3.03 Hz, 1H), 6.77 (s, 1H), 7.43 (m, 3H), 7.53 (m, 1H), 7.67 (d, J=0.78 Hz, 1H), 10.83 (s, 1H).; APCI+ 355,357.

### EXAMPLE 157

### 8-Chloro-4-(2-chlorophenyl)-7-(1-hydroxyethyl)-6-methyl-6H-2,6-diaza-as-indacene-1,3-dione

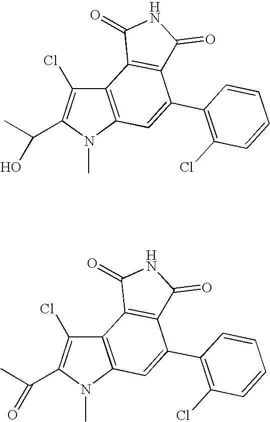

To a solution of 4-(2-chlorophenyl)-7-(1-hydroxyethyl)-6-methyl-6H-2,6-diaza-as-indacene-1,3-dione (50 mg), prepared as described in Example 156, in dimethylformamide (1 mL), under an inert atmosphere of argon was added N-chlorosuccinimide (22 mg). The reaction mixture was stirred at room temperature for 6 h, then poured into ethyl acetate (30 mL) which was washed with water (2×25 mL) and brine (25 mL). The ethyl acetate solution was then dried (MgSO₄) and concentrated to dryness. Purification by column chromatography using a gradient of 5% to 75% ethyl acetate in hexanes as the eluent gave the title compound (33 mg); ¹H NMR (400 MHz, DMSO-D6) 8 ppm) 2.78 (s, 3H) 3.94 (s, 3H) 7.46 (m, 3H) 7.55 (m, 1H), 8.0 (s, 1H) Mass spectrum APCI− 390, 389, 388, 387; and 7-acetyl-8-chloro-4-(2-chlorophenyl)-6-methyl-6H-2,6-diaza-as-indacene-1,3-dione (8 mg). ¹H NMR (400 MHz, DMSO-D6) δ ppm 1.52 (d, J=6.84 Hz, 3H) 4.02 (s, 3H) 5.34 (dd, J=6.94, 3.61 Hz, 1H) 5.84 (dd, J=6.15, 3.61 Hz, 1H) 7.44 (m, 3H) 7.53 (m, 1H) 7.78 (s, 1H) 10.95 (s, 1H); Mass spectrum APCI− 388, 387,386,385.

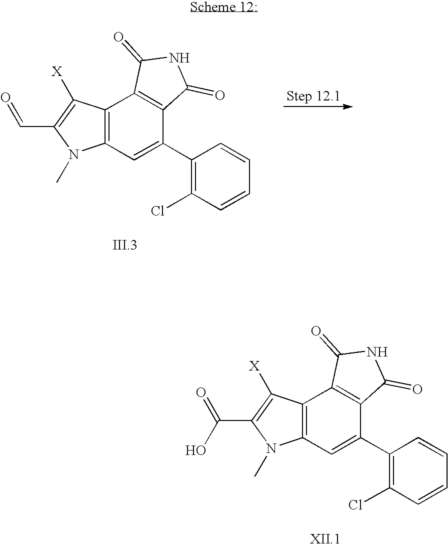

### Procedures for Scheme 12

### EXAMPLE 158

### 8-Bromo-4-(2-chlorophenyl)-6-methyl-1,3-dioxo-1,2,3,6-tetrahydro-2,6-diaza-as-indacene-7-carboxylic acid

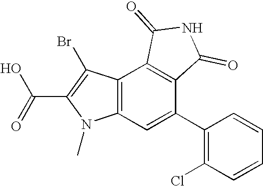

The title compound was prepared from 8-bromo-4-(2-chlorophenyl)-6-methyl-1,3-dioxo-1,2,3,6-tetrahydro-2,6-diaza-as-indacene-7-carbaldehyde, prepared as described in Example 73 (3.25 g, 7.78 mmol), using the procedure described in Experiment 83 to give 3.30 g of product as a yellow solid; ¹H NMR (400 MHz, DMSO-D6) □ ppm 4.02 (s, 3H) 7.45 (m, 3H) 7.54 (m, 1H) 7.95 (s, 1H) 11.06 (d, J=16.84 Hz, 1H) 14.21 (s, 1H); Elemental Analysis for C₁₈H₁₀BrClN₂O₄.0.30H₂O, % C (calc/found) 49.24/49.13, % H 2.43/2.19, % N 6.38/6.31.

### EXAMPLE 159

### 8-Chloro-4-(2-chlorophenyl)-6-methyl-1,3-dioxo-1,2,3,6-tetrahydro-2,6-diaza-as-indacene-7-carboxylic acid

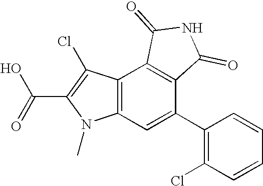

The title compound was prepared from 8-chloro-4-(2-chlorophenyl)-6-methyl-1,3-dioxo-1,2,3,6-tetrahydro-2,6-diaza-as-indacene-7-carbaldehyde, prepared as described in Example 741.02 (0.105 g, 0.28 mmol), using the procedure described in Experiment 83 to give 0.090 g of product as a yellow solid; ¹H NMR (400 MHz, DMSO-D6) δ ppm 4.01 (s, 3H) 7.43 (m, 3H) 7.53 (m, 1H) 7.93 (s, 1H) 11.08 (s, 1H); Elemental Analysis for C₁₈H₁₀Cl₂N₂O₄.0.18H₂O, % C (calc/found) 55.09/54.70, % H 2.66/2.30, % N 7.14/6.77.

### EXAMPLE 160

### 4-(2-Chlorophenyl)-6,8-dimethyl-1,3-dioxo-1,2,3,6-tetrahydro-2,6-diaza-as-indacene-7-carboxylic acid

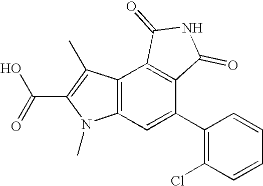

The title compound was prepared from 4-(2-chlorophenyl)-6,8-dimethyl-1,3-dioxo-1,2,3,6-tetrahydro-2,6-diaza-as-indacene-7-carbaldehyde, prepared as described in Example 143 (0.104 g, 0.29 mmol), using the procedure described in Example 83 to give 0.018 g of product as a yellow solid; ¹H NMR (400 MHz, DMSO-D6) δ ppm 2.89 (s, 3H) 4.00 (s, 3H) 7.44 (m, 4H) 7.54 (m, 2H) 7.80 (s, 1H) 11.02 (s, 1H); LCMS (Phenomenex Develosil Combi RP3 50×4.6 mm column, gradient method 570-2% acetonitrile/water eluent (0.1% formic acid) in 2.0 min with 2 min hold, MSD in ESI negative mode) 94% at 3.00 min (UV_{254 nm}); API-ES 323.0 (20), 367.0 (100), 369.0 (50).

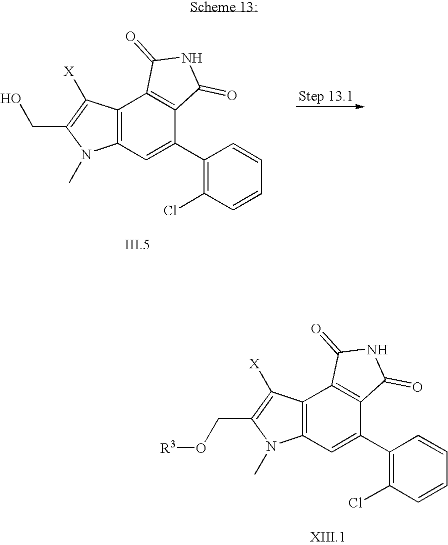

### Procedures for Scheme 13

### EXAMPLE 161

### 4-(2-Chlorophenyl)-7-(2-hydroxyethoxymethyl)-6-methyl-6H-2,6-diaza-as-indacene-1,3-dione

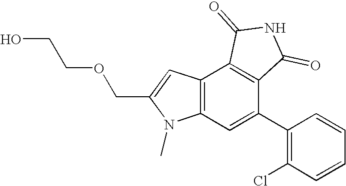

To a solution of 4-(2-chlorophenyl)-7-hydroxymethyl-6-methyl-6H-2,6-diaza-as-indacene-1,3-dione, prepared as described in Example 75 (0.100 g, 0.29 mmol) in ethylene glycol (2.50 mL) was added boron trifluoride etherate (0.416 g, 2.93 mmol). The reaction mixture stirred overnight at 50° C. The reaction mixture was poured into water (25 mL) and the aqueous phase extracted twice with ethyl acetate (25 mL each). The organic phases were combined and washed twice with water (25 mL each), brine (25 mL) and dried over MgSO₄. The organic mixture was concentrated and the crude product purified by flash chromatography (10 g ISCO silica cartridge, ethyl acetate/hexane gradient. Approximately 5 mL fractions were collected) to give 0.064 g of the title compound as a yellow solid; ¹H NMR (400 MHz, DMSO-D6) δ ppm 3.53 (m, 4H) 3.84 (s, 3H) 4.67 (t, J=5.13 Hz, 1H) 4.78 (s, 2H) 6.89 (s, 1H) 7.43 (m, 3H) 7.54 (m, 1H) 7.71 (s, 1H) 10.87 (s, 1H); LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 99% at 1.26 min (UV_{254 nm}); API-ES 323.0 (100), 325.0 (30), 340.9 (10), 385.0 (20).

### EXAMPLE 162

### 4-(2-Chlorophenyl)-7-(2-methoxyethoxymethyl)-6-methyl-6H-2,6-diaza-as-indacene-1,3-dione

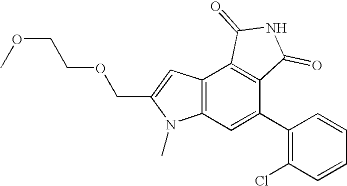

The title compound was prepared from 4-(2-chlorophenyl)-7-hydroxymethyl-6-methyl-6H-2,6-diaza-as-indacene-1,3-dione, prepared as described in Example 75 (0.100 g, 0.29 mmol) and 2-methoxyethanol (2.50 mL), using the procedure described in Example 161 to give 0.003 g of product as a yellow solid; ¹H NMR (400 MHz, DMSO-D6) δ ppm 3.2 (s, 5H) 3.5 (dd, J=5.5, 3.5 Hz, 3H) 3.6 (dd, J=5.6, 3.7 Hz, 2H) 3.8 (s, 3H) 4.8 (s, 2H) 6.9 (s, 1H) 7.4 (m, 3H) 7.5 (d, J=7.6 Hz, 1H) 7.7 (s, 1H) 10.9 (s, 1H); LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 100% at 2.19 min (UV_{254 nm}); API-ES 323.0 (100), 325.0 (30), 399.0 (20).

### EXAMPLE 163

### 8-Chloro-4-(2-chlorophenyl)-7-(2-hydroxyethoxymethyl)-6-methyl-6H-2,6-diaza-as-indacene-1,3-dione

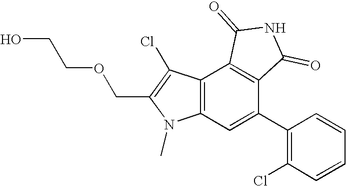

To a solution of 8-chloro-4-(2-chlorophenyl)-7-hydroxymethyl-6-methyl-6H-2,6-diaza-as-indacene-1,3-dione, prepared as described in Example 77 (0.100 g, 0.27 mmol) in ethylene glycol (1.00 mL) was added concentrated sulfuric acid (one drop). The reaction mixture stirred overnight at 50° C. The reaction mixture was poured into water (25 mL) and the aqueous phase extracted twice with ethyl acetate (25 mL each). The organic phases were combined and washed twice with water (25 mL each), brine (25 mL) and dried over MgSO₄. The organic mixture was concentrated and the crude product purified by flash chromatography (10 g ISCO silica cartridge, ethyl acetate/hexane gradient. Approximately 5 mL fractions were collected) to give 0.050 g of the title compound as a yellow solid; ¹H NMR (400 MHz, DMSO-D6) δ ppm 3.5 (m, 4H) 3.9 (s, 3H) 4.7 (m, 1H) 4.8 (s, 2H) 7.4 (s, 3H) 7.5 (s, 1H) 7.9 (s, 1H) 11.0 (s, 1H); Elemental Analysis for C₂₀H₁₆Cl₂N₂O₄% C (calc/found) 57.30/57.23, % H 3.85/3.45, % N 6.68/6.56.

### EXAMPLE 164

### 8-Bromo-4-(2-chlorophenyl)-7-(2-hydroxyethoxymethyl)-6-methyl-6H-2,6-diaza-as-indacene-1,3-dione

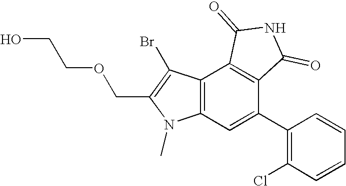

The title compound was prepared from 8-bromo-4-(2-chlorophenyl)-7-hydroxymethyl-6-methyl-6H-2,6-diaza-as-indacene-1,3-dione, prepared as described in Example 76 (0.250 g, 0.60 mmol), boron trifluoride etherate (1.154 g, 8.131 mmol) and ethylene glycol (5.00 mL), using the procedure described in DS XII 1_{—}3.01 to give 0.051 g of product as a yellow solid; ¹H NMR (400 MHz, DMSO-D6) δ ppm 3.51 (m, 4H) 3.90 (s, 3H) 4.66 (d, J=4.88 Hz, 1H) 4.83 (s, 2H) 7.44 (m, 3H) 7.54 (m, 1H) 7.85 (s, 1H) 10.98 (s, 1H); LCMS (Phenomenex Develosil Combi RP3 50×4.6 mm column, gradient method 50-2% acetonitrile/water eluent (0.1% formic acid) in 2.0 min with 2 min hold, MSD in ESI positive mode) 100% at 1.61 min (UV_{214 nm}); API-ES 400.9 (80), 402.9 (100), 403.9 (35), 464.8 (20).

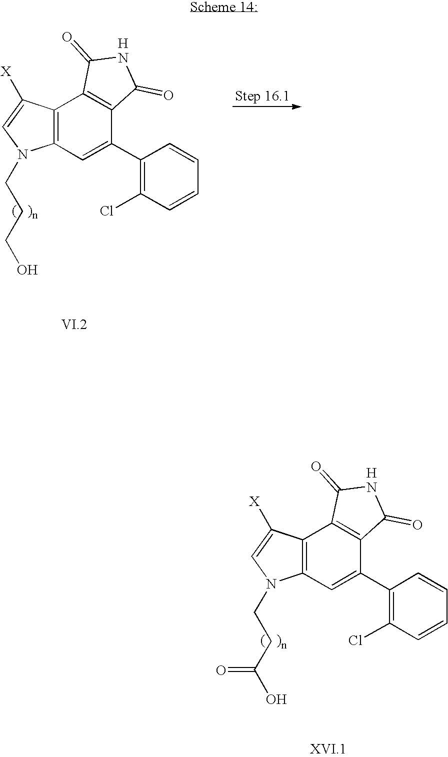

### Procedures for Scheme 14

### EXAMPLE 165

### 3-[8-Bromo-4-(2-chlorophenyl)-1,3-dioxo-2,3-dihydro-1H-2,6-diaza-as-indacen-6-yl]propionic acid

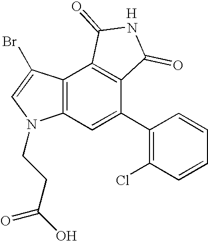

Jones reagent (ca. 1.5 mL, excess) was added dropwise at room temperature to a stirred solution of 8-bromo-4-(2-chlorophenyl)-6-(3-hydroxypropyl)-6H-2,6-diaza-as-indacene-1,3-dione, prepared as described in Example 45 (0.20 g, 0.46 mmol) in acetone (25 mL). After 15 min the mixture was diluted with water, extracted with ethyl acetate and the organic solution worked up to give a solid which was chromatographed on silica. Elution with ethyl acetate gave foreruns, while methanol/ethyl acetate (1:19) eluted the title compound (40.1 mg, 19%), which crystallised from ethyl acetate/petroleum ether as a yellow powder, mp 182-186° C. ¹H NMR (400 MHz, DMSO-D6) δ ppm 2.79 (t, J=6.7 Hz, 2H), 4.51 (t, J=6.7 Hz, 2H), 7.40-7.48 (m, 3H), 7.52-7.56 (m, 1H), 7.90 (s, 1H), 7.98 (s, 1H), 10.92 (s, 1H), 12.37 (br, 1H). API-CI Found: 447, 449, 450 (M+1). Elemental Analysis for C₁₉H₁₂BrClN₂O₄.¼H₂O, % C (calc/found) 50.47/50.30, % H 2.79/3.07, % N 6.19/6.27.

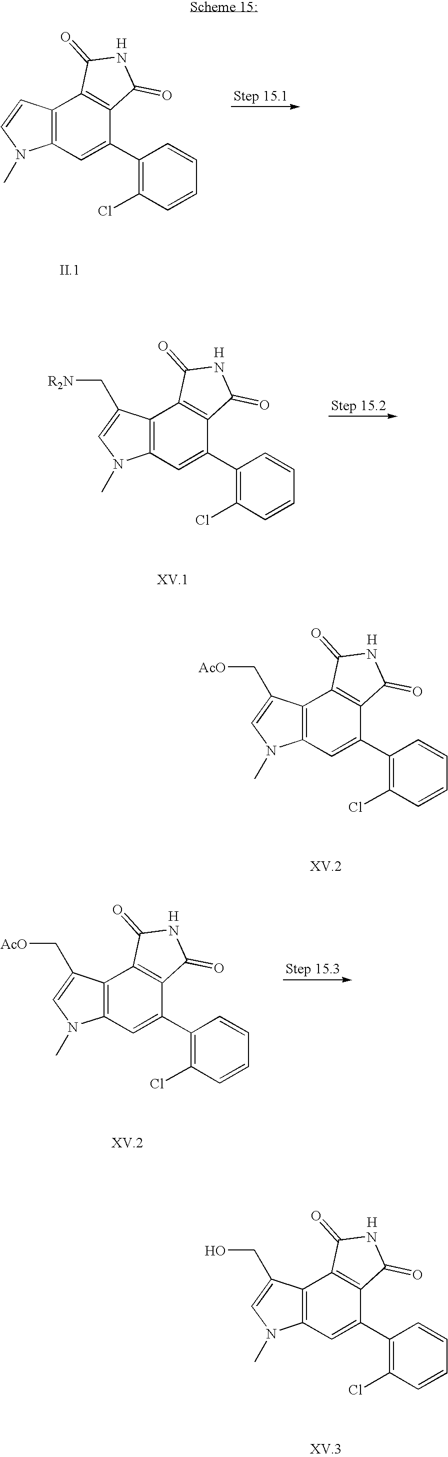

### Procedures for Scheme 15

### EXAMPLE 166

### 4-(2-Chlorophenyl)-6-methyl-8-pyrrolidin-1-ylmethyl-6H-2,6-diaza-as-indacene-1,3-dione

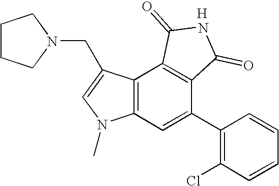

A solution of 4-(2-chlorophenyl)-6-methyl-6H-2,6-diaza-as-indacene-1,3-dione, prepared as described in Example 29 (0.10 g, 0.32 mmol) in acetic acid (8 mL) and tetrahydrofuran (2 mL) was warmed to 50° C. Formaldehyde (54.4 μL of a 40% w/v aqueous solution, 0.48 mmol) and pyrrolidine (40 μL, 0.48 mmol) were added and the solution was warmed for 2 h. The solution was concentrated to dryness in vacuo, then partitioned between ethyl acetate and water. The organic solution was washed well with water, saturated aqueous NaHCO₃ solution, then dried Na₂SO₄ and concentrated to dryness. The residue was adsorbed onto silica and chromatographed. Elution with ethyl acetate gave foreruns, while conc. aqueous ammonia/methanol/ethyl acetate (1:5:44) eluted the title compound (83.4 mg, 66%), which crystallised from tetrahydrofuran/petroleum ether as a yellow powder, mp 160-165° C. (dec.). ¹H NMR (400 MHz, DMSO-D6) δ ppm 1.67-1.81 (br s, 4H), 2.61-2.80 (br, 4H), 3.87 (s, 3H), 4.18-4.40 (br, 2H), 7.41-7.47 (m, 3H), 7.52-7.56 (m, 1H), 7.67 (br s, 1H), 7.72 (br, 1H), 10.88 (br s, 1H). API-CI Found: 394, 396 (M+1).

### EXAMPLE 167

### 4-(2-Chlorophenyl)-8-dimethylaminomethyl-6-methyl-6H-2,6-diaza-as-indacene-1,3-dione

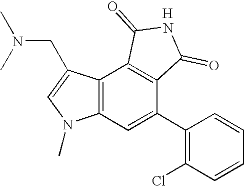

Reaction of 4-(2-chlorophenyl)-6-methyl-6H-2,6-diaza-as-indacene-1,3-dione, prepared as described in Example 29 with aqueous dimethylamine and formaldehyde in acetic acid and tetrahydrofuran as described in Example 166, except that the reaction time was 8 h at 50° C. gave the title compound (58%) which crystallised from tetrahydrofuran/petroleum ether as a yellow powder, mp 155-159° C. ¹H NMR (400 MHz, DMSO-D6) δ ppm 2.20 (s, 6H), 3.86 (s, 3H), 3.89 (d, J=13.4 Hz, 1H), 3.95 (d, J=13.4 Hz, 1H), 7.41-7.47 (m, 3H), 7.52-7.56 (m, 1H), 7.63 (s, 1H), 7.64 (s, 1H), 10.81 (br s, 1H). API-CI Found: 368, 370 (M+1).

### EXAMPLE 168

### Acetic acid 4-(2-chlorophenyl)-6-methyl-1,3-dioxo-1,2,3,6-tetrahydro-2,6-diaza-as-indacen-8-ylmethyl ester (XV.2.1) and 4-(2-chlorophenyl)-8-hydroxymethyl-6-methyl-6H-2,6-diaza-as-indacene-1,3-dione

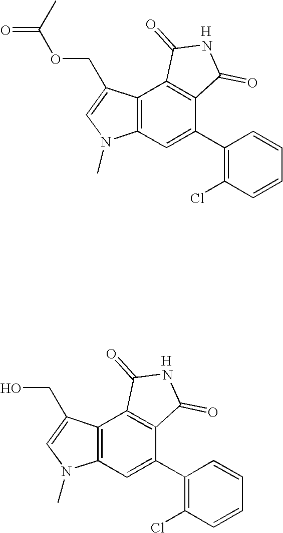

A solution of 4-(2-chlorophenyl)-6-methyl-8-pyrrolidin-1-ylmethyl-6H-2,6-diaza-as-indacene-1,3-dione, prepared as described in Example 166 (0.30 g, 0.76 mmol) in acetic anhydride (40 mL) was refluxed for 2 h, then concentrated to dryness in vacuo. The residue was partitioned between ethyl acetate and saturated aqueous NaHCO₃ solution and the organic portion was worked up to give crude acetic acid 4-(2-chlorophenyl)-6-methyl-1,3-dioxo-1,2,3,6-tetrahydro-2,6-diaza-as-indacen-8-ylmethyl ester as a yellow solid, which was used directly. API-CI Found: 383, 384 (M+1). 2N K₂CO₃ solution (3 mL) was added to a solution of the crude acetate (0.20 g, 0.52 mmol) in methanol/tetrahydrofuran (2:1) (20 mL) and the mixture was stirred at room temperature for 3 h. After dilution with water the mixture was extracted into ethyl acetate and worked up to give an oil which was chromatographed on silica. Elution with ethyl acetate gave 4-(2-chlorophenyl)-8-hydroxymethyl-6-methyl-6H-2,6-diaza-as-indacene-1,3-dione (50.7 mg, 28%) which crystallised from tetrahydrofuran/petroleum ether as a yellow solid, mp 270-274° C. ¹H NMR (400 MHz, DMSO-D6) δ ppm 3.86 (s, 3H), 4.86-4.93 (m, 3H), 7.41-7.47 (m, 3H), 7.52-7.56 (m, 1H), 7.65 (br s, 2H), 10.91 (br, 1H). ¹H NMR (400 MHz, DMSO-D6 plus D₂O) δ ppm 3.57 (s, 3H), 4.87 (s, 2H), 7.38-7.44 (m, 3H), 7.47-7.52 (m, 1H), 7.56 (s, 1H), 7.60 (s, 1H). API-CI 339, 341 (M-1). Elemental Analysis for C₁₈H₁₃ClN₂O₃, % C (calc/found) 63.44/63.91, % H 3.84/3.90, % N 8.22/8.44.

1. A compound of Formula I: 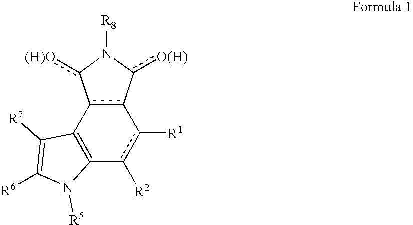
wherein:
each dashed line represents an optional bond;
R¹ is selected from hydrogen, halogen, alkyl; or
a) a phenyl or heteroaryl ring optionally substituted with up to five substituents selected from halogen, alkyl, nitro, CN, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₘR⁴, NHCONR³R⁴, NR³CONHR⁴; or
b) a cycloalkyl or cycloalkenyl ring optionally substituted with up to five substituents selected from, halogen, alkyl, nitro, CN, C(O)R³, OR³, S(O)ₘR³, NR³R⁴, OC(O)R³, NR³(CO)OR⁴, CH₂NR³R⁴, CH₂OR³ COOR³, CONR³R⁴, NR³COR⁴, SO₂NR³R⁴, CONHSO₂R³, NR₃S(O)ₘR⁴, NHCONR³R⁴, NR³CONHR⁴; or
c) a heterocyclic ring optionally substituted with up to five substituents selected from, halogen, alkyl, nitro, CN, C(O)R³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR₃S(O)ₘR⁴ NHCONR³R⁴, or NR³CONHR⁴;
R² is selected from hydrogen, halogen, alkyl; or a cycloalkyl or cycloalkenyl ring optionally substituted with up to five substituents selected from halogen, alkyl, hydroxyl, nitro, cyano, C(O)R³, OR³, S(O)ₘR³, NR³R⁴, OC(O)R³, NR³(CO)OR⁴, CH₂NR³R⁴, CH₂OR³ COOR³, CONR³R⁴, NR³COR⁴, SO₂NR³R⁴, CONHSO₂R³, NR₃S(O)ₚR⁴, NHCONR³R⁴, or NR³CONHR⁴; or
R³ and R⁴ are independently selected from hydrogen, alkyl, or a substituted or unsubstituted carbocyclic group selected from cycloalkyl, cycloalkenyl, heterocyclyl, aryl, and heteroaryl, wherein the said alkyl, or a substituted or unsubstituted carbocyclic group may be substituted with up to 4 groups selected from halogen, hydroxyl, nitro, cyano, alkyl, alkoxy, carboxy, C(O)OH, CONH₂, NHC(O)CH₃, N(CH₃)₂, NHCH₃ thiomethyl, thioethyl, SOCH₃, SO₂CH₃, NHC(O)NR³R⁴, NR³C(O)NHR⁴; or
R³ and R⁴ together with the carbon atom or heteroatom to which they are attached may form a cycloalkyl or heterocyclyl group optionally substituted with up to 4 groups independently selected from halo, hydroxyl, nitro, CN, alkyl, alkyloxy, formyl, carboxy, acetyl, CH₂NH₂, CH₂OH, COOH, CONH₂, NHCOCH₃, N(CH₃)₂, thiomethyl, thioethyl, SOCH₃, SO₂CH₃, alkoxycarbonyl, alkylcarbonyl, alknylamino, aminoalkyl, aminoalkylcarbonyl, amino, mono- or dialkylamino; or
R³ and R⁴ together with the nitrogen to which they are attached may form a heterocyclic ring containing 3-8 members, up to four of which members are optionally carbonyl groups or heteroatoms independently selected from oxygen, sulfur, S(O), S(O)₂, and nitrogen, wherein the carbocyclic group is unsubstituted or substituted with up to four groups independently selected from halogen, hydroxy, hydroxyalkyl, alkyl, alkoxy, alkoxycarbonyl, alkylcarbonyl, alknylamino, aminoalkyl, aminoalkylcarbonyl, amino, mono- or dialkylamino,
R⁵ is selected from hydrogen, alkyl or ((CR⁹R¹⁰)ₙT)ₐ(CR¹¹R¹²)_{b})-Z;
T may be absent, or, when present, is in each instance independently selected from O, CONR³, CONHSO₂, S, S(O)ₘ, NR³, NR³—O, O—S(O)ₘ, S(O)ₘ—O, NR³—S(O)₂, or S(O)₂—NR³;
m=0, 1, 2;
n, a and b are in each instance independently 0-6;
p=1 or 2;
Z is selected from hydrogen, halogen, alkyl, cycloalkyl, cycloalkenyl, heterocyclyl, phenyl, heteroaryl, halogen, cyano, nitro, hydroxy, COR³, CONHSO₂R³, OR³, S(O)ₘR³OSO₂R³, NR³R⁴, CO₂R³, CONR³R⁴, NR³COR⁴, SO₂NR³R⁴, OPO(OR³)(OR⁴), CH═CR³R⁴, CCR³, wherein the alkyl, cycloalkyl, cycloalkenyl, heterocyclyl, phenyl or heteroaryl group may be optionally substituted with up to four groups independently selected from halogen, alkyl, hydroxyl, nitro, cyano, OR³, S(O)ₘR³, NR³R⁴, OC(O)R³, NR³(CO)OR⁴, C(O)R³, COOR³, CONR³R⁴, NR³COR⁴, SO₂NR³R⁴, CONHSO₂R³, NR₃S(O)ₚR⁴, CH₂NR³R⁴, CH₂OR³ NHCONR³R⁴, NR³CONHR⁴;
R⁶ is selected from hydrogen, halogen, CN, alkyl, CH₂OR³, CR⁹═CR¹¹R¹², C≡CR⁹, or ((CR⁹R¹⁰)ₙT)ₐ(CR¹, R¹²)_{b})-Z; where T, Z, n, a and b are defined as above; or —NC(O)—R³;
a) a phenyl or heteroaryl ring optionally substituted with up to five substituents selected from halogen, alkyl, nitro, CN, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₚR⁴ NHCONR³R⁴, NR³CONHR⁴; or
b) a cycloalkyl or cycloalkenyl ring optionally substituted with up to 5 substituents selected from, halo, alkyl, nitro, CN, C(O)R³, OR³, S(O)ₘR³, NR³R⁴, OC(O)R³, NR³(CO)OR⁴, CH₂ NR³R⁴, CH₂OR³ COOR³, CONR³R⁴, NR³COR⁴, SO₂NR³R⁴, CONHSO₂R³, NR₃S(O)ₚR⁴, NHCONR³R⁴, or NR³CONHR³R⁴; or
c) a heterocyclic ring optionally substituted with up to five substituents selected from, halo, alkyl, nitro, CN, C(O)R³, OR³, S(O)ₘR³, NR³R⁴, OC(O)R³ NR³(CO)OR⁴, CH₂NR³R⁴, CH₂OR³, COOR³, CONR³R⁴, NR³COR⁴, SO₂NR³R⁴, CONHSO₂R³, NR₃S(O)ₚR⁴ NHCONR³R⁴, or NR³CONHR⁴;
R⁷ is selected from hydrogen, halogen, CN, alkyl, CR⁹═CR¹¹R¹², CCR⁹, or ((CR⁹R¹⁰)ₙT)ₐ(CR¹¹R¹²)_{b})-Z; where T, Z, n, a and b are defined as above;
R⁸ is selected from hydrogen or C₁-C₃ alkyl;
R⁹, R¹⁰, R¹¹ and R¹² are in each instance independently selected from hydrogen, hydroxyl, alkyl, cycloalkyl, cycloalkenyl, phenyl, heteroaryl, heterocyclyl, halogen, cyano, nitro, CH₂NR³R⁴, CH₂OR³, COR³, OR³, S(O)ₘR³, NR³R⁴, COOR³, CONR³R⁴, SO₂NR³R⁴NHCONR³R⁴, NR³CONHR⁴;
wherein the alkyl, cycloalkyl, cycloalkenyl, heterocyclyl, phenyl or heteroaryl group may be optionally substituted with up to 4 groups independently selected from halogen, alkyl, nitro, cyano, OR³, S(O)ₘR³, NR³R⁴, OC(O)R³, NR³(CO)OR⁴, C(O)R³, COOR³, CONR³R⁴, NR³COR⁴, SO₂NR³R⁴, CONHSO₂R³, NR₃S(O)ₘR⁴. NHCONR³R⁴, NR³CONHR⁴;
R⁵ and R⁶ or R¹¹ and R¹² together with the carbon atom to which they are attached may form a carbonyl group; or a cycloalkyl or heterocyclyl group, optionally substituted with up to four groups independently selected from halogen, hydroxyl, nitro, cyano, alkyl, halogen, alkyl, nitro, OR³, S(O)ₘR³, NR³R⁴, OC(O)R³, NR³(CO)OR⁴, C(O)R³, COOR³, CONR³R⁴, NR³COR⁴, SO₂NR³R⁴, CONHSO₂R³, NR₃S(O)ₘR⁴, NHCONR³R⁴, NR³CONHR⁴; or a pharmaceutically acceptable salt form thereof. A compound of claim 1 wherein:
R¹ is phenyl optionally substituted by up to three substituents selected from halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₘR⁴, NHCONR³R⁴, NR³CONHR³R⁴;
R³ and R⁴ are as defined in claim 1;
R⁵ is a C₁-C₆ alkyl group optionally substituted by from 1 to 3 groups selected from OH, halogen, —NR³R⁴, —O—(C₁-C₃ alkyl), —O—(C₁-C₃ alkyl)-O—(C₁-C₃ alkyl), with the C₁-C₃ alkyl portions of the last two groups being further optionally substituted by OH, halogen or —NR³R⁴;
R⁶ is H, halogen, C₁-C₆ alkyl, C₁-C₆ alkenyl, —C(O)OH, —C(O)—NR³R⁴, —C(O)—(C₁-C₆ alkyl), —(C₁-C₆ alkyl)-NH—(C₁-C₆ alkyl), —(C₁-C₆ alkyl)-O—(C₁-C₆ alkyl), —(C₁-C₆ alkyl)-O—(C₁-C₆ alkyl)-O—(C₁-C₆ alkyl),
with the alkyl portions of each R⁶ group containing an alkyl or alkenyl portion being further optionally substituted by from 1 to 3 groups selected from CN, OH, halo, —C(O)OH, C(O)O(C₁-C₆ alkyl), which may also include a single —NR³R⁴ or a phenyl, heterocyclyl or heteroaryl group optionally substituted by from 1 to 3 groups selected from halo, alkyl, nitro, CN, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₚR⁴ NHCONR³R⁴, or NR³CONHR⁴;
R⁷ is H, halogen, C₁-C₆ alkyl, C₁-C₆ alkenyl, C₁-C₆ alkynyl, the alkyl, alkenyl and alkynyl groups being optionally substituted by from 1 to 3 groups selected from oxo, OH, CN, or —NR³R⁴; and
R⁸ is selected from hydrogen or C₁-C₃ alkyl;
or a pharmaceutically acceptable salt or ester form thereof.

2. A compound of claim 1 wherein:
R¹ is phenyl optionally substituted by up to three substituents selected from halogen, alkyl, hydroxyl, nitro, cyano, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₘR⁴, NHCONR³R⁴, NR³CONHR⁴;
R³ and R⁴ are as defined in claim 1;
R⁵ is a C₁-C₆ alkyl group optionally substituted by from 1 to 3 groups selected from OH, halogen, 4-O—(C₁-C₃ alkyl), —O—(C₁-C₃ alkyl)-O—(C₁-C₃ alkyl), with the C₁-C₃ alkyl portions of the last two groups being further optionally substituted by OH, halogen or —NR³R⁴;
R⁶ is H, halogen, C₁-C₆ alkyl, C₁-C₆ alkenyl, —C(O)OH, —C(O)—NR³R⁴, —C(O)—(C₁-C₆ alkyl), —(C₁-C₆ alkyl)-NH—(C₁-C₆ alkyl), —(C₁-C₆ alkyl)-O—(C₁-C₆ alkyl), —(C₁-C₆ alkyl)-O—(C₁-C₆ alkyl)-O—(C₁-C₆ alkyl),
with the alkyl portions of each R⁶ group containing an alkyl or alkenyl portion being further optionally substituted by from 1 to 3 groups selected from CN, OH, halo, —C(O)OH, C(O)O(C₁-C₆ alkyl), which may also include a single —NR³R⁴ or a phenyl, heterocyclyl or heteroaryl group optionally substituted by from 1 to 3 groups selected from halo, alkyl, nitro, CN, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³ NR³S(O)ₚR⁴ NHCONR³R⁴, or NR³CONHR⁴;
R⁷ is H, halogen, C₁-C₆ alkyl, C₁-C₆ alkenyl, C₁-C₆ alkynyl, the alkyl, alkenyl and alkynyl groups being optionally substituted by from 1 to 3 groups selected from oxo, OH, CN, or —NR³R⁴; and
R⁸ is selected from hydrogen or C₁-C₃ alkyl;
or a pharmaceutically acceptable salt or ester form thereof.

3. A compound of claim 1 wherein R¹ is a phenyl group ortho-substituted by halogen, —O—C₁-C₃ alkyl, OH, NO₂, NH₂, NH(C₁-C₃ alkyl), N(C₁-C₃ alkyl)₂, or CN, or a pharmaceutically acceptable salt form thereof.

4. A compound of claim 3 wherein R⁸ is hydrogen.

5. A compound of Formula II: 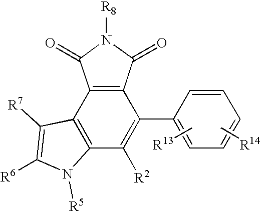
wherein:
R² is H or C₁-C₃ alkyl;
R⁵ is H or C₁-C₆ alkyl optionally substituted by OH, halogen or NR³R⁴;
R³ and R⁴ are as defined in claim 1;
R¹³ and R¹⁴ are independently selected from H, halo, alkyl, nitro, CN, COR³, OR³, S(O)ₘR³, NR³R⁴, OC(O)R³, NR³(CO)OR⁴, CH₂NR³R⁴, CH₂OR³, COOR³, CONR³R⁴, NR³COR⁴, SO₂NR³R⁴, CONHSO₂R³, NR³S(O)ₘR⁴, NHCONR³R⁴, or NR³CONHR⁴;
R⁶ is H, halo, C₁-C₆ alkyl, C₁-C₆ alkenyl, —C(O)OH, —C(O)—NR³R⁴, —C(O)—(C₁-C₆ alkyl), —(C₁-C₆ alkyl)-NH—(C₁-C₆ alkyl), —(C₁-C₆ alkyl)-O—(C₁-C₆ alkyl), —(C₁-C₆ alkyl)-O—(C₁-C₆ alkyl)-O—(C₁-C₆ alkyl); the alkyl portions of each R⁶ group containing an alkyl or alkenyl portion being further optionally substituted by from 1 to 3 groups selected from CN, OH, halo, —C(O)OH, C(O)O(C₁-C₆ alkyl), which may also include a single —NR³R⁴ or a phenyl, heterocyclyl or heteroaryl group optionally substituted by from 1 to 3 groups selected from halo, alkyl, nitro, CN, COR³, OR³, S(O)ₘR, NR³R⁴, OC(O)R³, NR³ (CO)OR³, CH₂NR³R⁴, CH₂OR³, COOR³, CONR³R⁴, NR³COR⁴, SO₂NR³R⁴, CONHSO₂R³, NR³S(O)ₚR⁴ NHCONR³R⁴, or NR³CONHR⁴;
R⁷ is H, halogen, C₁-C₆ alkyl, C₁-C₆ alkenyl, C₁-C₆ alkynyl, the alkyl, alkenyl and alkynyl groups being optionally substituted by from 1 to 3 groups selected from oxo, OH, CN, or —NR³R⁴; and
R⁸ is H or —CH₃;
or a pharmaceutically acceptable salt form thereof.

6. A compound of claim 5 wherein R¹ is a phenyl group ortho-substituted by halogen, —O—C₁-C₃ alkyl, OH, NO₂, NH₂, NH(C₁-C₃ alkyl), N(C₁-C₃ alkyl)₂, or CN, or a pharmaceutically acceptable salt form thereof.

7. A compound of claim 6 wherein R⁸ is hydrogen.

8. A compound of claim 1 selected from the group of:
4-(2-Chlorophenyl)-6-methyl-4,5,6,8b-tetrahydro-3aH-2,6-diaza-as-indacene-1,3-dione;
4-(2-Methoxyphenyl)-6-methyl-4,5,6,8b-tetrahydro-3aH-2,6-diaza-as-indacene-1,3-dione;
4-(2-Chloro-6-methoxyphenyl)-6-methyl-4,5,6,8b-tetrahydro-3aH-2,6-diaza-as-indacene-1,3-dione;
4-(2-Chlorophenyl)-6-[2-(2-ethoxyethoxy)ethyl]-4,5,6,8b-tetrahydro-3aH-2,6-diaza-as-indacene-1,3-dione;
4-(2-Chlorophenyl)-6-(2-methoxyethyl)-4,5,6,8b-tetrahydro-3aH-2,6-diaza-as-indacene-1,3-dione;
4-(2-Chlorophenyl)-6-(3-methoxypropyl)-4,5,6,8b-tetrahydro-3aH-2,6-diaza-as-indacene-1,3-dione;
4-(2-Chlorophenyl)-6-[3-(2-methoxyethoxy)propyl]-4,5,6,8b-tetrahydro-3aH-2,6-diaza-as-indacene-1,3-dione;
4-(2-Chlorophenyl)-6-(3-methoxypropyl)-1,3-dioxo-1,2,3,3a,4,5,6,8b-octahydro-2,6-diaza-as-indacene-7-carboxylic acid; 6-[2-(tert-Butyldimethylsilanyloxy)ethyl]-4-(2-methoxyphenyl)-4,5,6,8b-tetrahydro-3aH-2,6-diaza-as-indacene-1,3-dione;
4-(2-Methoxyphenyl)-6-methyl-6H-2,6-diaza-as-indacene-1,3-dione;
8-Bromo-4-(2-methoxyphenyl)-6-methyl-6H-2,6-diaza-as-indacene-1,3-dione;
4-(2-Chlorophenyl)-6-methyl-6H-2,6-diaza-as-indacene-1,3-dione;
8-Chloro-4-(2-methoxyphenyl)-6-methyl-6H-2,6-diaza-as-indacene-1,3-dione;
8-Bromo-6-methyl-4-(2-methylsulfanylphenyl)-6H-2,6-diaza-as-indacene-1,3-dione;
4-(2-Chlorophenyl)-8-iodo-6-methyl-6H-2,6-diaza-as-indacene-1,3-dione;
4-(2-Chloro-6-methoxyphenyl)-6-methyl-6H-2,6-diaza-as-indacene-1,3-dione;
8-Bromo-4-(2-chloro-6-methoxyphenyl)-6-methyl-6H-2,6-diaza-as-indacene-1,3-dione;
4-(2-Chlorophenyl)-6-[2-(2-ethoxyethoxy)ethyl]-6H-2,6-diaza-as-indacene-1,3-dione;
8-Bromo-4-(2-chlorophenyl)-6-[2-(2-ethoxyethoxy)ethyl]-6H-2,6-diaza-as-indacene-1,3-dione;
8-Bromo-4-(2-chlorophenyl)-6-methyl-6H-2,6-diaza-as-indacene-1,3-dione;
or a pharmaceutically acceptable salt, amide or ester form thereof.

9. A compound of claim 1 selected from the group of:
4-(2-Chlorophenyl)-6-(2-methoxyethyl)-6H-2,6-diaza-as-indacene-1,3-dione;
8-Bromo-4-(2-chlorophenyl)-6-(2-methoxyethyl)-6H-2,6-diaza-as-indacene-1,3-dione;
8-Bromo-4-(2-chlorophenyl)-6-(2-hydroxyethyl)-6H-2,6-diaza-as-indacene-1,3-dione;
6-[3-(tert-Butyldimethylsilanyloxy)propyl]-4-(2-chlorophenyl)-6H-2,6-diaza-as-indacene-1,3-dione;
4-(2-Chlorophenyl)-6-(3-hydroxypropyl)-6H-2,6-diaza-as-indacene-1,3-dione;
4-(2-Chlorophenyl)-6-(3-methoxypropyl)-6H-2,6-diaza-as-indacene-1,3-dione;
8-Bromo-4-(2-chlorophenyl)-6-(3-methoxypropyl)-6H-2,6-diaza-as-indacene-1,3-dione;
8-Bromo-6-(3-bromopropyl)-4-(2-chlorophenyl)-6H-2,6-diaza-as-indacene-1,3-dione;
8-Bromo-4-(2-chlorophenyl)-6-(3-hydroxypropyl)-6H-2,6-diaza-as-indacene-1,3-dione;
4-(2-Chlorophenyl)-6-(3-methoxypropyl)-1,3-dioxo-1,2,3,6-tetrahydro-2,6-diaza-as-indacene-7-carboxylic acid;
4-(2-Chlorophenyl)-6-(3-methoxypropyl)-1,3-dioxo-1,2,3,6-tetrahydro-2,6-diaza-as-indacene-7-carboxylic acid;
6-(3-Bromopropyl)-4-(2-chlorophenyl)-1,3-dioxo-1,2,3,6-tetrahydro-2,6-diaza-as-indacene-7-carboxylic acid;
4-(2-Chlorophenyl)-6-(3-hydroxypropyl)-1,3-dioxo-1,2,3,6-tetrahydro-2,6-diaza-as-indacene-7-carboxylic acid;
6-(3-Bromopropyl)-4-(2-chlorophenyl)-1,3-dioxo-1,2,3,6-tetrahydro-2,6-diaza-as-indacene-7-carboxylic acid;
6-(3-Bromopropyl)-4-(2-chlorophenyl)-7-hydroxymethyl-6H-2,6-diaza-as-indacene-1,3-dione;
4-(2-Chlorophenyl)-6-(3-methoxypropyl)-1,3-dioxo-1,2,3,6-tetrahydro-2,6-diaza-as-indacene-7-carboxylic acid;
4-(2-Chlorophenyl)-6-(2-methoxyethyl)-1,3-dioxo-1,2,3,3a,4,5,6,8b-octahydro-2,6-diaza-as-indacene-7-carbaldehyde;
4-(2-Chlorophenyl)-6-(2-methoxyethyl)-1,3-dioxo-1,2,3,6-tetrahydro-2,6-diaza-as-indacene-7-carbaldehyde;
4-(2-Chlorophenyl)-6-[2-(2-ethoxyethoxy)ethyl]-1,3-dioxo-1,2,3,3a,4,5,6,8b-octahydro-2,6-diaza-as-indacene-7-carbaldehyde;
4-(2-Chlorophenyl)-6-[2-(2-ethoxyethoxy)ethyl]-1,3-dioxo-1,2,3,6-tetrahydro-2,6-diaza-as-indacene-7-carbaldehyde;
or a pharmaceutically acceptable salt, amide or ester form thereof.

10. A compound of claim 1 selected from the group of:
8-Bromo-4-(2-chlorophenyl)-6-[2-(2-ethoxyethoxy)ethyl]-1,3-dioxo-1,2,3,6-tetrahydro-2,6-diaza-as-indacene-7-carbaldehyde;
4-(2-Chlorophenyl)-6-(3-methoxypropyl)-1,3-dioxo-1,2,3,3a,4,5,6,8b-octahydro-2,6-diaza-as-indacene-7-carbaldehyde;
4-(2-Chlorophenyl)-6-(3-methoxypropyl)-1,3-dioxo-1,2,3,6-tetrahydro-2,6-diaza-as-indacene-7-carbaldehyde;
4-(2-Chlorophenyl)-7-hydroxymethyl-6-(3-methoxypropyl)-6H-2,6-diaza-as-indacene-1,3-dione;
8-Bromo-4-(2-chlorophenyl)-7-hydroxymethyl-6-(3-methoxypropyl)-6H-2,6-diaza-as-indacene-1,3-dione;
8-Bromo-4-(2-chlorophenyl)-6-(3-methoxypropyl)-1,3-dioxo-1,2,3,6-tetrahydro-2,6-diaza-as-indacene-7-carbaldehyde;
8-Bromo-6-(3-bromopropyl)-4-(2-chlorophenyl)-1,3-dioxo-1,2,3,6-tetrahydro-2,6-diaza-as-indacene-7-carbaldehyde;
8-Bromo-4-(2-chlorophenyl)-6-(3-hydroxypropyl)-1,3-dioxo-1,2,3,6-tetrahydro-2,6-diaza-as-indacene-7-carbaldehyde;
8-Chloro-4-(2-chlorophenyl)-6-(3-methoxypropyl)-1,3-dioxo-1,2,3,6-tetrahydro-2,6-diaza-as-indacene-7-carbaldehyde;
8-Chloro-6-(3-bromopropyl)-4-(2-chlorophenyl)-1,3-dioxo-1,2,3,6-tetrahydro-2,6-diaza-as-indacene-7-carbaldehyde;
8-Chloro-4-(2-chlorophenyl)-6-(3-hydroxypropyl)-1,3-dioxo-1,2,3,6-tetrahydro-2,6-diaza-as-indacene-7-carbaldehyde;
8-Chloro-4-(2-chlorophenyl)-7-hydroxymethyl-6-(3-hydroxypropyl)-6H-2,6-diaza-as-indacene-1,3-dione;
8-Chloro-4-(2-chlorophenyl)-7-hydroxymethyl-6-(3-methoxypropyl)-6H-2,6-diaza-as-indacene-1,3-dione;
4-(2-Chlorophenyl)-6-[3-(2-methoxyethoxy)propyl]-1,3-dioxo-1,2,3,6-tetrahydro-2,6-diaza-as-indacene-7-carbaldehyde;
8-Bromo-4-(2-chlorophenyl)-6-[3-(2-methoxyethoxy)propyl]-1,3-dioxo-1,2,3,6-tetrahydro-2,6-diaza-as-indacene-7-carbaldehyde;
8-Bromo-4-(2-chlorophenyl)-7-hydroxymethyl-6-[3-(2-methoxyethoxy)propyl]-6H-2,6-diaza-as-indacene-1,3-dione;
4-(2-Chlorophenyl)-6-methyl-1,3-dioxo-1,2,3,3a,4,5,6,8b-octahydro-2,6-diaza-as-indacene-7-carbaldehyde;
4-(2-Chlorophenyl)-6-methyl-1,3-dioxo-1,2,3,6-tetrahydro-2,6-diaza-as-indacene-7-carbaldehyde;
8-Bromo-4-(2-chlorophenyl)-6-methyl-1,3-dioxo-1,2,3,6-tetrahydro-2,6-diaza-as-indacene-7-carbaldehyde; or 8-Chloro-4-(2-chloro-phenyl)-6-methyl-1,3-dioxo-1,2,3,6-tetrahydro-2,6-diaza-as-indacene-7-carbaldehyde;
or a pharmaceutically acceptable salt, amide or ester form thereof.

11. A compound of claim 1 selected from the group of:
4-(2-Chlorophenyl)-7-hydroxymethyl-6-methyl-6H-2,6-diaza-as-indacene-1,3-dione;
8-Bromo-4-(2-chlorophenyl)-7-hydroxymethyl-6-methyl-6H-2,6-diaza-as-indacene-1,3-dione;
8-Chloro-4-(2-chlorophenyl)-7-hydroxymethyl-6-methyl-6H-2,6-diaza-as-indacene-1,3-dione;
6-[2-(tert-Butyldimethylsilanyloxy)ethyl]-4-(2-methoxyphenyl)-1,3-dioxo-1,2,3,3a,4,5,6,8b-octahydro-2,6-diaza-as-indacene-7-carbaldehyde;
6-[2-(tert-Butyldimethylsilanyloxy)ethyl]-4-(2-methoxyphenyl)-1,3-dioxo-1,2,3,6-tetrahydro-2,6-diaza-as-indacene-7-carbaldehyde;
8-Bromo-6-[2-(tert-butyldimethylsilanyloxy)ethyl]-4-(2-methoxyphenyl)-1,3-dioxo-1,2,3,6-tetrahydro-2,6-diaza-as-indacene-7-carbaldehyde;
8-Bromo-6-[2-(tert-butyldimethylsilanyloxy)ethyl]-7-hydroxymethyl-4-(2-methoxyphenyl)-6H-2,6-diaza-as-indacene-1,3-dione;
8-Bromo-6-(2-hydroxyethyl)-7-hydroxymethyl-4-(2-methoxyphenyl)-6H-2,6-diaza-as-indacene-1,3-dione;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid; or
(2-{[4-(2-Chlorophenyl)-6-methyl-1,3-dioxo-1,2,3,6-tetrahydro-2,6-diaza-as-indacene-7-carbonyl]amino}ethyl)carbamic acid;
or a pharmaceutically acceptable salt, amide or ester form thereof.

12. A compound of claim 1 selected from the group of:
4-(2-Chlorophenyl)-6-methyl-1,3-dioxo-1,2,3,6-tetrahydro-2,6-diaza-as-indacene-7-carboxylic acid;
4-(2-Chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Chloro-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid (2-hydroxyethyl)amide
4-(2-Chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
(3-{[4-(2-Chlorophenyl)-6-methyl-1,3-dioxo-1,2,3,6-tetrahydro-2,6-diaza-as-indacene-7-carbonyl]amino}propyl)carbamic acid;
(3-{[8-Bromo-4-(2-chlorophenyl)-6-methyl-1,3-dioxo-1,2,3,6-tetrahydro-2,6-diaza-as-indacene-7-carbonyl]amino}propyl)carbamic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydropyrrolo[3,4-e]indole-7-carboxylic acid;
8-Bromo-4-(2-chlorophenyl)-6-methyl-1,3-dioxo-1,2,3,6-tetrahydro-2,6-diaza-as-indacene-7-carboxylic acid; or
8-Bromo-4-(2-chlorophenyl)-6-methyl-1,3-dioxo-1,2,3,6-tetrahydro-2,6-diaza-as-indacene-7-carboxylic acid;
or a pharmaceutically acceptable salt, amide or ester form thereof.

13. A compound of claim 1 selected from the group of:
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid (2-aminoethyl)amide;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Bromo-4-(2-chlorophenyl)-6-methyl-1,3-dioxo-1,2,3,6-tetrahydro-2,6-diaza-as-indacene-7-carboxylic acid;
8-Chloro-4-(2-chlorophenyl)-6-methyl-1,3-dioxo-1,2,3,6-tetrahydro-2,6-diaza-as-indacene-7-carboxylic acid;
8-Chloro-4-(2-chlorophenyl)-6-methyl-1,3-dioxo-1,2,3,6-tetrahydro-2,6-diaza-as-indacene-7-carboxylic acid;
6-Methyl-1,3-dioxo-1,2,3,3a,4,5,6,8b-octahydro-2,6-diaza-as-indacene-4-carboxylic acid;
6-Methyl-1,3-dioxo-1,2,3,6-tetrahydro-2,6-diaza-as-indacene-4-carboxylic acid;
6-Methyl-1,3-dioxo-1,2,3,6-tetrahydro-2,6-diaza-as-indacene-4-carboxylic acid;
4-Iodo-6-methyl-6H-2,6-diaza-as-indacene-1,3-dione; 8-Bromo-4-iodo-6-methyl-6H-2,6-diaza-as-indacene-1,3-dione;
6-Methyl-4-(2,6-dimethoxyphenyl)-6H-2,6-diaza-as-indacene-1,3-dione;
6-Methyl-4-(2-methylsulfanylphenyl)-6H-2,6-diaza-as-indacene-1,3-dione;
4-(2-Methanesulfinylphenyl)-6-methyl-6H-2,6-diaza-as-indacene-1,3-dione; or
8-Bromo-4-(2-chlorophenyl)-6-[3-(cis-3,5-dimethylpiperazin-1-yl)propyl]-6H-2,6-diaza-as-indacene-1,3-dione;
or a pharmaceutically acceptable salt, amide or ester form thereof.

14. A compound of claim 1 selected from the group of:
6-(3-Aminopropyl)-8-bromo-4-(2-chlorophenyl)-6H-2,6-diaza-as-indacene-1,3-dione;
8-Bromo-4-(2-chlorophenyl)-6-(3-pyrrolidin-1-ylpropyl)-6H-2,6-diaza-as-indacene-1,3-dione;
8-Bromo-4-(2-chlorophenyl)-6-[3-(4-methylpiperazin-1-yl)-propyl]-6H-2,6-diaza-as-indacene-1,3-dione;
8-Bromo-4-(2-chlorophenyl)-6-(3-morpholin-4-ylpropyl)-6H-2,6-diaza-as-indacene-1,3-dione;
8-Bromo-4-(2-chlorophenyl)-6-(3-dimethylaminopropyl)-6H-2,6-diaza-as-indacene-1,3-dione;
8-Bromo-4-(2-chlorophenyl)-6-(3-dipropylaminopropyl)-6H-2,6-diaza-as-indacene-1,3-dione;
4-(2-Chlorophenyl)-6-(2-pyrrolidin-1-ylethyl)-6H-2,6-diaza-as-indacene-1,3-dione;
4-(2-Chlorophenyl)-6,8-dimethyl-1,3-dioxo-1,2,3,6-tetrahydro-2,6-diaza-as-indacene-7-carbaldehyde;
4-(2-Chlorophenyl)-7-hydroxymethyl-6,8-dimethyl-6H-2,6-diaza-as-indacene-1,3-dione;
4-(2-Chlorophenyl)-6-methyl-8-vinyl-6H-2,6-diaza-as-indacene-1,3-dione;
4-(2-Chlorophenyl)-8-(1,2-dihydroxyethyl)-6-methyl-6H-2,6-diaza-as-indacene-1,3-dione;
4-(2-Chlorophenyl)-8-(2-hydroxyethyl)-6-methyl-6H-2,6-diaza-as-indacene-1,3-dione;
4-(2-Chlorophenyl)-8-ethyl-6-methyl-6H-2,6-diaza-as-indacene-1,3-dione;
4-(2-Chlorophenyl)-8-ethynyl-6-methyl-6H-2,6-diaza-as-indacene-1,3-dione;
8-(3-Hydroxyprop-1-ynyl)-4-(2-methoxyphenyl)-6-methyl-6H-2,6-diaza-as-indacene-1,3-dione;
4-(2-Chlorophenyl)-6-methyl-1,3-dioxo-1,2,3,6-tetrahydro-2,6-diaza-as-indacene-8-carbonitrile;
4-(2-Chlorophenyl)-6-methyl-8-(3-oxobutyl)-6H-2,6-diaza-as-indacene-1,3-dione;
4-(2-Chlorophenyl)-8-(3-hydroxybutyl)-6-methyl-6H-2,6-diaza-as-indacene-1,3-dione;
4-(2-Chlorophenyl)-7-difluoromethyl-6-methyl-6H-2,6-diaza-as-indacene-1,3-dione; or
8-Chloro-4-(2-chlorophenyl)-7-difluoromethyl-6-methyl-6H-2,6-diaza-as-indacene-1,3-dione;
or a pharmaceutically acceptable salt, amide or ester form thereof.

15. A compound of claim 1 selected from the group of:
4-(2-Chlorophenyl)-7-(1-hydroxyethyl)-6-methyl-6H-2,6-diaza-as-indacene-1,3-dione;
8-Chloro-4-(2-chlorophenyl)-7-(1-hydroxyethyl)-6-methyl-6H-2,6-diaza-as-indacene-1,3-dione;
8-Bromo-4-(2-chlorophenyl)-6-methyl-1,3-dioxo-1,2,3,6-tetrahydro-2,6-diaza-as-indacene-7-carboxylic acid;
8-Chloro-4-(2-chlorophenyl)-6-methyl-1,3-dioxo-1,2,3,6-tetrahydro-2,6-diaza-as-indacene-7-carboxylic acid;
4-(2-Chlorophenyl)-6,8-dimethyl-1,3-dioxo-1,2,3,6-tetrahydro-2,6-diaza-as-indacene-7-carboxylic acid;
4-(2-Chlorophenyl)-7-(2-hydroxyethoxymethyl)-6-methyl-6H-2,6-diaza-as-indacene-1,3-dione;
4-(2-Chlorophenyl)-7-(2-methoxyethoxymethyl)-6-methyl-6H-2,6-diaza-as-indacene-1,3-dione;
8-Chloro-4-(2-chlorophenyl)-7-(2-hydroxyethoxymethyl)-6-methyl-6H-2,6-diaza-as-indacene-1,3-dione;
8-Bromo-4-(2-chlorophenyl)-7-(2-hydroxyethoxymethyl)-6-methyl-6H-2,6-diaza-as-indacene-1,3-dione;
3-[8-Bromo-4-(2-chlorophenyl)-1,3-dioxo-2,3-dihydro-1H-2,6-diaza-as-indacen-6-yl]propionic acid;
4-(2-Chlorophenyl)-6-methyl-8-pyrrolidin-1-ylmethyl-6H-2,6-diaza-as-indacene-1,3-dione;
4-(2-Chlorophenyl)-8-dimethylaminomethyl-6-methyl-6H-2,6-diaza-as-indacene-1,3-dione;
Acetic acid 4-(2-chlorophenyl)-6-methyl-1,3-dioxo-1,2,3,6-tetrahydro-2,6-diaza-as-indacen-8-ylmethyl ester; or
4-(2-chlorophenyl)-8-hydroxymethyl-6-methyl-6H-2,6-diaza-as-indacene-1,3-dione;
or a pharmaceutically acceptable salt, amide or ester form thereof.

16. A pharmaceutical composition comprising a pharmaceutically effective amount of a compound of claim 1, or a pharmaceutically acceptable salt form thereof, and a pharmaceutically acceptable carrier.

17. A method of treatment of proliferative disorders in a mammal, the method comprising administering to a mammal in need thereof a pharmaceutically effective amount of a compound of claim 1, or a pharmaceutically effective salt, ester or amide form thereof.

18. A method of inhibiting one or both of the checkpoint kinases Wee1 and Chk1 in a mammal, the method comprising administering to a mammal in need thereof a pharmaceutically effective amount of a compound of claim 1, or a pharmaceutically effective salt, ester or amide form thereof.
